SEQUENCE LISTING (25 sequences)

SEQ ID NO: 1 - DNA, 1674 bp, Mus musculus
  source 1..1674: mol_type = unassigned DNA
atgtacgccctcgccctcttcgccagccttctggccaccgctctgaccagccctgtccaagacccgaagacatgctctgggggctcagcagtgctgtgcagagatgtgaagacggcggtggactgtggggccgtgaagcactgccagcagatggtctggagcaagcccacagcgaaatcccttccttgcgacatatgcaaaactgttgtcaccgaagctgggaacttgctgaaagataatgctacgcaggaggagatccttcattacctggagaagacctgtgagtggattcatgactccagcctgtcggcctcgtgcaaggaggtggttgactcttacctgcctgtcatcctggacatgattaagggcgagatgagcaaccctggggaagtgtgctctgcgctcaacctctgccagtcccttcaggagtacttggccgagcaaaaccagaaacagcttgagtccaacaagatcccggaggtggacatggcccgtgtggttgcccccttcatgtccaacatccctctcctgctgtaccctcaggatcacccccgcagccagccccaacctaaggctaacgaggacgtctgccaggactgtatgaagctggtgtctgatgtccagactgctgtgaagaccaactccagctttatccagggcttcgtggaccacgtgaaggaggattgtgaccgcttggggccaggcgtgtctgacatatgcaagaactacgtggaccagtattccgaggtctgtgtccagatgttgatgcacatgcaggatcagcaacccaaggaaatctgtgtgctggctggcttctgtaatgaggtcaagagagtgccaatgaagactctggtccctgccaccgagaccattaagaacatcctccctgccctggagatgatggacccctatgagcagaatctggtccaggcccacaatgtgattttatgccagacctgtcagtttgtgatgaataagttttctgagctgattgtcaataatgccactgaggagctcctagttaaaggtttgagcaacgcatgcgcactgctccccgatcctgccagaaccaagtgccaggaggtggtgggaacatttggcccctccctgttggacatctttatccatgaggtaaaccccagctctctgtgcggtgtgatcggcctctgtgctgcccgcccggagttggtggaggcacttgagcagcctgcgccagccattgtatctgcactgctcaaagagcccacaccgccaaagcagcccgcacagcccaagcagtcggcattgcccgcccatgtgcctcctcagaagaatggtgggttctgtgaggtgtgcaagaaacctgtcctctatttggaacataacctggagaaaaacagcaccaaggaggaaatcctggccgcacttgagaagggctgcagcttcctgccagacccttaccagaagcagtgcgatgactttgtggctgagtatgagcccttgctattggagatcctcgtggaagtgatggatcctggatttgtgtgctcgaaaattggagtttgcccttctgcctataagctgctgctgggaaccgagaagtgtgtctggggccctagctactggtgtcagaacatggagactgccgcccgatgcaatgctgtcgatcattgcaaacgccatgtgtggaactag

SEQ ID NO: 2 - DNA, 1575 bp, Homo sapiens
  source 1..1575: mol_type = unassigned DNA
atgtacgccctcttcctcctggccagcctcctgggcgcggctctagccggcccggtccttggactgaaagaatgcaccaggggctcggcagtgtggtgccagaatgtgaagacggcgtccgactgcggggcagtgaagcactgcctgcagaccgtttggaacaagccaacagtgaaatcccttccctgcgacatatgcaaagacgttgtcaccgcagctggtgatatgctgaaggacaatgccactgaggaggagatccttgtttacttggagaagacctgtgactggcttccgaaaccgaacatgtctgcttcatgcaaggagatagtggactcctacctccctgtcatcctggacatcattaaaggagaaatgagccgtcctggggaggtgtgctctgctctcaacctctgcgagtctctccagaagcacctagcagagctgaatcaccagaagcagctggagtccaataagatcccagagctggacatgactgaggtggtggcccccttcatggccaacatccctctcctcctctaccctcaggacggcccccgcagcaagccccagccaaaggataatggggacgtttgccaggactgcattcagatggtgactgacatccagactgctgtacggaccaactccacctttgtccaggccttggtggaacatgtcaaggaggagtgtgaccgcctgggccctggcatggccgacatatgcaagaactatatcagccagtattctgaaattgctatccagatgatgatgcacatgcaacccaaggagatctgtgcgctggttgggttctgtgatgaggtgaaagagatgcccatgcagactctggtccccgccaaagtggcctccaagaatgtcatccctgccctggaactggtggagcccattaagaagcacgaggtcccagcaaagtctgatgtttactgtgaggtgtgtgaattcctggtgaaggaggtgaccaagctgattgacaacaacaagactgagaaagaaatactcgacgcttttgacaaaatgtgctcgaagctgccgaagtccctgtcggaagagtgccaggaggtggtggacacgtacggcagctccatcctgtccatcctgctggaggaggtcagccctgagctggtgtgcagcatgctgcacctctgctctggcacgcggctgcctgcactgaccgttcacgtgactcagccaaaggacggtggcttctgcgaagtgtgcaagaagctggtgggttatttggatcgcaacctggagaaaaacagcaccaagcaggagatcctggctgctcttgagaaaggctgcagcttcctgccagacccttaccagaagcagtgtgatcagtttgtggcagagtacgagcccgtgctgatcgagatcctggtggaggtgatggatccttccttcgtgtgcttgaaaattggagcctgcccctcggcccataagcccttgttgggaactgagaagtgtatatggggcccaagctactggtgccagaacacagagacagcagcccagtgcaatgctgtcgagcattgcaaacgccatgtgtggaactag

SEQ ID NO: 3 - protein, 557 aa, Mus musculus
MYALALFASLLATALTSPVQDPKTCSGGSAVLCRDVKTAVDCGAVKHCQQMVWSKPTAKSLPCDICKTVVTEAGNLLKDNATQEEILHYLEKTCEWIHDSSLSASCKEVVDSYLPVILDMIKGEMSNPGEVCSALNLCQSLQEYLAEQNQKQLESNKIPEVDMARVVAPFMSNIPLLLYPQDHPRSQPQPKANEDVCQDCMKLVSDVQTAVKTNSSFIQGFVDHVKEDCDRLGPGVSDICKNYVDQYSEVCVQMLMHMQDQQPKEICVLAGFCNEVKRVPMKTLVPATETIKNILPALEMMDPYEQNLVQAHNVILCQTCQFVMNKFSELIVNNATEELLVKGLSNACALLPDPARTKCQEVVGTFGPSLLDIFIHEVNPSSLCGVIGLCAARPELVEALEQPAPAIVSALLKEPTPPKQPAQPKQSALPAHVPPQKNGGFCEVCKKPVLYLEHNLEKNSTKEEILAALEKGCSFLPDPYQKQCDDFVAEYEPLLLEILVEVMDPGFVCSKIGVCPSAYKLLLGTEKCVWGPSYWCQNMETAARCNAVDHCKRHVWN

SEQ ID NO: 4 - protein, 527 aa, Homo sapiens [sequence omitted]

SEQ ID NO: 5 - protein, 527 aa, Homo sapiens
MYALFLLASLLGAALAGPVLGLKECTRGSAVWCQNVKTASDCGAVKHCLQTVWNKPTVKSLPCDICKDVVTAAGDMLKDNATEEEILVYLEKTCDWLPKPNMSASCKEIVDSYLPVILDIIKGEMSRPGEVCSALNLCESLQKHLAELNHQKQLESNKIPELDMTEVVAPFMANIPLLLYPQDGPRSKPQPKDNGDVCQDCIQMVTDIQTAVRTNSTFVQALVEHVKEECDRLGPGMADICKNYISQYSEIAIQMMMHMQDQQPKEICALVGFCDEVKEMPMQTLVPAKVASKNVIPALELVEPIKKHEVPAKSDVYCEVCEFLVKEVTKLIDNNKTEKEILDAFDKMCSKLPKSLSEECQEVVDTYGSSILSILLEEVSPELVCSMLHLCSGTRLPALTVHVTQPKDGGFCEVCKKLVGYLDRNLEKNSTKQEILAALEKGCSFLPDPYQKQCDQFVAEYEPVLIEILVEVMDPSFVCLKIGACPSAHKPLLGTEKCIWGPSYWCQNTETAAQCNAVEHCKRHVWN

SEQ ID NO: 6 - protein, 554 aa, Mus musculus
MYALALFASLLATALTSPVQDPKTCSGGSAVLCRDVKTAVDCGAVKHCQQMVWSKPTAKSLPCDICKTVVTEAGNLLKDNATQEEILHYLEKTCEWIHDSSLSASCKEVVDSYLPVILDMIKGEMSNPGEVCSALNLCQSLQEYLAEQNQKQLESNKIPEVDMARVVAPFMSNIPLLLYPQDHPRSQPQPKANEDVCQDCMKLVSDVQTAVKTNSSFIQGFVDHVKEDCDRLGPGVSDICKNYVDQYSEVCVQMLMHMQPKEICVLAGFCNEVKRVPMKTLVPATETIKNILPALEMMDPYEQNLVQAHNVILCQTCQFVMNKFSELIVNNATEELLVKGLSNACALLPDPARTKCQEVVGTFGPSLLDIFIHEVNPSSLCGVIGLCAARPELVEALEQPAPAIVSALLKEPTPPKQPAQPKQSALPAHVPPQKNGGFCEVCKKLVLYLEHNLEKNSTKEEILAALEKGCSFLPDPYQKQCDDFVAEYEPLLLEILVEVMDPGFVCSKIGVCPSAYKLLLGTEKCVWGPSYWCQNMETAARCNAVDHCKRHVWN

SEQ ID NO: 7 - protein, 557 aa, Rattus norvegicus
MYALALLASLLVTALTSPVQDPKICSGGSAVVCRDVKTAVDCRAVKHCQQMVWSKPTAKSLPCDICKTVVTEAGNLLKDNATEEEILHYLEKTCAWIHDSSLSASCKEVVDSYLPVILDMIKGEMSNPGEVCSALNLCQSLQEYLAEQNQRQLESNKIPEVDLARVVAPFMSNIPLLLYPQDRPRSQPQPKANEDVCQDCMKLVTDIQTAVRTNSSFVQGLVDHVKEDCDRLGPGVSDICKNYVDQYSEVAVQMMMHMQDQQPKEICVMVGFCDEVKRVPMRTLVPATEAIKNILPALELTDPYEQDVIQAQNVIFCQVCQLVMRKLSELIINNATEELLIKGLSKACSLLPAPASTKCQEVLVTFGPSLLDVLMHEVNPNFLCGVISLCSANPNLVGTLEQPAAAIVSALPKEPAPPKQPEEPKQSALRAHVPPQKNGGFCEVCKKLVIYLEHNLEKNSTKEEILAALEKGCSFLPDPYQKQCDEFVAEYEPLLLEILVEVMDPSFVCSKIGVCPSAYKLLLGTEKCVWGPGYWCQNMETAARCNAVDHCKRHVWN

SEQ ID NO: 8 - protein, 557 aa, Rattus rattus
MYALALLASLLVTALTSPVQDPKICSGGSAVVCRDVKTAVDCRAVKHCQQMVWSKPTAKSLPCDICKTVVTEAGNLLKDNATEEEILHYLEKTCAWIHDSSLSASCKEVVDSYLPVILDMIKGEMSNPGEVCSALNLCQSLQEYLAEQNQRQLESNKIPEVDLARVVAPFMSNIPLLLYPQDRPRSQPQPKANEDVCQDCMKLVTDIQTAVRTNSSFVQGLVDHVKEDCDRLGPGVSDICKNYVDQYSEVAVQMMMHMQDQQPKEICVMVGFCDEVKRVPMRTLVPATEAIKNILPALELTDPSEQDVIQAQNVIFCQVCQLVMRKLSELIINNATEELLIKGLSKACSLLPAPASTKCQEVLVTFGPSLLDVLMHEVNPNFLCGVISLCSASPNLVGTLEQPAAAIVSALPREPAPPKQPEEPKQSALRAHVPPQKNGGFCEVCKKLVIYLEHNLEKNSTKEEILAALEKGCSFLPDPYQKQCDDFVAEYEPLLLEILVEVMDPSFVCSKIGVCPSAYKLLLGTEKCVWGPGYWCQNMETAARCNAVDHCKRHVWN

SEQ ID NO: 9 - protein, 535 aa, Cavia porcellus
MFALFLLGSLLGTALASPVTDLRKCSGGSGVLCQDLKTAADCGAVQHCRQTVWSKPTVKSLPCDICKQVVTAAGDLLKDNGTEEEILSNLEKVCNWFSNPNLSSSCKEIVDSYLPVILDMIKGEASNPGEVCAALNLCESLQRHLAELNRQKQLEANKIPEADLSEVVAPFMANIPLLLYPQDGPQSQPQPKASGDVCQDCVQMVTDIQTAVRTNSTFVQGLVEHAKEECDRLGPGLADMCKNYISQYSEIAVQMMMHMQPKEICVLVGFCVEIKEVPMQPLVPARVATEVLPAVELVEPLEELVRAHESVTCKACEYVVKKVMELIDNNRTEEKIIHALDSVCALLPESVSEVCQEVVDTYGDSIVALLLQEMSPELVCSELGLCMSGKDRLSQPKLPMLRRLHAPPARVTPLKDGGFCEVCKKLVGYLEHNLEKNSTKQEILNALEKGCSLLPDPYQKQCDEFVSQYEPLLVEILVEVIDPSFVCTKIGACHSVRKLLLGTEKCVWGPSYWCHNMETAAQCNAVEHCKRHVWN

SEQ ID NO: 10 - protein, 528 aa, Oryctolagus cuniculus
MYALFFLAGLLGTGLASPVLGLKECTRGSAVWCQNVKTAAECGAVKHCLQTVWSKPTVKSLPCRLCKDVISAVGEMLKDNATEEEILVYLERTCDWLPNSNLSASCKEIVDSYLPVILDLIKGEASRPGEVCSALSLCQSLQKHLAELSQRKQLASNKIPELDMPEVVAPFMANIPLLLYPQDGPRSKPQPKDDGDVCQDCVQMVTDIQNAVRTNSTFVETLVEHAKEQCDRLGPGVADMCKNYINQYSEIAVQMMMHMQPKEICGLVGFCDQVKEMPMQALVPAKAAAENVVPALELVEPVKERPVQARSDLLCKACELVVKKVVDLIDNNKTEEEIIHGLDKVCSELPASISQECQEVVDTYGSAILSVLRQEVDPELVCHIFHLCSSGSTPPRLPVVPARVAPVKDGGFCEVCKKLVSYLEHNLEKNSTKQEILAALEKGCSFLPDPYQKQCDQFVTEYEPVLIEVLVEVMDPAFVCLKIGACPSGPKPLLGTEKCVWGPSYWCQNSETAAQCNAVEHCRRHVWN

SEQ ID NO: 11 - protein, 527 aa, Macaca mulatta
MYALFLLASLLGAALASPVLGMKECTRGSAVWCQNVKTASDCGAVKHCLQTVWNKPTVKSLPCDICKDVVTAAGDMLKDNATEEEILVYLEKTCDWLPKPNMSASCKEIVDSYLPVILDIIKGEMSRPGEVCSALNLCESLQKHLAELNHQKQLESNKIPELDMTEVVAPFMANIPLLLYPQDGPRSKPQPKDNGDVCQDCIQMVTDIQTAVRTNSTFVQALVEHVKEECDRLGPGMADMCKNYISQYSEIAIQMMMHMQDQQPKEICALVGFCDEVKEMPMQTLVPAKVASKNVIPALELVEPIKKHEVPAKSDVYCEVCEFLVKEVTKLIDNNKTEKEILDTFDKMCSKLPKSLSEECQEVVDTYGSSILSILLQEVSPELVCSMLRLCSGTRLPALTVHVTQPKDGGFCEVCKKLVGYLDRNLEKNSTKQEILAALEKGCSFLPDPYQKQCDQFVAEYEPVLIEILVEVMDPSFVCLKIGACPSAHKPLLGTEKCVWGPSYWCQNTETAAQCNAVEHCKRHVWN

SEQ ID NO: 12 - protein, 34 aa, unidentified
GPKRCTWGPSVWCQNLETASQCNAVKHCLQRVWS

SEQ ID NO: 13 - protein, 33 aa, unidentified
TKECTWGPSYWCQSLETAKECGAVEHCQQHVWN

SEQ ID NO: 14 - protein, 75 aa, unidentified
LLCELCEFVVKQLENLLKDNKTEEEIKKALEKVCKKLPKSLSDQCKEFVDQYGPEIIDLLEQGLDPKDVCQKLGL

SEQ ID NO: 15 - RNA, 6932 nt, Homo sapiens
  source 1..6932: mol_type = mRNA
aggccgcgctcagcaggcggggcgggagccgcgtgcgcccgaggacccggccggaaggcttgcgccagctcaggatgaggacaggctgggcgacccctcgccgcccggcggggctcctcatgctgctcttctggttcttcgatctcgcggagccctctggccgcgcagctaatgaccccttcaccatcgtccatggaaatacgggcaagtgcatcaagccagtgtatggctggatagtagcagacgactgtgatgaaactgaggacaagttatggaagtgggtgtcccagcatcggctctttcatttgcactcccaaaagtgccttggcctcgatattaccaaatcggtaaatgagctgagaatgttcagctgtgactccagtgccatgctgtggtggaaatgtgagcaccactctctgtacggagctgcccggtaccggctggctctgaaggatggacatggcacagcaatctcaaatgcatctgatgtctggaagaaaggaggctcagaggaaagcctttgtgaccagccttatcatgagatctataccagagatgggaactcttatgggagaccttgtgaatttccattcttaattgatgggacctggcatcatgattgcattcttgatgaagatcatagtgggccatggtgtgccaccaccttaaattatgaatatgaccgaaagtggggcatctgcttaaagcctgaaaacggttgtgaagataattgggaaaagaacgagcagtttggaagttgctaccaatttaatactcagacggctctttcttggaaagaagcttatgtttcatgtcagaatcaaggagctgatttactgagcatcaacagtgctgctgaattaacttaccttaaagaaaaagaaggcattgctaagattttctggattggtttaaatcagctatactctgctagaggctgggaatggtcagaccacaaaccattaaactttctcaactgggatccagacaggcccagtgcacctactataggtggctccagctgtgcaagaatggatgctgagtctggtctgtggcagagcttttcctgtgaagctcaactgccctatgtctgcaggaaaccattaaataatacagtggagttaacagatgtctggacatactcagatacccgctgtgatgcaggctggctgccaaataatggattttgctatctgctggtaaatgaaagtaattcctgggataaggcacatgcgaaatgcaaagccttcagtagtgacctaatcagcattcattctctagcagatgtggaggtggttgtcacaaaactccataatgaggatatcaaagaagaagtgtggataggccttaagaacataaacataccaactttatttcagtggtcagatggtactgaagttactctaacatattgggatgagaatgagccaaatgttccctacaataagacgcccaactgtgtttcctacttaggagagctaggtcagtggaaagtccaatcatgtgaggagaaactaaaatatgtatgcaagagaaagggagaaaaactgaatgacgcaagttctgataagatgtgtcctccagatgagggctggaagagacatggagaaacctgttacaagatttatgaggatgaggtcccttttggaacaaactgcaatctgactatcactagcagatttgagcaagaatacctaaatgatttgatgaaaaagtatgataaatctctaagaaaatacttctggactggcctgagagatgtagattcttgtggagagtataactgggcaactgttggtggaagaaggcgggctgtaaccttttccaactggaattttcttgagccagcttccccgggcggctgcgtggctatgtctactggaaagtctgttggaaagtgggaggtgaaggactgcagaagcttcaaagcactttcaatttgcaagaaaatgagtggaccccttgggcctgaagaagcatcccctaagcctgatgacccctgtcctgaaggctggcagagtttccccgcaagtctttcttgttataaggtattccatgcagaaagaattgtaagaaagaggaactgggaagaagctgaacgattctgccaagcccttggagcacacctttctagcttcagccatgtggatgaaataaaggaatttcttcactttttaacggaccagttcagtggccagcattggctgtggattggtttgaataaaaggagcccagatttacaaggatcctggcaatggagtgatcgtacaccagtgtctactattatcatgccaaatgagtttcagcaggattatgacatcagagactgtgctgctgtcaaggtatttcataggccatggcgaagaggctggcatttctatgatgatagagaatttatttatttgaggccttttgcttgtgatacaaaacttgaatgggtgtgccaaattccaaaaggccgtactccaaaaacaccagactggtacaatccagaccgtgctggaattcatggacctccacttataattgaaggaagtgaatattggtttgttgctgatcttcacctaaactatgaagaagccgtcctgtactgtgccagcaatcacagctttcttgcaactataacatcttttgtgggactaaaagccatcaaaaacaaaatagcaaatatatctggtgatggacagaagtggtggataagaattagcgagtggccaatagatgatcattttacatactcacgatatccatggcaccgctttcctgtgacatttggagaggaatgcttgtacatgtctgccaagacttggcttatcgacttaggtaaaccaacagactgtagtaccaagttgcccttcatctgtgaaaaatataatgtttcttcgttagagaaatacagcccagattctgcagctaaagtgcaatgttctgagcaatggattccttttcagaataagtgttttctaaagatcaaacccgtgtctctcacattttctcaagcaagcgatacctgtcactcctatggtggcacccttccttcagtgttgagccagattgaacaagactttattacatccttgcttccggatatggaagctactttatggattggtttgcgctggactgcctatgaaaagataaacaaatggacagataacagagagctgacgtacagtaactttcacccattattggttagtgggaggctgagaataccagaaaatttttttgaggaagagtctcgctaccactgtgccctaatactcaacctccaaaaatcaccgtttactgggacgtggaattttacatcctgcagtgaacgccactttgtgtctctctgtcagaaatattcagaagttaaaagcagacagacgttgcagaatgcttcagaaactgtaaagtatctaaataatctgtacaaaataatcccaaagactctgacttggcacagtgctaaaagggagtgtctgaaaagtaacatgcagctggtgagcatcacggacccttaccagcaggcattcctcagtgtgcaggcgctccttcacaactcttccttatggatcggactcttcagtcaagatgatgaactcaactttggttggtcagatgggaaacgtcttcattttagtcgctgggctgaaactaatgggcaactcgaagactgtgtagtattagacactgatggattctggaaaacagttgattgcaatgacaatcaaccaggtgctatttgctactattcaggaaatgagactgaaaaagaggtcaaaccagttgacagtgttaaatgtccatctcctgttctaaatactccgtggataccatttcagaactgttgctacaatttcataataacaaagaataggcatatggcaacaacacaggatgaagttcatactaaatgccagaaactgaatccaaaatcacatattctgagtattcgagatgaaaaggagaataactttgttcttgagcaactgctgtacttcaattatatggcttcatgggtcatgttaggaataacttatagaaataagtctcttatgtggtttgataagaccccactgtcatatacacattggagagcaggaagaccaactataaaaaatgagaagtttttggctggtttaagtactgacggcttctgggatattcaaacctttaaagttattgaagaagcagtttattttcaccagcacagcattcttgcttgtaaaattgaaatggttgactacaaagaagaatataatactacactgccacagtttatgccatatgaagatggtatttacagtgttattcaaaaaaaggtaacatggtatgaagcattaaacatgtgttctcaaagtggaggtcacttggcaagcgttcacaaccaaaatggccagctctttctggaagatattgtaaaacgtgatggatttccactatgggttgggctctcaagtcatgatggaagtgaatcaagttttgaatggtctgatggtagtacatttgactatatcccatggaaaggccaaacatctcctggaaattgtgttctcttggatccaaaaggaacttggaaacatgaaaaatgcaactctgttaaggatggtgctatttgttataaacctacaaaatctaaaaagctgtcccgtcttacatattcatcaagatgtccagcagcaaaagagaatgggtcacggtggatccagtacaagggtcactgttacaagtctgatcaggcattgcacagtttttcagaggccaaaaaattgtgttcaaaacatgatcactctgcaactatcgtttccataaaagatgaagatgagaataaatttgtgagcagactgatgagggaaaataataacattaccatgagagtttggcttggattatctcaacattctgttgaccagtcttggagttggttagatggatcagaagtgacatttgtcaaatgggaaaataaaagtaagagtggtgttggaagatgtagcatgttgatagcttcaaatgaaacttggaaaaaagttgaatgtgaacatggttttggaagagttgtctgcaaagtgcctctgggccctgattacacagcaatagctatcatagttgccacactaagtatcttagttctcatgggcggactgatttggttcctcttccaaaggcaccgtttgcacctggcgggtttctcatcagttcgatatgcacaaggagtgaatgaagatgagattatgcttccttctttccatgactaaattcttctaaaagttttctaatttgcactaatgtgttatgagaaattagtcacttaaaatgtcccagtgtcagtatttactctgctccaaagtagaactcttaaatactttttcagttgtttagatcttaggcatgtgctggtatccacagttaattccctgctaaatgccatgtttatcaccctaattaatagaatggaggggactccaaagctggaactgaagtccaaattgtttgtacagtaatatgtttaatgttcattttctctgtatgaatgtgattggtaactaggatatgtatattttaatagaatttttaacaaaacttcttagaaaattaaaataggcatattactaggtgacatgtctactttttaatttttaagagcatccggccaaatgcaaaattagtacctcaaagtaaaaattgaactgtaaactctatcagcattgtttcaaaatagtcatttttagcactggggaaaaataaacaataagacatgcttactttttaatttttatttttttgagactgagtctctctctgttgcccaggctggagtacaatggcgtgatctcggctcactgcaaatctccgcctcccaggttcaagcgattctcctgcctcagcctcctgagtagctgggattacaggcaactgccaccatgcccggctaatttttgtatttttagtagagatggggtttcaccatgttggccaggctggtctcgaactcgtgaccgcaggtgatcctcccgcctcggcctcccaaagtgctgggattacaggcatgagccaccgcgcctggcctctgcttactttttatatagcaaaatgattcctcttggcaagatgtttcttatattattccaaagttatttcataccattattatgtaaatatgaagagtttttttctgtttataattgtttataaaacaatgacttttaaagatttagtgcttaacattttcccaagtgtgggaacattatttttagattgagtaggtaccttgtagcagtgtgctttgcattttctgatgtattacatgactgtttcttttgtaaagagaatcaactaggtatttaagactgataattttacaatttatatgcttcacatagcatgtcaacttttgactaagaattttgttttacttttttaacatgtgttaaacagagaaagggtccatgaaggaaagtgtatgagttgcatttgtaaaaatgagactttttcagtggaactctaaaccttgtgatgactactaacaaatgtaaaattatgagtgattaagaaaacattgctttgtggttatcactttaagttttgacacctagattatagtcttagtaatagcatccactggaaaaggtgaaaatgttttattcggcatttaacttacatttgtactttatttttgtataaaatccatagatttattttacatttagagtatttacactatgataaagttgtaaataattttctaagacagtttttatatagtctacagttgtcctgatttcttattgaatttgttagactagttctcttgtcctgtgatctgtgtacaattttagtcactaagactttcctccaagaactaagccaacttgatgtgaaaagcacagctgtatataatggtgatgtcataataaagttgttttatcttttaagtaaaagtaaaa

SEQ ID NO: 16 - RNA, 6871 nt, Mus musculus
  source 1..6871: mol_type = mRNA
accgctgaagccggtgcgggcaggagaggcttgggactggagatgcggacgggccgggtgaccccgggcctggcggcggggctactcctgctgttgctgcggtccttcgggcttgtggagccttctgagagctcaggtaatgatccattcaccatcgtccatgaaaacactggcaagtgcatccagccgctgtctgactgggtagtggcccaggactgtagcggaactaacaacatgttgtggaagtgggtgtcccagcaccgcctctttcacctggaatcccagaagtgcctcggcctcgatattaccaaagccacggacaacctgcgaatgttcagctgtgactccaccgtcatgctgtggtggaaatgtgagcaccattcgctgtacaccgctgcccagtacaggctagctctgaaagatggatatgccgtagccaatacgaatacatctgatgtctggaagaagggaggctccgaggaaaacctttgtgcccagccttatcatgagatatacaccagagatgggaattcctacgggagaccttgtgaattccctttcttgattggtgagacatggtaccatgactgcattcatgatgaagatcatagtgggccatggtgtgccactaccctaagttatgaatatgatcaaaagtggggcatctgcctactaccagaaagtggctgtgaaggtaactgggaaaagaatgagcagattggaagttgctaccaatttaataatcaggaaattctgtcttggaaagaagcttatgtttcctgtcagaaccaaggagctgacttactgagcatccacagtgctgccgaattagcctacattacgggaaaagaggacattgctagacttgtttggcttggactgaatcagctctattctgcgagaggttgggaatggtcagacttcaggccactcaaatttcttaactgggatccaggcacgcccgttgcacctgtgattggtgggtcaagctgtgccagaatggacacagagtccgggctgtggcaaagtgtttcctgtgaatctcagcagccttacgtctgcaagaagccactgaacaacacgctggagctcccagatgtttggacttacacagatacccactgccatgtgggctggctgccaaataatgggttttgctatctgctggcgaatgaaagtagttcctgggatgcagcacatttgaaatgcaaagccttcggtgcagacctcatcagcatgcactccttagcagatgtggaggtggttgtcacgaaactccataatggggatgtcaaaaaagaaatatggacaggccttaaaaacacaaacagccctgctttgttccagtggtcggacggaacggaagttactctaacgtactggaatgagaatgagccgagtgttcccttcaacaagactcccaactgtgtttcctatttaggaaagttaggtcagtggaaagtccagtcctgtgagaagaaactcagatatgtatgcaagaaaaagggagaaataactaaggatgcagagtcggataagctgtgtccgccagacgagggctggaagagacatggagaaacctgttacaagatttatgagaaagaggcccctttcggaacgaactgcaacctgaccatcactagcaggttcgagcaggaattcttgaattatatgatgaagaactatgataagtcccttcggaagtacttctggactggcctgagagaccctgactctcgaggagaatacagttgggccgttgctcagggagtaaagcaggctgtgaccttttccaactggaattttcttgaaccggcgtctccaggcgggtgcgtggctatgtctactggaaagactcttggcaagtgggaagtgaagaactgcagaagcttccgtgctctttcaatatgcaagaaagtgagcgaaccccaggagcctgaagaagcagcccccaagcccgacgacccctgtcctgaaggctggcacactttcccctccagcctttcttgttataaggtgttccatatagaaagaatcgtaagaaagaggaactgggaagaagccgaaaggttctgccaagcccttggagctcacctacccagcttcagtcgtagagaggaaattaaggactttgtgcatttgttaaaggaccagttcagtgggcagcgttggttgtggattggtctgaataagagaagccctgatttacaagggtcctggcagtggagtgaccggacaccagtgtctgctgtgatgatggagccggagtttcaacaggattttgacatcagagactgtgctgccatcaaggtccttgatgtaccttggcgaagagtctggcatctctatgaggacaaggactatgcttactggaaaccttttgcttgtgatgccaagcttgagtgggtgtgccagattccaaaaggtagcactccccagatgccagactggtataatccagagcgcactggaattcatgggcccccagttataattgaaggaagtgaatactggtttgttgctgatccccacttaaactacgaagaagccgtcttatactgtgctagcaatcacagctttcttgccacgataacatcgttcacaggactaaaagctatcaaaaacaaactagcaaatatttctggcgaggaacagaagtggtgggtgaaaacgagtgagaatccaattgatcgttactttctaggctcgcgccgccgcctgtggcaccatttccccatgacgtttggagatgaatgtttgcacatgtcagccaagacgtggcttgttgacttaagtaaacgagcggactgtaatgccaagttgcccttcatctgtgaaagatacaatgtctcttcattagagaaatacagcccagatcctgcagccaaagtacagtgcactgagaagtggattccttttcaaaataagtgcttcctaaaggtcaactctgggcccgttacgttttctcaagcaagcggcatttgtcattcctacggcggcacccttccttccgtgctgagccggggtgaacaagatttcattatatccttgcttcctgaaatggaagctagtctatggattggtctgcgctggactgcctacgaaaggataaacagatggacagacaacagagagctgacctacagcaactttcacccactgctggtcggtcggaggctgagcataccaacgaatttctttgatgatgagtcccacttccactgcgccttgattcttaatctcaaaaagtcaccgcttactgggacctggaattttacttcctgttcagaacgacactctctgtctctctgtcaaaaatactcagagactgaagacggacagccctgggagaacacttcaaaaacagtgaagtatctaaataacctatacaaaatcatctcgaagcccctgacgtggcacggcgctctgaaggagtgcatgaaagagaagatgaggttggtgagcatcacagacccttaccagcaggccttcctcgcagtgcaggccaccctgcgcaacagctccttctggatcggactctccagtcaagatgatgaactcaactttggttggtcagatgggaaacgtcttcaatttagtaactgggctggaagcaatgagcaacttgatgactgcgtgatattagacacagatggattctggaaaacagctgactgtgatgataaccagcctggcgccatttgctactatccaggaaatgagactgaggaggaggtcagagcactggacactgctaaatgcccgtctcctgtacagagcaccccatggataccattccagaactcctgctacaatttcatgattaccaacaacaggcataagacagtcacaccggaggaagtgcagtccacgtgcgagaagctgcattcgaaagcacacagtctgagcattcggaatgaggaggagaatacctttgttgtggaacagcttctgtacttcaattatattgcctcatgggtcatgttaggaataacctatgaaaacaattctttgatgtggtttgataaaactgcattgtcctacacacactggagaacgggaagaccaactgtgaaaaatggcaaatttttggctggtctaagtactgatggattctgggatattcagtctttcaatgttattgaagaaacacttcatttttaccagcacagtatttctgcttgtaaaattgaaatggttgactatgaggacaaacacaatggcaccctgccacagttcattccatataaggacggcgtctacagcgttattcagaagaaggtgacgtggtatgaagcattgaacgcgtgctctcaaagtgggggagagttggccagtgttcacaacccaaatgggaagctctttctggaagacattgtgaaccgtgacggattccctctctgggttgggctctcaagtcatgatggaagcgaatcgagtttcgaatggtccgatggcagagcatttgactatgtcccatggcagagcctacaatctcccggagactgtgtcgtcttatatccaaaaggaatttggagacgtgaaaaatgcctgtctgttaaggatggtgctatttgttacaagcctacaaaagataaaaagctgatctttcatgtaaaatcatcaaaatgtccagtggcaaagagggatggtccccagtgggtccagtatgggggccactgttacgcttcggaccaggtactgcacagcttctcagaggccaaacaagtgtgtcaagagcttgatcattcggcaactgttgtcaccatagcagatgaaaatgagaataagtttgtgagcagactgatgagggagaactataatattactatgagagtttggcttggcctgtctcagcattcactcgatcagtcttggagttggctcgatggattagatgtgacatttgtcaaatgggaaaataaaactaaggatggtgatgggaaatgtagcattttaatagcttcaaatgaaacctggagaaaagtccattgctcacgtggctatgcaagagctgtctgcaaaattcctctgagcccggactacacaggcatagccatcctgtttgccgtgctgtgcctcttagggctcatcagcttggcgatttggttcctcttgcaacgatcccatatccgctggaccggcttctcctcggttcggtatgaacatggaaccaacgaagacgaggtgatgctcccttctttccacgactaacttcttccaagagtttgttcattggccctggatgttgcggaagcaagatgagccacttagcactgcatccccccaatcagtgtctaactttgtttctaagtagagaagtcttagagatgtattcagcgggcgggaggggagagttgctcagctgatgccctaacggccatgctaatgagtcagttatcagagcagagagacttaaagccaaaatggatattcaggttgttcacagggggataaatttagctaatgtgaattttctctgtaaaaatgcagttgctaaatagatacgtacatattgtaatagcattgtaacagcctcatagaaaattaaaactgtcagtacaagtaggctccacatttaccttttatgggcatctgaccaaatgcaaagttagtaactccagcaaaaactcaactataaataagatcaatgatgcttcaaaatagccttttaaggcactggggaaaaaatacaaaataaggcatattttttatacagaaaacagagccccttggaagatgtttcttatactatttcaaagatatgtatactggtattaattaattgttacatttctgtttttcattgcttatattcagtgagttggtttttgtctttaacttaaagttaatgtttcctaaagttgggaacgttgctgttaagttgcctgggcgtgctgtatcagcatgtctgcgtttgatgattttcttttttacaaggacccaattacagctgagacttcagcttttacagttcctgtctttcacataggagatcagctttctgttggtggtggctggggtttttggttttgttttgttgtttttgtttttgttttgttttgttttttgaaacatggtttctttgtgcatcatagccctggctgtcctggactgactttgtagaccaggctagccactaattcagagatcccccgcctctgccttgggagtgctgggatcaaagactgtgctggcaccaccccagcaagcaggcagggtatcaccttttgatgaatgtattatgttgttgcctttttacttgagcgaaacataaaagtgatctttgaataatgttgtttgctacttctgtatttgtaaacaatcaccgtccctaagacaatgaaactcagaactctgtgattcctgtggaaatatttaaaactgaaatgattagtgacacactattttgttgatgtaatttccatttgaacatctggaatatacttttactagtagtgtcaaaataaatatggtgactgttttattcagtgtttcatatttatactttagagtatttttgtacaagatccagatttattttgcttgaggtgtttatgctatcataaagttgtaaataattttctaagactgttttaatgcaatctacagttttcctaaaatatttttcttgaggcctcttttaactatcactaggagtttcttcaaaaaactaaatcaataccatgtgaccacagttgtgatcatggctgcttccgtggctgatccttgcatgtgaaagcatctctaaattaggtttgattgtatcccatgtgtagtatgaagagaaggtgtccagctattgcgagttaaggtattttaagactttgtgagcactttttaaagaataaatctttgtatgtctaaatta

SEQ ID NO: 17 - protein, 1722 aa, Homo sapiens
MRTGWATPRRPAGLLMLLFWFFDLAEPSGRAANDPFTIVHGNTGKCIKPVYGWIVADDCDETEDKLWKWVSQHRLFHLHSQKCLGLDITKSVNELRMFSCDSSAMLWWKCEHHSLYGAARYRLALKDGHGTAISNASDVWKKGGSEESLCDQPYHEIYTRDGNSYGRPCEFPFLIDGTWHHDCILDEDHSGPWCATTLNYEYDRKWGICLKPENGCEDNWEKNEQFGSCYQFNTQTALSWKEAYVSCQNQGADLLSINSAAELTYLKEKEGIAKIFWIGLNQLYSARGWEWSDHKPLNFLNWDPDRPSAPTIGGSSCARMDAESGLWQSFSCEAQLPYVCRKPLNNTVELTDVWTYSDTRCDAGWLPNNGFCYLLVNESNSWDKAHAKCKAFSSDLISIHSLADVEVVVTKLHNEDIKEEVWIGLKNINIPTLFQWSDGTEVTLTYWDENEPNVPYNKTPNCVSYLGELGQWKVQSCEEKLKYVCKRKGEKLNDASSDKMCPPDEGWKRHGETCYKIYEDEVPFGTNCNLTITSRFEQEYLNDLMKKYDKSLRKYFWTGLRDVDSCGEYNWATVGGRRRAVTFSNWNFLEPASPGGCVAMSTGKSVGKWEVKDCRSFKALSICKKMSGPLGPEEASPKPDDPCPEGWQSFPASLSCYKVFHAERIVRKRNWEEAERFCQALGAHLSSFSHVDEIKEFLHFLTDQFSGQHWLWIGLNKRSPDLQGSWQWSDRTPVSTIIMPNEFQQDYDIRDCAAVKVFHRPWRRGWHFYDDREFIYLRPFACDTKLEWVCQIPKGRTPKTPDWYNPDRAGIHGPPLIIEGSEYWFVADLHLNYEEAVLYCASNHSFLATITSFVGLKAIKNKIANISGDGQKWWIRISEWPIDDHFTYSRYPWHRFPVTFGEECLYMSAKTWLIDLGKPTDCSTKLPFICEKYNVSSLEKYSPDSAAKVQCSEQWIPFQNKCFLKIKPVSLTFSQASDTCHSYGGTLPSVLSQIEQDFITSLLPDMEATLWIGLRWTAYEKINKWTDNRELTYSNFHPLLVSGRLRIPENFFEEESRYHCALILNLQKSPFTGTWNFTSCSERHFVSLCQKYSEVKSRQTLQNASETVKYLNNLYKIIPKTLTWHSAKRECLKSNMQLVSITDPYQQAFLSVQALLHNSSLWIGLFSQDDELNFGWSDGKRLHFSRWAETNGQLEDCVVLDTDGFWKTVDCNDNQPGAICYYSGNETEKEVKPVDSVKCPSPVLNTPWIPFQNCCYNFIITKNRHMATTQDEVHTKCQKLNPKSHILSIRDEKENNFVLEQLLYFNYMASWVMLGITYRNKSLMWFDKTPLSYTHWRAGRPTIKNEKFLAGLSTDGFWDIQTFKVIEEAVYFHQHSILACKIEMVDYKEEYNTTLPQFMPYEDGIYSVIQKKVTWYEALNMCSQSGGHLASVHNQNGQLFLEDIVKRDGFPLWVGLSSHDGSESSFEWSDGSTFDYIPWKGQTSPGNCVLLDPKGTWKHEKCNSVKDGAICYKPTKSKKLSRLTYSSRCPAAKENGSRWIQYKGHCYKSDQALHSFSEAKKLCSKHDHSATIVSIKDEDENKFVSRLMRENNNITMRVWLGLSQHSVDQSWSWLDGSEVTFVKWENKSKSGVGRCSMLIASNETWKKVECEHGFGRVVCKVPLGPDYTAIAIIVATLSILVLMGGLIWFLFQRHRLHLAGFSSVRYAQGVNEDEIMLPSFHD

SEQ ID NO: 18 - protein, 1723 aa, Mus musculus
MRTGRVTPGLAAGLLLLLLRSFGLVEPSESSGNDPFTIVHENTGKCIQPLSDWVVAQDCSGTNNMLWKWVSQHRLFHLESQKCLGLDITKATDNLRMFSCDSTVMLWWKCEHHSLYTAAQYRLALKDGYAVANTNTSDVWKKGGSEENLCAQPYHEIYTRDGNSYGRPCEFPFLIGETWYHDCIHDEDHSGPWCATTLSYEYDQKWGICLLPESGCEGNWEKNEQIGSCYQFNNQEILSWKEAYVSCQNQGADLLSIHSAAELAYITGKEDIARLVWLGLNQLYSARGWEWSDFRPLKFLNWDPGTPVAPVIGGSSCARMDTESGLWQSVSCESQQPYVCKKPLNNTLELPDVWTYTDTHCHVGWLPNNGFCYLLANESSSWDAAHLKCKAFGADLISMHSLADVEVVVTKLHNGDVKKEIWTGLKNTNSPALFQWSDGTEVTLTYWNENEPSVPFNKTPNCVSYLGKLGQWKVQSCEKKLRYVCKKKGEITKDAESDKLCPPDEGWKRHGETCYKIYEKEAPFGTNCNLTITSRFEQEFLNYMMKNYDKSLRKYFWTGLRDPDSRGEYSWAVAQGVKQAVTFSNWNFLEPASPGGCVAMSTGKTLGKWEVKNCRSFRALSICKKVSEPQEPEEAAPKPDDPCPEGWHTFPSSLSCYKVFHIERIVRKRNWEEAERFCQALGAHLPSFSRREEIKDFVHLLKDQFSGQRWLWIGLNKRSPDLQGSWQWSDRTPVSAVMMEPEFQQDFDIRDCAAIKVLDVPWRRVWHLYEDKDYAYWKPFACDAKLEWVCQIPKGSTPQMPDWYNPERTGIHGPPVIIEGSEYWFVADPHLNYEEAVLYCASNHSFLATITSFTGLKAIKNKLANISGEEQKWWVKTSENPIDRYFLGSRRRLWHHFPMTFGDECLHMSAKTWLVDLSKRADCNAKLPFICERYNVSSLEKYSPDPAAKVQCTEKWIPFQNKCFLKVNSGPVTFSQASGICHSYGGTLPSVLSRGEQDFIISLLPEMEASLWIGLRWTAYERINRWTDNRELTYSNFHPLLVGRRLSIPTNFFDDESHFHCALILNLKKSPLTGTWNFTSCSERHSLSLCQKYSETEDGQPWENTSKTVKYLNNLYKIISKPLTWHGALKECMKEKMRLVSITDPYQQAFLAVQATLRNSSFWIGLSSQDDELNFGWSDGKRLQFSNWAGSNEQLDDCVILDTDGFWKTADCDDNQPGAICYYPGNETEEEVRALDTAKCPSPVQSTPWIPFQNSCYNFMITNNRHKTVTPEEVQSTCEKLHSKAHSLSIRNEEENTFVVEQLLYFNYIASWVMLGITYENNSLMWFDKTALSYTHWRTGRPTVKNGKFLAGLSTDGFWDIQSFNVIEETLHFYQHSISACKIEMVDYEDKHNGTLPQFIPYKDGVYSVIQKKVTWYEALNACSQSGGELASVHNPNGKLFLEDIVNRDGFPLWVGLSSHDGSESSFEWSDGRAFDYVPWQSLQSPGDCVVLYPKGIWRREKCLSVKDGAICYKPTKDKKLIFHVKSSKCPVAKRDGPQWVQYGGHCYASDQVLHSFSEAKQVCQELDHSATVVTIADENENKFVSRLMRENYNITMRVWLGLSQHSLDQSWSWLDGLDVTFVKWENKTKDGDGKCSILIASNETWRKVHCSRGYARAVCKIPLSPDYTGIAILFAVLCLLGLISLAIWFLLQRSHIRWTGFSSVRYEHGTNEDEVMLPSFHD

SEQ ID NO: 19 - protein, 448 aa, synthetic construct
EVKLLESGGGLVQPGGSLRLSCAASGFTFNDFYMNWIRQPPGQAPEWLGVIRNKGNGYTTEVNTSVKGRFTISRDNTQNILYLQMNSLRAEDTAIYYCARGGPYYYSGDDAPYWGQGVMVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPPVSSVFIFPPKPKDVLTITLTPKVTCVVVDISKDDPEVQFSWFVDDVEVHTAQTQPREEQFNSTFRSVSELPIMHQDWLNGKAFACAVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 20 - protein, 214 aa, synthetic construct
DIQMTQFPSFLSTSLGNSITITCHASQNIKGWLAWYQQKSGNAPQLLIYKAFSLQSGVPSRFSGSGSGTDYIFTISNLQPEDIATYYCQHYQSFPWTFGGGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 21 - protein, 10 aa, synthetic construct
ELAGIGILTV

SEQ ID NO: 22 - protein, 9 aa, synthetic construct
SLLMWITQV

SEQ ID NO: 23 - protein, 9 aa, synthetic construct
YMDGTMSQV

SEQ ID NO: 24 - protein, 9 aa, synthetic construct
ITDQVPFSV

SEQ ID NO: 25 - protein, 8 aa, synthetic construct
SIINFEKL

(19) United States
(12) Patent Application Publication
(10) Pub. No.: US 2026/0069705 A1
(43) Pub. Date: Mar. 12, 2026

(54) METHODS AND COMPOSITIONS FOR ENHANCED ANTIGEN PRESENTATION IN THE TUMOR MICROENVIRONMENT

(71) Applicant: THE CHILDREN'S MEDICAL CENTER CORPORATION, Boston, MA (US)

(72) Inventors: Florian WINAU, Boston, MA (US); Pankaj SHARMA, Boston, MA (US)

(73) Assignee: THE CHILDREN'S MEDICAL CENTER CORPORATION, Boston, MA (US)

(21) Appl. No.: 18/872,644

(22) PCT Filed: Jun. 9, 2023
(86) PCT No.: PCT/US2023/068165

§ 371 Date: Dec. 6, 2024

Related U.S. Application Data
(60) Provisional application No. 63/350,734, filed on Jun. 9, 2022.

(51) Int. Cl. A61K 47/68; A61P 35/00; C07K 16/28

(52) U.S. Cl. CPC A61K 47/6811; A61K 47/6849; A61P 35/00; C07K 16/2851

(57) ABSTRACT

Provided herein are compositions and methods for the delivery of prosaposin and saposins within the tumor microenvironment in order to enhance anti-tumor immunity.

### CROSS REFERENCE TO RELATED APPLICATIONS

This application claims benefit under 35 U.S.C. § 119(e) of U.S. Provisional Application No. 63/350,734, filed Jun. 9, 2022, the contents of which are incorporated herein by reference in its entirety.

### SEQUENCE LISTING

The instant application contains a Sequence Listing that has been submitted in XML format via Patent Center and is hereby incorporated by reference in its entirety. Said XML copy, created on Jun. 1, 2023, is named “701039-192200WOPT_SL.xml” and is 48,645 bytes in size.

### TECHNICAL FIELD

The technology described herein relates to improving immunotherapy of cancer.

### BACKGROUND

The harnessing of immunological mechanisms for cancer therapy is revolutionizing oncology treatment. Immune checkpoint blockade has shown that effective CD8 T cell responses are vital to protect against tumors. Central to robust T cell activation is their interaction with antigen-presenting cells, such as dendritic cells (DCs), which trigger efficient T cell priming and effector functions required for cancer protection. Unfortunately, antigen presentation and T cell activation in the tumor microenvironment (TME) are often suppressed, a phenomenon that facilitates immune escape of cancer and precludes effective therapy and cure.

### SUMMARY

The methods and compositions provided herein are based, in part, on the discovery that saposins within the tumor microenvironment enhance antigen presentation of cancer antigens, thereby enhancing anti-tumor immunity. Accordingly, provided herein are methods and compositions for increasing expression or levels of saposins in the tumor microenvironment.

In one aspect as described herein is a composition comprising: a saposin or prosaposin targeted for delivery to a dendritic cell.

In one embodiment of any of the aspects described herein, the saposin or prosaposin comprises an antigen binding moiety that binds a dendritic cell antigen.

In another embodiment of any of the aspects described herein, the saposin or prosaposin is chemically conjugated to the antigen binding moiety, optionally via a chemical or peptide linker.

In another embodiment of any of the aspects described herein, the dendritic cell antigen comprises DEC205.

In another embodiment of any of the aspects described herein, the antigen binding moiety comprises an antibody or antigen binding fragment thereof.

In another embodiment of any of the aspects described herein, the saposin comprises saposin A, saposin B, saposin C, and/or saposin D.

In another embodiment of any of the aspects described herein, the composition further comprises a pharmaceutically acceptable carrier.

In another aspect described herein is a fusion protein comprising a saposin or prosaposin conjugated to a moiety that targets the fusion protein to dendritic cells.

In another aspect described herein is a method of treating cancer comprising administering the composition of any one of the embodiments to a subject in need thereof.

In another aspect described herein is a method of enhancing anti-tumor immunity in a subject, the method comprising administering a composition of any one of the embodiments described herein to a subject having cancer, wherein the anti-tumor immunity is increased by at least 10% as compared to the anti-tumor immunity in a subject not administered the composition of any one of the embodiments described herein.

In one embodiment of any of the aspects described herein, the anti-tumor immunity is assessed by measuring T lymphocyte stimulation.

In another embodiment of any of the aspects described herein, the tumor volume is decreased in the subject by at least 20%.

In another embodiment of any of the aspects described herein, the cancer is melanoma.

In another embodiment of any of the aspects described herein, antigen presentation by dendritic cells is increased.

In another embodiment of any of the aspects described herein, the saposin or prosaposin degrades apoptotic vesicles from the tumor.

### Definitions

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, a “subject” means a human or animal. Usually the animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of cellular replacement therapy. A subject can be male or female.

A subject can be one who has been previously diagnosed with or identified as suffering from or having a condition in need of treatment (e.g. cancer, etc.) or one or more complications related to such a condition, and optionally, have already undergone treatment for cancer or the one or more complications related to cancer. Alternatively, a subject can also be one who has not been previously diagnosed as having a cancer or one or more complications related to a cancer. For example, a subject can be one who exhibits one or more risk factors for a cancer or one or more complications related to a cancer or a subject who does not exhibit risk factors.

A “subject in need” of treatment for a particular condition (e.g., cancer) can be a subject having that condition, diagnosed as having that condition, or at risk of developing that condition.

A variant amino acid or DNA sequence can be at least 85%, at least 87%, at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are very well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties. Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. cancer. The term “treating” includes reducing or alleviating at least one adverse effect or symptom of a condition, disease or disorder associated with a cancer. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “administering,” refers to the placement of a therapeutic agent as disclosed herein into a subject by a method or route which results in at least partial delivery of the agent at a desired site. Pharmaceutical compositions comprising the compounds disclosed herein can be administered by any appropriate route which results in an effective treatment in the subject. In some embodiments, administration comprises physical human activity, e.g., an injection, act of ingestion, an act of application, and/or manipulation of a delivery device or machine. Such activity can be performed, e.g., by a medical professional and/or the subject being treated.

As used herein, “contacting” refers to any suitable means for delivering, or exposing, an agent to at least one cell. Exemplary delivery methods include, but are not limited to, direct delivery to cell culture medium, perfusion, injection, or other delivery method well known to one skilled in the art. In some embodiments, contacting comprises physical human activity, e.g., an injection; an act of dispensing, mixing, and/or decanting; and/or manipulation of a delivery device or machine.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

As used herein, the term “corresponding to” refers to an amino acid or nucleotide at the enumerated position in a first polypeptide or nucleic acid, or an amino acid or nucleotide that is equivalent to an enumerated amino acid or nucleotide in a second polypeptide or nucleic acid. Equivalent enumerated amino acids or nucleotides can be determined by alignment of candidate sequences using degree of homology programs known in the art, e.g., BLAST.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

In some embodiments, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

### BRIEF DESCRIPTION OF THE DRAWINGS

FIG. 1 shows a sequence alignment of prosaposin in Mus musculus, Rattus norvegicus, Rattus rattus, Cavia porcellus, Oryctolagus cuniculus, Homo sapiens, and Macaca mulatta.

FIG. 2 examines the conserved domains of prosaposin, which comprise SapA domains, and saposin A, saposin B, saposin C, and saposin D, which comprise SapB1 and SapB2 domains.

FIG. 3A-3G show saposins promote cross-presentation of membrane-associated tumor antigen. (FIG. 3A) Diagram depicting the experimental read-outs used in FIG. 3A-3G. MCA101 fibrosarcoma cells were γ-irradiated (100 Gy), prior to collection of apoptotic vesicles from supernatant and analysis using electron microscopy (EM) and calcein leakage assay. Moreover, apoptotic MCA101 cells expressing membrane-associated ovalbumin were used to pulse bone marrow-derived DCs from WT or pSAP-KO mice, prior to analysis of digestion of apoptotic cells using confocal microscopy, and antigen processing and T cell activation using FACS. (FIG. 3B) Calcein leakage assay to quantify the effect of saposins on disintegration of apoptotic bodies. Apoptotic vesicles were prepared using differential ultracentrifugation (100,000 g) from supernatant of irradiated MCA101 cells and visualized using transmission electron microscopy (left image). Scale bar=200 nm. Apoptotic bodies were further loaded with calcein dye prior to incubation with indicated saposins or BSA (negative control), and calcein release was quantified in the supernatant using fluorimetry. Depicted is % leakage compared to 100% lysis induced by Triton X-100. (FIG. 3C) Representative confocal microscopy image showing colocalization of apoptotic bodies with LAMP-1. WT DCs were pulsed with CFSE-labeled, γ-irradiated apoptotic MCA101 tumor cells for 2 hours. ApoBD=apoptotic body. (FIG. 3D) Representative confocal microscopy images showing the kinetics of apoptotic cell disintegration in WT or pSAP-KO DCs. DCs were pulsed with CFSE-labeled, γ-irradiated apoptotic MCA101 tumor cells for 2 hours, and the numbers of apoptotic bodies (ApoBD) were quantified at indicated time points using ImageJ software. (FIG. 3E) Representative histogram overlays and bar graph showing flow cytometry staining and mean fluorescence intensity (MFI) of MHC-I-SIINFEKL peptide on the surface of WT or pSAP-KO DCs after incubation with either soluble OVA (sOVA) or irradiated MCA101-OVA tumor cells (mOVA=membrane-associated OVA) for 4 hours. (FIG. 3F) Histograms and bar graph showing frequencies of proliferating, CFSE^{low} CD8 T cells after 3-day coculture with WT or pSAP-KO DCs pulsed with soluble OVA. (FIG. 3G) Histograms and bar graph depicting the frequencies of CFSE^{low} CD8 T cells after 3-day coculture with WT or pSAP-KO DCs pulsed with irradiated MCA101-OVA cells. In addition, pSAP-KO DCs were reconstituted with 10 μg/ml of recombinant prosaposin prior to the T cell assay. Data shown in all graphs represent mean±SD from 3-5 independent replicates. p-values were determined using unpaired Student's t-test. *p<0.05; **p<0.01; ***p<0.001; ns: not significant.

FIG. 4A-4H demonstrates prosaposin is required for tumor immunity and boosts T cells from melanoma patients. (FIG. 4A) Experimental scheme of tumor challenge studies. WT and pSAP-KO BM chimeric mice were primed with 4×10⁵ γ-irradiated MCA101-OVA cells (s.c.) and subsequently inoculated with 1×10⁶ live MCA101-OVA cells (s.c.) 7 days post priming. (FIG. 4B) Comparison of tumor sizes between WT and pSAP-KO mice on day 17 (left) and kinetics of tumor growth (right). (FIG. 4C) Representative histogram overlay and bar graph depicting the staining and mean fluorescence intensity (MFI) of MHC-I-SIINFEKL peptide on the surface of tumor DCs from pSAP-KO or WT animals. (FIG. 4D) FACS plots and bar graphs showing frequencies of MHC-I (Kb-SIINFEKL) tetramer- and IFN-γ-positive tumor-infiltrating CD8 T cells in pSAP-KO or WT mice. MHC-I tetramer specifically detects CD8 T cells reactive with SIINFEKL peptide. (FIG. 4E) Experimental set-up for the coculture of myeloid and CD8 T cells isolated from human melanoma. Single cell suspensions from human melanoma samples were FACS-sorted for CD146⁺ melanoma cells, CD8⁺ T cells, and CD11c/b⁺ myeloid cells. CD146⁺ cells were γ-irradiated and incubated with DCs, which were further cocultured with CD8 T cells in the presence or absence of recombinant pSAP. (FIG. 4F) FACS plots and bar graph showing the frequencies of IFN-γ-positive CD8 T cells following the indicated culture conditions. (FIG. 4G) Representative histogram overlay and bar graph demonstrating surface staining and MFI of LAMP-1 on CD8 T cells according to the indicated culture conditions. (FIG. 4H) Flow cytometry analysis and summarizing bar graph depicting the frequencies of antigen-specific CD8 T cells reactive with HLA-A*0201 tetramers loaded with epitopes from gp100, MART-1, Tyrosinase, and NY-ESO-1, following the indicated culture set-ups. Data shown in FIG. 4B-4D are representative of three independent experiments, while 4F-4H depict mean±SD from 7 independent subjects. p-values were determined by unpaired Student's t-test in graphs B-D, while one-way ANOVA was used in graphs F-H. **p<0.01; ***p<0.001; ****p<0.0001.

FIG. 5A-5K examines hyperglycosylation of prosaposin in tumor DCs leads to its secretion. WT mice were inoculated with 1×10⁶ live MCA101 cells, and 18 days post tumor inoculation, cDC1 and cDC2 populations were FACS-sorted from tumor and spleen. (FIG. 5A) Immunoblot showing the expression of pSAP and saposins in tumor and splenic DC subsets. pSAP-75=hyperglycosylated prosaposin, pSAP-65=glycosylated pSAP, and SAPs=saposins. (FIG. 5B) Quantification of prosaposin secreted by DCs. FACS-sorted splenic and tumor DC subsets were cultured in cRPMI for 48 hours, and pSAP in culture supernatant was quantified using ELISA. (FIG. 5C) Immunoblot of Endo H-treated pSAP. Left: Mechanism of Endo H that leads to cleavage of high-mannose but not complex glycans. Right: FACS-sorted DCs from tumor and spleen were lysed in RIPA buffer, and cell lysates were treated with Endo H for 12 hours at 37° C., prior to analysis using immunoblot. (FIG. 5D) MALDI-TOF mass spectrometry analysis of permethylated N-linked glycans of prosaposin immunoprecipitated from FACS-sorted CD11c⁺ DCs. Enzymatically released N-glycans from pSAP of splenic (top panel) and tumor (bottom panel) DCs were analyzed. Glycan compositions were assigned based on m/z values. x-axis: mass to charge ratio (m/z). y-axis: signal intensity of the ions. (FIG. 5E) Heat map of differentially expressed genes in tumor DCs involved in glycosylation, as analyzed by real-time RT² profiler PCR array. Splenic DCs were used as control to calculate fold change in gene expression. (FIG. 5F). Bar graph depicting glycosyltransferase and glycosidase gene expression in tumor compared to splenic DC1. (FIG. 5G) Proximity ligation assay (PLA) of pSAP and sortilin. Confocal microscopy images of tumor and splenic DC subsets reveal PLA signal between pSAP and sortilin. The violin plot shows quantification of PLA signal, where 200 cells from each sample were analyzed for statistics. (FIG. 5H) Co-immunoprecipitation of sortilin and pSAP in tumor and splenic DCs. Top panel shows blot of sortilin pulled down by anti-pSAP antibody. Bottom panel demonstrates immunoblot of total sortilin in corresponding DC populations. (FIG. 5I) PLA of pSAP and sortilin in human melanoma and monocyte-derived DCs. Melanoma DCs were sorted as CD11c⁺ cells from viable CD45⁺ cells isolated from human melanoma samples, while monocyte-derived DCs (MoDCs) were generated by culturing monocytes with interleukin 4 (IL-4) and granulocyte-macrophage colony-stimulating factor (GM-CSF) for 4 days. The violin plot shows quantification of PLA signal, where 200 cells from each sample were analyzed for statistics. (FIG. 5J) Immunoblot of pSAP in human melanoma DCs and MoDCs. pSAP-75=hyperglycosylated prosaposin; pSAP-65=glycosylated pSAP. (FIG. 5K) Illustration visualizing glycosylation mechanisms that control prosaposin trafficking in tumor DCs. Hyperglycosylation of prosaposin compromises its interaction with sortilin and reroutes it to the secretory pathway. Data shown in all graphs are representative of three independent experiments, and p-values were determined using unpaired Student's t-test. *p<0.05; **p<0.01; ***p<0.001; ****p<0.0001.

FIG. 6A-6I shows TGF-β induces prosaposin hyperglycosylation in DCs and compromises tumor immunity. (FIG. 6A) Immunoblot showing dose-dependent induction of pSAP hyperglycosylation and secretion by the murine DC line (DC2.4) incubated with recombinant TGF-β for 48 hours. pSAP-75=hyperglycosylated prosaposin, pSAP-65=glycosylated pSAP. (FIG. 6B) Quantification of pSAP in the culture supernatant of DC2.4 cells after incubation with recombinant TGF-β for 2 days as measured using ELISA. (FIG. 6C) Scatter plot showing the correlation of gene expression of glycosyltransferases and glycosidases between tumor DCs and TGF-β-stimulated DC2.4 cells. mRNA fold changes were quantified by real-time RT² profiler PCR array. CD11c tumor DCs were analyzed using splenic DCs as control, while TGF-β-stimulated DC2.4 cells were compared with sham-treated DC2.4 cells. (FIG. 6D) Experimental scheme of tumor cell challenge. WT (Tgfbr2^{f/f}) and CD11c-Cre×Tgfbr2^{f/f} (Tgfbr2^{Δ}^{^{DC}}) BM chimeric mice were inoculated with 1×10⁶ live MCA101-OVA cells (s.c.). (FIG. 6E) Kinetics of tumor growth in control Cre⁻ and CD11c-Cre×Tgfbr2^{f/f} mice. (FIG. 6F) Histogram overlay and bar graph depicting H-2K^{b}-SIINFEKL staining and mean fluorescence intensity (MFI) on tumor DCs from Tgfbr2^{Δ}^{^{DC}} mice or Tgfbr2^{f/f} controls on day 20 after tumor cell injection. (FIG. 6G) FACS plots and bar graph showing frequencies of IFN-γ⁺ tumor-infiltrating CD8 T cells in Tgfbr2^{Δ}^{^{DC}} or Tgfbr2^{f/f} animals on day 20 post tumor challenge. (FIG. 6H) Immunoblot of pSAP and saposins in tumor DCs from Tgfbr2^{Δ}^{^{DC}} or Tgfbr2^{f/f} mice on day 20 after tumor cell inoculation. pSAP-75=hyperglycosylated prosaposin, pSAP-65=glycosylated pSAP, and SAPs=saposins. (FIG. 6I) Differentially expressed glycosyltransferase and glycosidase genes in tumor DCs isolated from Tgfbr2^{Δ}^{^{DC}} or Tgfbr2^{f/f} mice on day 20 after tumor challenge. Splenic DCs from Tgfbr2^{f/f} animals were used as control to calculate mRNA fold change. Data shown in all graphs are representative of three independent experiments, and p-values were determined using unpaired Student's t-test. *p<0.05; **p<0.01; ***p<0.001; ****p<0.0001.

FIG. 7A-7H examines the reconstitution of tumor DCs with recombinant prosaposin drives protection from cancer. (FIG. 7A) Regime of pSAP targeting to tumor DCs. pSAP KO BM-chimeric mice were inoculated with 1×10⁶ live MCA101-OVA cells. On day 9 and 13 after tumor cell injection, mice were intravenously treated with pSAP coupled with either anti-DEC205 or isotype control antibodies. (FIG. 7B) FACS plots and bar graph showing the amount of prosaposin uptake by DCs at the tumor site as analyzed on day 14 after tumor challenge. (FIG. 7C) Experimental set-up depicting tumor cell inoculation and pSAP targeting via DEC205 in WT mice. (FIG. 7D) Comparison of tumor sizes on day 20 (left) and kinetics of tumor growth (right). (FIG. 7E) Histogram overlay and bar graph showing H-2K^{b}-SIINFEKL peptide surface staining and MFI on tumor DCs on day 20 post tumor challenge. (FIG. 7F) FACS plots and bar graphs showing percentages of IFN-γ-positive CD8 T cells in tumors and tumor-draining lymph nodes (dLN) in mice treated with pSAP coupled to anti-DEC205 or isotype control. (FIG. 7G) Flow cytometry and bar graphs showing MHC-I (K^{b}-SIINFEKL) tetramer-positive CD8 T cells in tumors and draining lymph nodes (dLN). (FIG. 7H) Experimental set-up depicting B16F10 melanoma cell inoculation, pSAP targeting via DEC205, and tumor growth kinetics. WT mice were inoculated with 3×10⁶ live melanoma cells and were treated with pSAP coupled with either anti-DEC205 or isotype control antibodies, either alone or in combination with anti-PD-L1 antibodies. Statistical analysis of tumor volume across all treatment groups is shown on day 30 after tumor challenge. Data shown in all graphs are representative of three independent experiments, and p-values were determined using unpaired Student's t-test. **P<0.01; ***P<0.001; ****P<0.0001.

FIGS. 8A-8C shows prosaposin is required for cross-priming of CD8 T cells. (FIG. 8A) Experimental set-up. WT and pSAP-KO mice were intravenously injected with CFSE-labeled OT-I T cells, followed by subcutaneous administration of 5×10⁶ irradiated MCA101-OVA cells on day 1 after adoptive T cell transfer. iLN=inguinal lymph node. (FIG. 8B) Histogram overlay and bar graph showing H-2K^{b}-SIINFEKL peptide staining and mean fluorescence intensity (MFI) on migratory DCs. Migratory DCs were gated as CD 11c and MHC-IIhi^{gh} cells. (FIG. 8C) FACS plots and bar graphs showing frequencies of CFSE^{low} CD8 T cells (top panel) and IFN-γ-positive CD8 T cells (bottom panel) in iLN on day 5 after T cell transfer. Data shown are representative of 7 independent biological replicates, and p-values were determined using unpaired Student's t-test. ***p<0.001.

FIGS. 9A-9E demonstrates cancer growth and immunity are controlled by prosaposin. (FIG. 9A) Experimental scheme of tumor cell injections. WT and pSAP-KO BM chimeric mice were inoculated with 1×10⁶ live MCA101-OVA cells (s.c.), and mice were sacrificed 17 days later for tumor harvest and cellular analysis. (FIG. 9B) Comparison of tumor sizes on day 17 after challenge (left) and kinetics of tumor growth (right). (FIG. 9C) Histogram overlay and bar graph depicting H-2K^{b}-SIINFEKL peptide staining and mean fluorescence intensity (MFI) on tumor DCs. (FIG. 9D) FACS plots and bar graphs showing frequencies of MHC-I (K^{b}-SIINFEKL) tetramer- and IFN-γ-positive tumor-infiltrating CD8 T cells. (FIG. 9E) Histogram overlay and bar graph depicting LAMP-1 staining and MFI on the surface of tumor-infiltrating CD8 T cells. Data shown are representative of three independent experiments. Statistical analysis was performed using unpaired Student's t-test in graphs C-E. **p<0.01; ***p<0.001.

FIG. 10 analyzes FACS sorting of tumor, myeloid, and T cells from human melanoma patients. FACS plots show the hierarchical gating strategy to identify melanoma (CD146⁺), myeloid (CD45⁺, CDIIc/b⁺), and cytotoxic T cells (CD45⁺, CD8⁺), sub-gated from viable cells after doublet discrimination using FSC-A vs. FSC-H gating. Three-way sorting was used to separate the indicated populations, and purity was assessed using post-sort analysis based on flow cytometry.

FIG. 11A-11E shows Tumor DCs are impaired in antigen processing and presentation. (FIG. 11A) Experimental set-up and flow cytometry gating strategy to identify and isolate cDC1 and cDC2 populations from tumor, draining lymph nodes (dLN), and spleen. WT mice were inoculated with 1×10⁶ live MCA101-OVA cells (s.c.), and tumor, dLN, and spleen were isolated on day 17 after tumor challenge. ResDCs=resident DCs, MigDCs=migratory DCs. (FIG. 11B) Antigen uptake assay using FITC-dextran. FACS-sorted DC subsets were incubated with FITC-dextran for 45 minutes, and background signal was quenched by trypan blue staining. Mean fluorescence intensity (MFI) of FITC was quantified using flow cytometry. (FIG. 11C) Antigen processing assay using DQ-OVA. FACS-sorted DC subsets were incubated with DQ-OVA for 1 hour, and MFI of DQ-OVA was quantified using flow cytometry. DQ-OVA is a self-quenching reagent which fluoresces after proteolytic cleavage in lysosomes. (FIG. 11D) Antigen presentation assay using soluble OVA or irradiated tumor cells. Purified DCs were pulsed either with soluble OVA (sOVA, left panel), or with γ-irradiated MCA101-OVA cells (mOVA, right panel) for 4 hours. Antigen presentation was quantified using H-2K^{b}-SIINFEKL staining on DCs and is depicted in histograms and bar graphs plotting MFI. (FIG. 11E) T cell assay using OT-I T cells stimulated by cDC1 and cDC2 purified from spleen or the tumor microenvironment (TME). DCs were pulsed with γ-irradiated MCA101-OVA cells for 4 hours and were subsequently cocultured with CFSE-labeled OT-I T cells for 3 days. CFSE dilutions were measured by flow cytometry to assess CD8 T cell proliferation. Data in all graphs show mean±SD, and p-values were calculated using one-way ANOVA. ***p<0.001.

FIG. 12 examines TGF-β treatment changes expression profile of glycosylation genes in DC2.4 cells. Heat map of differentially expressed genes involved in glycosylation in DC2.4 cells after TGF-β treatment. DC2.4 cells were treated with 10 ng/ml TGF-β for 48 hours, and mRNA fold change was determined using the real-time RT² profiler PCR array compared with untreated DC2.4 cells.

FIGS. 13A-13F shows targeting of pSAP to DCs via anti-DEC205 promotes antigen cross-presentation. (FIG. 13A) Native electrophoresis and immunoblot of pSAP coupled to isotype or anti-DEC205. Antibody-coupled pSAP was detected at molecular weight >180 kD (lane A and B), while pSAP was detected as 65 kD band (lane C). (FIG. 13B) ELISA-based quantification of the amount of prosaposin coupled to anti-DEC205 or isotype control antibody. ELISA plates were coated with anti-IgG capture antibodies, followed by incubation with isotype- or anti-DEC205-coupled pSAP. Anti-pSAP antibody was used for detection. (FIG. 13C) FACS plots and bar graph showing engagement of anti-DEC205/pSAP conjugates on the DC surface. In a primary step, BMDCs were incubated for 15 minutes using either isotype- or anti-DEC205-coupled pSAP, or unconjugated DEC205 antibody that served as positive control. In a subsequent step, fluorochrome-tagged secondary antibodies were used to detect pSAP/antibody conjugates or DEC205 on the surface of DCs. (FIG. 13D) Experimental set-up showing pulsing of pSAP-KO BMDCs with γ-irradiated MCA101-OVA cells, followed by coculture with OT-I T cells in the presence of either isotype- or anti-DEC205-coupled pSAP. (FIG. 13E) Histogram overlay and bar graph depicting MHC-I-restricted antigen presentation (H-2K^{b}-SIINFEKL staining) by pSAP-KO BMDCs pulsed with γ-irradiated MCA101-OVA cells and incubated with either isotype- or anti-DEC205-coupled pSAP using indicated concentrations. Analysis by flow cytometry and quantification using mean fluorescence intensity (MFI). (FIG. 13F) FACS histogram overlay and bar graph showing CD69 staining and MFI on CD8 T cells cocultured with pSAP-KO BMDCs treated with pSAP/antibody conjugates at indicated concentrations. Data in all graphs depict mean±SD, and p-values were determined using unpaired Student's t-test. ***p<0.001; ****p<0.0001.

FIG. 14 shows the mechanism of prosaposin-based immune escape and its targeting for immunotherapy of cancer. Left side: Hyperglycosylation of prosaposin and immune evasion. TGF-β induces hyperglycosylation of prosaposin in tumor DCs, leading to its secretion. As a consequence, reduced availability of saposins in the endolysosomal compartment impairs antigen processing and presentation, causing hampered T cell responses and loss of tumor control. Right side: Therapeutic targeting of tumor DCs with recombinant prosaposin. To overcome immune escape in cancer, recombinant prosaposin can be targeted to endolysosomes in tumor DCs via the endocytic receptor DEC205. Delivery of functional prosaposin restores antigen processing and presentation, triggering reinvigorated T cell responses to promote tumor immunity.

FIG. 15 examines the disintegration of lipid bilayers by saposins A-D (SAP-A, SAP-B, SAP-C, and SAP-D).

FIG. 16 shows efficient CD8 T cell activation mediated by pSAP, SAP-C, and SAP-D.

FIG. 17 examines Anti-tumor activity of DC-targeted prosaposin and SAP-D.

FIG. 18 shows endocytic receptors expressed by DCs.

### DETAILED DESCRIPTION

Provided herein are methods and compositions for enhancing anti-tumor immunity in a subject by administering a saposin or prosaposin that is modified to specifically target dendritic cells.

Saposins or prosaposins can be targeted to dendritic cells, for example, by conjugating or otherwise attaching a saposin or prosaposin to a moiety that specifically binds a cell-surface marker on the dendritic cell. As used herein, a “cell-surface marker” refers to any molecule that is expressed on the surface of a cell (e.g., a dendritic cell). Cell-surface expression usually requires that a molecule possesses a transmembrane domain. Many naturally occurring cell-surface markers are termed “CD” or “cluster of differentiation” molecules. Cell-surface markers often provide antigenic determinants to which antibodies can bind.

In the methods and compositions provided herein, cell surface markers of dendritic cells are contemplated for use in promoting delivery of saposin or prosaposin to dendritic cells. Exemplary dendritic cell markers include, but are not limited to, ADAM19/MADDAM, BDCA-2, CD1a, CD11c, CD21, CD83, CD86, CLIP17-/restin, clusterin, DC-LAMP/CD208, DEC205, estrogen receptor-alpha, fascin, HLA-DR, NLDC-145, CLEC9A and S-100. It should be considered whether binding of a targeting moiety to a dendritic cell surface marker will interfere with the function of the dendritic cell. In this regard, however, cell-surface proteins that participate in delivering protein antigens to the endocytic system are of particular interest—that is, where the normal function of the cell-surface protein is to deliver antigens to the endocytic system for processing, it can actually be beneficial to target saposins or prosaposin to the dendritic cell using a binding moiety that recognizes such a cell-surface marker. A cell-surface marker of particular relevance to the methods described herein is CD205 (also referred to herein as DEC205).

### Prosaposins/Saposins

Prosaposin is a highly conserved glycoprotein precursor that can be cleaved to four different products, saposins A, B, C, and D. Saposin is an acronym for Sphingolipid Activator PrO[S]teINs. Each domain of the precursor protein is approximately 80 amino acid residues long with nearly identical placement of cysteine residues and glycosylation sites. Typically, saposins A-D localize to the lysosomal compartment where they facilitate the catabolism of glycosphingolipids with short oligosaccharide groups. However, in the tumor microenvironment the secretion of saposins is increased as a result of hyperglycosylation of prosaposin, thus reducing the level of saposins in the lysosomes, which can result in uncontrolled tumor growth. Increasing expression of saposins in the lysosome is shown herein to degrade apoptotic bodies in the tumor microenvironment, thereby exposing cancer antigens for presentation by immune cells and in turn increasing anti-tumor immunity.

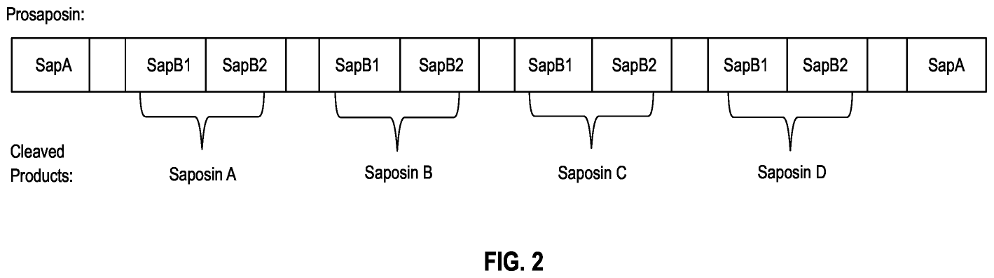

A saposin (Saposins A-D), a prosaposin, or a related protein (SAPLIP) contains evolutionary-conserved protein domains known as a saposin-domain. There are 3 different kinds of saposin domains: Saposin A domain (SapA), Saposin B1 domain (SapB1), and Saposin B2 (SapB2). All mammalian saposins are synthesized in vivo as a single precursor molecule (prosaposin) which contains four Saposin-B domains, yielding the active saposins after proteolytic cleavage, and two Saposin-A domains that are removed in the activation reaction. The mammalian prosaposin includes the N- and C-terminal SapA domains, both of which are proteolytically cleaved as the proprotein matures. Four connected pairs of SapB1-SapB2 domains are released, sequentially named Saposin-A through D. A schematic of this is shown in FIG. 2. The structure of prosaposin and saposins A-D are better understood by analyzing the SapA, SapB1, and SapB2 domains to understand what residues are conserved and what residues can be manipulated or varied and still maintain function. Additional references that discuss the saposin domains include Munford R S, Sheppard P O, O'Hara P J. “Saposin-like proteins (SAPLIP) carry out diverse functions on a common backbone structure.” J Lipid Res. 1995 August; 36(8):1653-63. PMID: 7595087 and Staab J F, Ginkel D L, Rosenberg G B, Munford R S. “A saposin-like domain influences the intracellular localization, stability, and catalytic activity of human acyloxyacyl hydrolase.” J Biol Chem. 1994 Sep. 23; 269(38):23736-42. PMID: 8089145, which are incorporated by reference herein in their entireties.

Modifications to prosaposin to remove glycosylation sites or to introduce lysosomal signaling peptides are specifically contemplated herein. In some embodiments, prosaposin can be codon optimized. In some embodiments, the prosaposin is human prosaposin. It is contemplated that the prosaposin, or saposins derived therefrom, can be non-human, e.g., derived from other mammalian sources to the extent that the non-human prosaposin or saposin retains the same function as the human in promoting tumor antigen presentation by dendritic cells. Human prosaposin coding sequence is provided herein as SEQ ID NO: 2, and human prosaposin protein sequence is provided herein as SEQ ID NO: 4. Murine prosaposin coding sequence is provided herein as SEQ ID NO: 1, and murine prosaposin amino acid sequence is provided herein as SEQ ID NO: 3.

In some embodiments, a nucleic acid encoding prosaposin can comprise one of SEQ ID NOs: 1 or 2, or a sequence that is at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% identical to the sequence of one of SEQ ID NOs: 1 or 2 that maintains the same function. In some embodiments, the prosaposin can have amino acid sequence of SEQ ID Nos 3 or 4, or a sequence that is at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% identical to the sequence of one of SEQ ID NOs: 3 or 4 that maintains the same function. As used herein, “function” of prosaposin or saposin includes one or more of degrading apoptotic vesicles from tumor cells (e.g., using a calcein release assay as described herein), and promoting cross-presentation of membrane-associated tumor antigen (e.g., assayed as described in the Examples described herein; such “function” can be, for example, at least 25%, at least 30%, at least 40%, at least 50%, at least 60%, at least 70%, at least 80% at least 90% or more relative to the function of a prosaposin of SEQ ID Nos 3 or 4.

Mus musculus prosaposin, complete CDS
GenBank: U57999.1
>U57999 Mus musculus prosaposin (psap\SGP-1) gene, complete cds
(SEQ ID NO: 1)
ATGTACGCCCTCGCCCTCTTCGCCAGCCTTCTGGCCACCGCTCTGACCAGCCCTGTCCAAGACCCGAAGACATGC
TCTGGGGGCTCAGCAGTGCTGTGCAGAGATGTGAAGACGGCGGTGGACTGTGGGGCCGTGAAGCACTGCCAGCAG
ATGGTCTGGAGCAAGCCCACAGCGAAATCCCTTCCTTGCGACATATGCAAAACTGTTGTCACCGAAGCTGGGAAC
TTGCTGAAAGATAATGCTACGCAGGAGGAGATCCTTCATTACCTGGAGAAGACCTGTGAGTGGATTCATGACTCC
AGCCTGTCGGCCTCGTGCAAGGAGGTGGTTGACTCTTACCTGCCTGTCATCCTGGACATGATTAAGGGCGAGATG
AGCAACCCTGGGGAAGTGTGCTCTGCGCTCAACCTCTGCCAGTCCCTTCAGGAGTACTTGGCCGAGCAAAACCAG
AAACAGCTTGAGTCCAACAAGATCCCGGAGGTGGACATGGCCCGTGTGGTTGCCCCCTTCATGTCCAACATCCCT
CTCCTGCTGTACCCTCAGGATCACCCCCGCAGCCAGCCCCAACCTAAGGCTAACGAGGACGTCTGCCAGGACTGT
ATGAAGCTGGTGTCTGATGTCCAGACTGCTGTGAAGACCAACTCCAGCTTTATCCAGGGCTTCGTGGACCACGTG
AAGGAGGATTGTGACCGCTTGGGGCCAGGCGTGTCTGACATATGCAAGAACTACGTGGACCAGTATTCCGAGGTC
TGTGTCCAGATGTTGATGCACATGCAGGATCAGCAACCCAAGGAAATCTGTGTGCTGGCTGGCTTCTGTAATGAG
GTCAAGAGAGTGCCAATGAAGACTCTGGTCCCTGCCACCGAGACCATTAAGAACATCCTCCCTGCCCTGGAGATG
ATGGACCCCTATGAGCAGAATCTGGTCCAGGCCCACAATGTGATTTTATGCCAGACCTGTCAGTTTGTGATGAAT
AAGTTTTCTGAGCTGATTGTCAATAATGCCACTGAGGAGCTCCTAGTTAAAGGTTTGAGCAACGCATGCGCACTG
CTCCCCGATCCTGCCAGAACCAAGTGCCAGGAGGTGGTGGGAACATTTGGCCCCTCCCTGTTGGACATCTTTATC
CATGAGGTAAACCCCAGCTCTCTGTGCGGTGTGATCGGCCTCTGTGCTGCCCGCCCGGAGTTGGTGGAGGCACTT
GAGCAGCCTGCGCCAGCCATTGTATCTGCACTGCTCAAAGAGCCCACACCGCCAAAGCAGCCCGCACAGCCCAAG
CAGTCGGCATTGCCCGCCCATGTGCCTCCTCAGAAGAATGGTGGGTTCTGTGAGGTGTGCAAGAAACCTGTCCTC
TATTTGGAACATAACCTGGAGAAAAACAGCACCAAGGAGGAAATCCTGGCCGCACTTGAGAAGGGCTGCAGCTTC
CTGCCAGACCCTTACCAGAAGCAGTGCGATGACTTTGTGGCTGAGTATGAGCCCTTGCTATTGGAGATCCTCGTG
GAAGTGATGGATCCTGGATTTGTGTGCTCGAAAATTGGAGTTTGCCCTTCTGCCTATAAGCTGCTGCTGGGAACC
GAGAAGTGTGTCTGGGGCCCTAGCTACTGGTGTCAGAACATGGAGACTGCCGCCCGATGCAATGCTGTCGATCAT
TGCAAACGCCATGTGTGGAACTAG
Homo sapiens prosaposin, complete CDS
GENBANK: BC001503.2
>BC001503.2: 6-1580 Homo sapiens prosaposin
(SEQ ID NO: 2)
ATGTACGCCCTCTTCCTCCTGGCCAGCCTCCTGGGCGCGGCTCTAGCCGGCCCGGTCCTTGGACTGAAAGAATGC
ACCAGGGGCTCGGCAGTGTGGTGCCAGAATGTGAAGACGGCGTCCGACTGCGGGGCAGTGAAGCACTGCCTGCAG
ACCGTTTGGAACAAGCCAACAGTGAAATCCCTTCCCTGCGACATATGCAAAGACGTTGTCACCGCAGCTGGTGAT
ATGCTGAAGGACAATGCCACTGAGGAGGAGATCCTTGTTTACTTGGAGAAGACCTGTGACTGGCTTCCGAAACCG
AACATGTCTGCTTCATGCAAGGAGATAGTGGACTCCTACCTCCCTGTCATCCTGGACATCATTAAAGGAGAAATG
AGCCGTCCTGGGGAGGTGTGCTCTGCTCTCAACCTCTGCGAGTCTCTCCAGAAGCACCTAGCAGAGCTGAATCAC
CAGAAGCAGCTGGAGTCCAATAAGATCCCAGAGCTGGACATGACTGAGGTGGTGGCCCCCTTCATGGCCAACATC
CCTCTCCTCCTCTACCCTCAGGACGGCCCCCGCAGCAAGCCCCAGCCAAAGGATAATGGGGACGTTTGCCAGGAC
TGCATTCAGATGGTGACTGACATCCAGACTGCTGTACGGACCAACTCCACCTTTGTCCAGGCCTTGGTGGAACAT
GTCAAGGAGGAGTGTGACCGCCTGGGCCCTGGCATGGCCGACATATGCAAGAACTATATCAGCCAGTATTCTGAA
ATTGCTATCCAGATGATGATGCACATGCAACCCAAGGAGATCTGTGCGCTGGTTGGGTTCTGTGATGAGGTGAAA
GAGATGCCCATGCAGACTCTGGTCCCCGCCAAAGTGGCCTCCAAGAATGTCATCCCTGCCCTGGAACTGGTGGAG
CCCATTAAGAAGCACGAGGTCCCAGCAAAGTCTGATGTTTACTGTGAGGTGTGTGAATTCCTGGTGAAGGAGGTG
ACCAAGCTGATTGACAACAACAAGACTGAGAAAGAAATACTCGACGCTTTTGACAAAATGTGCTCGAAGCTGCCG
AAGTCCCTGTCGGAAGAGTGCCAGGAGGTGGTGGACACGTACGGCAGCTCCATCCTGTCCATCCTGCTGGAGGAG
GTCAGCCCTGAGCTGGTGTGCAGCATGCTGCACCTCTGCTCTGGCACGCGGCTGCCTGCACTGACCGTTCACGTG
ACTCAGCCAAAGGACGGTGGCTTCTGCGAAGTGTGCAAGAAGCTGGTGGGTTATTTGGATCGCAACCTGGAGAAA
AACAGCACCAAGCAGGAGATCCTGGCTGCTCTTGAGAAAGGCTGCAGCTTCCTGCCAGACCCTTACCAGAAGCAG
TGTGATCAGTTTGTGGCAGAGTACGAGCCCGTGCTGATCGAGATCCTGGTGGAGGTGATGGATCCTTCCTTCGTG
TGCTTGAAAATTGGAGCCTGCCCCTCGGCCCATAAGCCCTTGTTGGGAACTGAGAAGTGTATATGGGGCCCAAGC
TACTGGTGCCAGAACACAGAGACAGCAGCCCAGTGCAATGCTGTCGAGCATTGCAAACGCCATGTGTGGAACTAG
MUS MUSCULUS PROSAPOSIN AA SEQUENCE
GenBank: AAB02695.1
>AAB02695.1 prosaposin [Mus musculus]
(SEQ ID NO: 3)
MYALALFASLLATALTSPVQDPKTCSGGSAVLCRDVKTAVDCGAVKHCQQMVWSKPTAKSLPCDICKTVVTEAGN
LLKDNATQEEILHYLEKTCEWIHDSSLSASCKEVVDSYLPVILDMIKGEMSNPGEVCSALNLCQSLQEYLAEQNQ
KQLESNKIPEVDMARVVAPFMSNIPLLLYPQDHPRSQPQPKANEDVCQDCMKLVSDVQTAVKTNSSFIQGFVDHV
KEDCDRLGPGVSDICKNYVDQYSEVCVQMLMHMQDQQPKEICVLAGFCNEVKRVPMKTLVPATETIKNILPALEM
MDPYEQNLVQAHNVILCQTCQFVMNKFSELIVNNATEELLVKGLSNACALLPDPARTKCQEVVGTFGPSLLDIFI
HEVNPSSLCGVIGLCAARPELVEALEQPAPAIVSALLKEPTPPKQPAQPKQSALPAHVPPQKNGGFCEVCKKPVL
YLEHNLEKNSTKEEILAALEKGCSFLPDPYQKQCDDFVAEYEPLLLEILVEVMDPGFVCSKIGVCPSAYKLLLGT
EKCVWGPSYWCQNMETAARCNAVDHCKRHVWN
HOMO SAPIENS PROSAPOSIN AA SEQUENCE
GenBank: KAI4076306.1
>KAI4076306.1 prosaposin [Homo sapiens]
(SEQ ID NO: 4)
MYALFLLASLLGAALAGPVLGLKECTRGSAVWCQNVKTASDCGAVKHCLQTVWNKPTVKSLPCDICKDVVTAAGD
MLKDNATEEEILVYLEKTCDWLPKPNMSASCKEIVDSYLPVILDIIKGEMSRPGEVCSALNLCESLQKHLAELNH
QKQLESNKIPELDMTEVVAPFMANIPLLLYPQDGPRSKPQPKDNGDVCQDCIQMVTDIQTAVRTNSTFVQALVEH
VKEECDRLGPGMADICKNYISQYSEIAIQMMMHMSLQQPKEICALVGFCDEVKEMPMQTLVPAKVASKNVIPALE
LVEPIKKHEVPAKSDVYCEVCEFLVKEVTKLIDNNKTEKEILDAFDKMCSKLPKSLSEECQEVVDTYGSSILSIL
LEEVSPELVCSMLHLCSGTRLPALTVHVTQPKDGGFCEVCKKLVGYLDRNLEKNSTKQEILAALEKGCSFLPDPY
QKQCDQFVAEYEPVLIEILVEVMDPSFVCLKIGACPSAHKPLLGTEKCIWGPSYWCQNTETAAQCNAVEHCKRHV
WN

### Dendritic Cell Targeting Moieties

In some embodiments, a dendritic cell targeting moiety for targeting a saposin or prosaposin to dendritic cells comprises an antibody or antigen binding fragment thereof. A variety of suitable antibody reagent formats are known in the art, such as complete antibodies, e.g., an IgG, or modified forms or fragments of such antibodies, including, as non-limiting examples, single chain antibodies, heterodimers of antibody heavy chains and/or light chains, an Fv fragment (e.g., single chain Fv (scFv), a disulfide bonded Fv), a Fab fragment, a Fab′ fragment, a F(ab′)2 fragment), a single variable domain (e.g., VH, VL, VHH), a dAb, and modified versions of any of the foregoing (e.g., modified by the covalent attachment of polyalkylene glycol (e.g., polyethylene glycol, polypropylene glycol, polybutylene glycol) or other suitable polymer). Antibody reagents or constructs can, if desired, be linked to an antibody Fc region, comprising one or both of CH2 and CH3 domains, and optionally, a hinge region. Such linkage can provide benefits such as increased serum half-life or promotion of effector function(s). Alternatively, antibody reagents or constructs can be fused to a carrier such as serum albumin to promote increased serum half-life.

Examples of antibodies that target a saposin or prosaposin to dendritic cells include, but are not limited to in mice: DEC-205, CD11c, Clec9a, MHC-II, SIRP1alpha, CD103, Flt3, CX3CR1, DC-SIGN (CD209), Langerin (CD207), XCR1, mannose receptor (CD206), DCIR, and Dectin. Examples of antibodies that target a saposin or prosaposin to dendritic cells include, but are not limited to in human: CD11c, HLA-DR, CD141, Clec9a, CD1c, SIRP1alpha, Flt3, CD1a, DC-SIGN (CD209), Langerin (CD207), CD103, XCR1, CD14, mannose receptor (CD206), DCIR, and Dectin.

DEC-205 is also known more commonly as LY75 or CD205 and can be used interchangeably. DEC-205 is an endocytic receptor that is expressed at high levels by cortical thymic epithelial cells and by dendritic cell (DC) subsets, including the splenic CD8+DC population that is responsible for cross-presentation of apoptotic cell-derived antigens. Antigen endocytosed via DEC-205 enters the MHC class I and MHC class II antigen presentation pathways and is subsequently presented to both CD4+ and CD8+ T cells.

DEC-205 can be found inMus musculus and Homo sapiens, as well as other mammals. The sequences for DEC-205 in Mus musculus and Homo sapiens can be found in the following paragraphs:

Homo sapiens mRNA(NM_002349.4, NCBI Gene ID: 4065), SEQ ID NO: 15:
aggccgcgct cagcaggcgg ggcgggagcc gcgtgcgccc gaggacccgg
ccggaaggcttgcgccagct caggatgagg acaggctggg cgacccctcg ccgcccggcg
gggctcctcatgctgctctt ctggttcttc gatctcgcgg agccctctgg ccgcgcagct
aatgaccccttcaccatcgt ccatggaaat acgggcaagt gcatcaagcc agtgtatggc
tggatagtagcagacgactg tgatgaaact gaggacaagt tatggaagtg ggtgtcccag
catcggctctttcatttgca ctcccaaaag tgccttggcc tcgatattac caaatcggta
aatgagctgagaatgttcag ctgtgactcc agtgccatgc tgtggtggaa atgtgagcac
cactctctgtacggagctgc ccggtaccgg ctggctctga aggatggaca tggcacagca
atctcaaatgcatctgatgt ctggaagaaa aactcttatg ggagaccttg tgaatttcca
ccttatcatgagatctatac cagagatggg ggaggctcag aggaaagcct ttgtgaccag
ttcttaattgatgggacctg gcatcatgat tgcattcttg atgaagatca tagtgggcca
tggtgtgccaccaccttaaa ttatgaatat gaccgaaagt ggggcatctg cttaaagcct
gaaaacggttgtgaagataa ttgggaaaag aacgagcagt ttggaagttg ctaccaattt
aatactcagacggctctttc ttggaaagaa gcttatgttt catgtcagaa tcaaggagct
gatttactgagcatcaacag tgctgctgaa ttaacttacc ttaaagaaaa agaaggcatt
gctaagattttctggattgg tttaaatcag ctatactctg ctagaggctg ggaatggtca
gaccacaaaccattaaactt tctcaactgg gatccagaca ggcccagtgc acctactata
ggtggctccagctgtgcaag aatggatgct gagtctggtc tgtggcagag cttttcctgt
gaagctcaactgccctatgt ctgcaggaaa ccattaaata atacagtgga gttaacagat
gtctggacatactcagatac ccgctgtgat gcaggctggc tgccaaataa tggattttgc
tatctgctggtaaatgaaag taattcctgg gataaggcac atgcgaaatg caaagccttc
agtagtgacctaatcagcat tcattctcta gcagatgtgg aggtggttgt cacaaaactc
cataatgaggatatcaaaga agaagtgtgg ataggcctta agaacataaa cataccaact
ttatttcagtggtcagatgg tactgaagtt actctaacat attgggatga gaatgagcca
aatgttccctacaataagac gcccaactgt gtttcctact taggagagct aggtcagtgg
aaagtccaatcatgtgagga gaaactaaaa tatgtatgca agagaaaggg agaaaaactg
aatgacgcaagttctgataa gatgtgtcct ccagatgagg gctggaagag acatggagaa
acctgttacaagatttatga ggatgaggtc ccttttggaa caaactgcaa tctgactatc
actagcagatttgagcaaga atacctaaat gatttgatga aaaagtatga taaatctcta
agaaaatacttctggactgg cctgagagat gtagattctt gtggagagta taactgggca
actgttggtggaagaaggcg ggctgtaacc ttttccaact ggaattttct tgagccagct
tccccgggcggctgcgtggc tatgtctact ggaaagtctg ttggaaagtg ggaggtgaag
gactgcagaagcttcaaagc actttcaatt tgcaagaaaa tgagtggacc ccttgggcct
gaagaagcatcccctaagcc tgatgacccc tgtcctgaag gctggcagag tttccccgca
agtctttcttgttataaggt attccatgca gaaagaattg taagaaagag gaactgggaa
gaagctgaacgattctgcca agcccttgga gcacaccttt ctagcttcag ccatgtggat
gaaataaaggaatttcttca ctttttaacg gaccagttca gtggccagca ttggctgtgg
attggtttgaataaaaggag cccagattta caaggatcct ggcaatggag tgatcgtaca
ccagtgtctactattatcat gccaaatgag tttcagcagg attatgacat cagagactgt
gctgctgtcaaggtatttca taggccatgg cgaagaggct ggcatttcta tgatgataga
gaatttatttatttgaggcc ttttgcttgt gatacaaaac ttgaatgggt gtgccaaatt
ccaaaaggccgtactccaaa aacaccagac tggtacaatc cagaccgtgc tggaattcat
ggacctccacttataattga aggaagtgaa tattggtttg ttgctgatct tcacctaaac
tatgaagaagccgtcctgta ctgtgccagc aatcacagct ttcttgcaac tataacatct
tttgtgggactaaaagccat caaaaacaaa atagcaaata tatctggtga tggacagaag
tggtggataagaattagcga gtggccaata gatgatcatt ttacatactc acgatatcca
tggcaccgctttcctgtgac atttggagag gaatgcttgt acatgtctgc caagacttgg
cttatcgacttaggtaaacc aacagactgt agtaccaagt tgcccttcat ctgtgaaaaa
tataatgtttcttcgttaga gaaatacagc ccagattctg cagctaaagt gcaatgttct
gagcaatggattccttttca gaataagtgt tttctaaaga tcaaacccgt gtctctcaca
ttttctcaagcaagcgatac ctgtcactcc tatggtggca cccttccttc agtgttgagc
cagattgaacaagactttat tacatccttg cttccggata tggaagctac tttatggatt
ggtttgcgctggactgccta tgaaaagata aacaaatgga cagataacag agagctgacg
tacagtaactttcacccatt attggttagt gggaggctga gaataccaga aaattttttt
gaggaagagtctcgctacca ctgtgcccta atactcaacc tccaaaaatc accgtttact
gggacgtggaattttacatc ctgcagtgaa cgccactttg tgtctctctg tcagaaatat
tcagaagttaaaagcagaca gacgttgcag aatgcttcag aaactgtaaa gtatctaaat
aatctgtacaaaataatccc aaagactctg acttggcaca gtgctaaaag ggagtgtctg
aaaagtaacatgcagctggt gagcatcacg gacccttacc agcaggcatt cctcagtgtg
caggcgctccttcacaactc ttccttatgg atcggactct tcagtcaaga tgatgaactc
aactttggttggtcagatgg gaaacgtctt cattttagtc gctgggctga aactaatggg
caactcgaagactgtgtagt attagacact gatggattct ggaaaacagt tgattgcaat
gacaatcaaccaggtgctat ttgctactat tcaggaaatg agactgaaaa agaggtcaaa
ccagttgacagtgttaaatg tccatctcct gttctaaata ctccgtggat accatttcag
aactgttgctacaatttcat aataacaaag aataggcata tggcaacaac acaggatgaa
gttcatactaaatgccagaa actgaatcca aaatcacata ttctgagtat tcgagatgaa
aaggagaataactttgttct tgagcaactg ctgtacttca attatatggc ttcatgggtc
atgttaggaataacttatag aaataagtct cttatgtggt ttgataagac cccactgtca
tatacacattggagagcagg aagaccaact ataaaaaatg agaagttttt ggctggttta
agtactgacggcttctggga tattcaaacc tttaaagtta ttgaagaagc agtttatttt
caccagcacagcattcttgc ttgtaaaatt gaaatggttg actacaaaga agaatataat
actacactgccacagtttat gccatatgaa gatggtattt acagtgttat tcaaaaaaag
gtaacatggtatgaagcatt aaacatgtgt tctcaaagtg gaggtcactt ggcaagcgtt
cacaaccaaaatggccagct ctttctggaa gatattgtaa aacgtgatgg atttccacta
tgggttgggctctcaagtca tgatggaagt gaatcaagtt ttgaatggtc tgatggtagt
acatttgactatatcccatg gaaaggccaa acatctcctg gaaattgtgt tctcttggat
ccaaaaggaacttggaaaca tgaaaaatgc aactctgtta aggatggtgc tatttgttat
aaacctacaaaatctaaaaa gctgtcccgt cttacatatt catcaagatg tccagcagca
aaagagaatgggtcacggtg gatccagtac aagggtcact gttacaagtc tgatcaggca
ttgcacagtttttcagaggc caaaaaattg tgttcaaaac atgatcactc tgcaactatc
gtttccataaaagatgaaga tgagaataaa tttgtgagca gactgatgag ggaaaataat
aacattaccatgagagtttg gcttggatta tctcaacatt ctgttgacca gtcttggagt
tggttagatggatcagaagt gacatttgtc aaatgggaaa ataaaagtaa gagtggtgtt
ggaagatgtagcatgttgat agcttcaaat gaaacttgga aaaaagttga atgtgaacat
ggttttggaagagttgtctg caaagtgcct ctgggccctg attacacagc aatagctatc
atagttgccacactaagtat cttagttctc atgggcggac tgatttggtt cctcttccaa
aggcaccgtttgcacctggc gggtttctca tcagttcgat atgcacaagg agtgaatgaa
gatgagattatgcttccttc tttccatgac taaattcttc taaaagtttt ctaatttgca
ctaatgtgttatgagaaatt agtcacttaa aatgtcccag tgtcagtatt tactctgctc
caaagtagaactcttaaata ctttttcagt tgtttagatc ttaggcatgt gctggtatcc
acagttaattccctgctaaa tgccatgttt atcaccctaa ttaatagaat ggaggggact
ccaaagctggaactgaagtc caaattgttt gtacagtaat atgtttaatg ttcattttct
ctgtatgaatgtgattggta actaggatat gtatatttta atagaatttt taacaaaact
tcttagaaaattaaaatagg catattacta ggtgacatgt ctacttttta atttttaaga
gcatccggccaaatgcaaaa ttagtacctc aaagtaaaaa ttgaactgta aactctatca
gcattgtttcaaaatagtca tttttagcac tggggaaaaa taaacaataa gacatgctta
ctttttaatttttatttttt tgagactgag tctctctctg ttgcccaggc tggagtacaa
tggcgtgatctcggctcact gcaaatctcc gcctcccagg ttcaagcgat tctcctgcct
cagcctcctgagtagctggg attacaggca actgccacca tgcccggcta atttttgtat
ttttagtagagatggggttt caccatgttg gccaggctgg tctcgaactc gtgaccgcag
gtgatcctcccgcctcggcc toccaaagtg ctgggattac aggcatgagc caccgcgcct
ggcctctgcttactttttat atagcaaaat gattcctctt ggcaagatgt ttcttatatt
attccaaagttatttcatac cattattatg taaatatgaa gagttttttt ctgtttataa
ttgtttataaaacaatgact tttaaagatt tagtgcttaa cattttccca agtgtgggaa
cattatttttagattgagta ggtaccttgt agcagtgtgc tttgcatttt ctgatgtatt
acatgactgtttcttttgta aagagaatca actaggtatt taagactgat aattttacaa
tttatatgcttcacatagca tgtcaacttt tgactaagaa ttttgtttta cttttttaac
atgtgttaaacagagaaagg gtccatgaag gaaagtgtat gagttgcatt tgtaaaaatg
agactttttcagtggaactc taaaccttgt gatgactact aacaaatgta aaattatgag
tgattaagaaaacattgctt tgtggttatc actttaagtt ttgacaccta gattatagtc
ttagtaatagcatccactgg aaaaggtgaa aatgttttat tcggcattta acttacattt
gtactttatttttgtataaa atccatagat ttattttaca tttagagtat ttacactatg
ataaagttgtaaataatttt ctaagacagt ttttatatag tctacagttg tcctgatttc
ttattgaatttgttagacta gttctcttgt cctgtgatct gtgtacaatt ttagtcacta
agactttcctccaagaacta agccaacttg atgtgaaaag cacagctgta tataatggtg
atgtcataataaagttgttt tatcttttaa gtaaaagtaa aa
Mus musculus mRNA (NM_013825.4, NCBI Gene ID: 17076), SEQ ID NO: 16:
accgctgaag ccggtgcggg caggagaggc ttgggactgg agatgcggac
gggccgggtgaccccgggcc tggcggcggg gctactcctg ctgttgctgc ggtccttcgg
gcttgtggagccttctgaga gctcaggtaa tgatccattc accatcgtcc atgaaaacac
tggcaagtgcatccagccgc tgtctgactg ggtagtggcc caggactgta gcggaactaa
caacatgttgtggaagtggg tgtcccagca ccgcctcttt cacctggaat cccagaagtg
cctcggcctcgatattacca aagccacgga caacctgcga atgttcagct gtgactccac
cgtcatgctgtggtggaaat gtgagcacca ttcgctgtac accgctgccc agtacaggct
agctctgaaagatggatatg ccgtagccaa tacgaataca tctgatgtct ggaagaaggg
aggctccgaggaaaaccttt gtgcccagcc ttatcatgag atatacacca gagatgggaa
ttcctacgggagaccttgtg aattcccttt cttgattggt gagacatggt accatgactg
cattcatgatgaagatcata gtgggccatg gtgtgccact accctaagtt atgaatatga
tcaaaagtggggcatctgcc tactaccaga aagtggctgt gaaggtaact gggaaaagaa
tgagcagattggaagttgct accaatttaa taatcaggaa attctgtctt ggaaagaagc
ttatgtttcctgtcagaacc aaggagctga cttactgagc atccacagtg ctgccgaatt
agcctacattacgggaaaag aggacattgc tagacttgtt tggcttggac tgaatcagct
ctattctgcgagaggttggg aatggtcaga cttcaggcca ctcaaatttc ttaactggga
tccaggcacgcccgttgcac ctgtgattgg tgggtcaagc tgtgccagaa tggacacaga
gtccgggctgtggcaaagtg tttcctgtga atctcagcag ccttacgtct gcaagaagcc
actgaacaacacgctggagc tcccagatgt ttggacttac acagataccc actgccatgt
gggctggctgccaaataatg ggttttgcta tctgctggcg aatgaaagta gttcctggga
tgcagcacatttgaaatgca aagccttcgg tgcagacctc atcagcatgc actccttagc
agatgtggaggtggttgtca cgaaactcca taatggggat gtcaaaaaag aaatatggac
aggccttaaaaacacaaaca gccctgcttt gttccagtgg tcggacggaa cggaagttac
tctaacgtactggaatgaga atgagccgag tgttcccttc aacaagactc ccaactgtgt
ttcctatttaggaaagttag gtcagtggaa agtccagtcc tgtgagaaga aactcagata
tgtatgcaagaaaaagggag aaataactaa ggatgcagag tcggataagc tgtgtccgcc
agacgagggctggaagagac atggagaaac ctgttacaag atttatgaga aagaggcccc
tttcggaacgaactgcaacc tgaccatcac tagcaggttc gagcaggaat tottgaatta
tatgatgaagaactatgata agtcccttcg gaagtacttc tggactggcc tgagagaccc
tgactctcgaggagaataca gttgggccgt tgctcaggga gtaaagcagg ctgtgacctt
ttccaactggaattttcttg aaccggcgtc tccaggcggg tgcgtggcta tgtctactgg
aaagactcttggcaagtggg aagtgaagaa ctgcagaagc ttccgtgctc tttcaatatg
caagaaagtgagcgaacccc aggagcctga agaagcagcc cccaagcccg acgacccctg
tcctgaaggctggcacactt tcccctccag cctttcttgt tataaggtgt tccatataga
aagaatcgtaagaaagagga actgggaaga agccgaaagg ttctgccaag cccttggagc
tcacctacccagcttcagtc gtagagagga aattaaggac tttgtgcatt tgttaaagga
ccagttcagtgggcagcgtt ggttgtggat tggtctgaat aagagaagcc ctgatttaca
agggtcctggcagtggagtg accggacacc agtgtctgct gtgatgatgg agccggagtt
tcaacaggattttgacatca gagactgtgc tgccatcaag gtccttgatg taccttggcg
aagagtctggcatctctatg aggacaagga ctatgcttac tggaaacctt ttgcttgtga
tgccaagcttgagtgggtgt gccagattcc aaaaggtagc actccccaga tgccagactg
gtataatccagagcgcactg gaattcatgg gcccccagtt ataattgaag gaagtgaata
ctggtttgttgctgatcccc acttaaacta cgaagaagcc gtcttatact gtgctagcaa
tcacagctttcttgccacga taacatcgtt cacaggacta aaagctatca aaaacaaact
agcaaatatttctggcgagg aacagaagtg gtgggtgaaa acgagtgaga atccaattga
tcgttactttctaggctcgc gccgccgcct gtggcaccat ttccccatga cgtttggaga
tgaatgtttgcacatgtcag ccaagacgtg gcttgttgac ttaagtaaac gagcggactg
taatgccaagttgcccttca tctgtgaaag atacaatgtc tcttcattag agaaatacag
cccagatcctgcagccaaag tacagtgcac tgagaagtgg attccttttc aaaataagtg
cttcctaaaggtcaactctg ggcccgttac gttttctcaa gcaagcggca tttgtcattc
ctacggcggcacccttcctt ccgtgctgag ccggggtgaa caagatttca ttatatcctt
gcttcctgaaatggaagcta gtctatggat tggtctgcgc tggactgcct acgaaaggat
aaacagatggacagacaaca gagagctgac ctacagcaac tttcacccac tgctggtcgg
tcggaggctgagcataccaa cgaatttctt tgatgatgag tcccacttcc actgcgcctt
gattcttaatctcaaaaagt caccgcttac tgggacctgg aattttactt cctgttcaga
acgacactctctgtctctct gtcaaaaata ctcagagact gaagacggac agccctggga
gaacacttcaaaaacagtga agtatctaaa taacctatac aaaatcatct cgaagcccct
gacgtggcacggcgctctga aggagtgcat gaaagagaag atgaggttgg tgagcatcac
agacccttaccagcaggcct tcctcgcagt gcaggccacc ctgcgcaaca gctccttctg
gatcggactctccagtcaag atgatgaact caactttggt tggtcagatg ggaaacgtct
tcaatttagtaactgggctg gaagcaatga gcaacttgat gactgcgtga tattagacac
agatggattctggaaaacag ctgactgtga tgataaccag cctggcgcca tttgctacta
tccaggaaatgagactgagg aggaggtcag agcactggac actgctaaat gcccgtctcc
tgtacagagcaccccatgga taccattcca gaactcctgc tacaatttca tgattaccaa
caacaggcataagacagtca caccggagga agtgcagtcc acgtgcgaga agctgcattc
gaaagcacacagtctgagca ttcggaatga ggaggagaat acctttgttg tggaacagct
tctgtacttcaattatattg cctcatgggt catgttagga ataacctatg aaaacaattc
tttgatgtggtttgataaaa ctgcattgtc ctacacacac tggagaacgg gaagaccaac
tgtgaaaaatggcaaatttt tggctggtct aagtactgat ggattctggg atattcagtc
tttcaatgttattgaagaaa cacttcattt ttaccagcac agtatttctg cttgtaaaat
tgaaatggttgactatgagg acaaacacaa tggcaccctg ccacagttca ttccatataa
ggacggcgtctacagcgtta ttcagaagaa ggtgacgtgg tatgaagcat tgaacgcgtg
ctctcaaagtgggggagagt tggccagtgt tcacaaccca aatgggaagc tctttctgga
agacattgtgaaccgtgacg gattccctct ctgggttggg ctctcaagtc atgatggaag
cgaatcgagtttcgaatggt ccgatggcag agcatttgac tatgtcccat ggcagagcct
acaatctcccggagactgtg tcgtcttata tccaaaagga atttggagac gtgaaaaatg
cctgtctgttaaggatggtg ctatttgtta caagcctaca aaagataaaa agctgatctt
tcatgtaaaatcatcaaaat gtccagtggc aaagagggat ggtccccagt gggtccagta
tgggggccactgttacgctt cggaccaggt actgcacagc ttctcagagg ccaaacaagt
gtgtcaagagcttgatcatt cggcaactgt tgtcaccata gcagatgaaa atgagaataa
gtttgtgagcagactgatga gggagaacta taatattact atgagagttt ggcttggcct
gtctcagcattcactcgatc agtcttggag ttggctcgat ggattagatg tgacatttgt
caaatgggaaaataaaacta aggatggtga tgggaaatgt agcattttaa tagcttcaaa
tgaaacctggagaaaagtcc attgctcacg tggctatgca agagctgtct gcaaaattcc
tctgagcccggactacacag gcatagccat cctgtttgcc gtgctgtgcc tcttagggct
catcagcttggcgatttggt tcctcttgca acgatcccat atccgctgga ccggcttctc
ctcggttcggtatgaacatg gaaccaacga agacgaggtg atgctccctt ctttccacga
ctaacttcttccaagagttt gttcattggc cctggatgtt gcggaagcaa gatgagccac
ttagcactgcatccccccaa tcagtgtcta actttgtttc taagtagaga agtcttagag
atgtattcagcgggcgggag gggagagttg ctcagctgat gccctaacgg ccatgctaat
gagtcagttatcagagcaga gagacttaaa gccaaaatgg atattcaggt tgttcacagg
gggataaatttagctaatgt gaattttctc tgtaaaaatg cagttgctaa atagatacgt
acatattgtaatagcattgt aacagcctca tagaaaatta aaactgtcag tacaagtagg
ctccacatttaccttttatg ggcatctgac caaatgcaaa gttagtaact ccagcaaaaa
ctcaactataaataagatca atgatgcttc aaaatagcct tttaaggcac tggggaaaaa
atacaaaataaggcatattt tttatacaga aaacagagcc ccttggaaga tgtttcttat
actatttcaaagatatgtat actggtatta attaattgtt acatttctgt ttttcattgc
ttatattcagtgagttggtt tttgtcttta acttaaagtt aatgtttcct aaagttggga
acgttgctgttaagttgcct gggcgtgctg tatcagcatg tctgcgtttg atgattttct
tttttacaaggacccaatta cagctgagac ttcagctttt acagttcctg tctttcacat
aggagatcagctttctgttg gtggtggctg gggtttttgg ttttgttttg ttgtttttgt
ttttgttttgttttgttttt tgaaacatgg tttctttgtg catcatagcc ctggctgtcc
tggactgactttgtagacca ggctagccac taattcagag atcccccgcc tctgccttgg
gagtgctgggatcaaagact gtgctggcac caccccagca agcaggcagg gtatcacctt
ttgatgaatgtattatgttg ttgccttttt acttgagcga aacataaaag tgatctttga
ataatgttgtttgctacttc tgtatttgta aacaatcacc gtccctaaga caatgaaact
cagaactctgtgattcctgt ggaaatattt aaaactgaaa tgattagtga cacactattt
tgttgatgtaatttccattt gaacatctgg aatatacttt tactagtagt gtcaaaataa
atatggtgactgttttattc agtgtttcat atttatactt tagagtattt ttgtacaaga
tccagatttattttgcttga ggtgtttatg ctatcataaa gttgtaaata attttctaag
actgttttaatgcaatctac agttttccta aaatattttt cttgaggcct cttttaacta
tcactaggagtttcttcaaa aaactaaatc aataccatgt gaccacagtt gtgatcatgg
ctgcttccgtggctgatcct tgcatgtgaa agcatctcta aattaggttt gattgtatcc
catgtgtagtatgaagagaa ggtgtccagc tattgcgagt taaggtattt taagactttg
tgagcactttttaaagaata aatctttgta tgtctaaatt a
Homo sapiens protein (NP_002340.2, NCBI Gene ID: 4065), SEQ ID NO: 17:
mrtgwatprr pagllmllfw ffdlaepsgr aandpftivh gntgkcikpv ygwivaddcd
etedklwkwv sqhrlfhlhs qkclglditk svnelrmfsc dssamlwwkc ehhslygaar
yrlalkdghg taisnasdvw kkggseeslc dqpyheiytr dgnsygrpce fpflidgtwh
hdcildedhs gpwcattlny eydrkwgicl kpengcednw ekneqfgscy qfntqtalsw
keayvscqnq gadllsinsa aeltylkeke giakifwigl nqlysargwe wsdhkplnfl
nwdpdrpsap tiggsscarm daesglwqsf sceaqlpyvc rkplnntvel tdvwtysdtr
cdagwlpnng fcyllvnesn swdkahakck afssdlisih sladvevvvt klhnedikee
vwiglknini ptlfqwsdgt evtltywden epnvpynktp ncvsylgelg qwkvqsceek
lkyvckrkge klndassdkm cppdegwkrh getcykiyed evpfgtncnl titsrfeqey
lndlmkkydk slrkyfwtgl rdvdscgeyn watvggrrra vtfsnwnfle paspggcvam
stgksvgkwe vkdcrsfkal sickkmsgpl gpeeaspkpd dpcpegwqsf paslscykvf
haerivrkrn weeaerfcqa lgahlssfsh vdeikeflhf ltdqfsgqhw lwiglnkrsp
dlqgswqwsd rtpvstiimp nefqqdydir dcaavkvfhr pwrrgwhfyd drefiylrpf
acdtklewvc qipkgrtpkt pdwynpdrag ihgppliieg seywfvadlh lnyeeavlyc
asnhsflati tsfvglkaik nkianisgdg qkwwirisew piddhftysr ypwhrfpvtf
geeclymsak twlidlgkpt dcstklpfic ekynvsslek yspdsaakvq cseqwipfqn
kcflkikpvs ltfsqasdtc hsyggtlpsv lsqieqdfit sllpdmeatl wiglrwtaye
kinkwtdnre 1tysnfhpll vsgrlripen ffeeesryhc alilnlqksp ftgtwnftsc
serhfvslcq kysevksrqt lqnasetvky Innlykiipk tltwhsakre clksnmqlvs
itdpyqqafl svqallhnss lwiglfsqdd elnfgwsdgk rlhfsrwaet ngqledcvvl
dtdgfwktvd cndnqpgaic yysgneteke vkpvdsvkcp spvlntpwip fqnccynfii
tknrhmattq devhtkcqkl npkshilsir dekennfvle qllyfnymas wvmlgityrn
kslmwfdktp lsythwragr ptiknekfla glstdgfwdi qtfkvieeav yfhqhsilac
kiemvdykee ynttlpqfmp yedgiysviq kkvtwyealn mcsqsgghla svhnqngqlf
ledivkrdgf plwvglsshd gsessfewsd gstfdyipwk gqtspgncvl ldpkgtwkhe
kcnsvkdgai cykptkskkl srltyssrcp aakengsrwi qykghcyksd qalhsfseak
klcskhdhsa tivsikdede nkfvsrlmre nnnitmrvwl glsqhsvdqs wswldgsevt
fvkwenksks gvgrcsmlia snetwkkvec ehgfgrvvck vplgpdytai aiivatlsil
vlmggliwfl fqrhrlhlag fssvryaqgv nedeimlpsf hd
Mus musculus protein (NP_038853.2, NCBI Gene ID: 17076), SEQ ID NO: 18:
mrtgrvtpgl aagllllllr sfglvepses sgndpftivh entgkciqpl
sdwvvaqdcsgtnnmlwkwv sqhrlfhles qkclglditk atdnlrmfsc dstvmlwwkc
ehhslytaaqyrlalkdgya vantntsdvw kkggseenlc aqpyheiytr dgnsygrpce
fpfligetwyhdcihdedhs gpwcattlsy eydqkwgicl lpesgcegnw ekneqigscy
qfnnqeilswkeayvscqnq gadllsihsa aelayitgke diarlvwlgl nqlysargwe
wsdfrplkflnwdpgtpvap viggsscarm dtesglwqsv scesqqpyvc kkplnntlel
pdvwtytdthchvgwlpnng fcyllaness swdaahlkck afgadlismh sladvevvvt
klhngdvkkeiwtglkntns palfqwsdgt evtltywnen epsvpfnktp ncvsylgklg
qwkvqscekklryvckkkge itkdaesdkl cppdegwkrh getcykiyek eapfgtncnl
titsrfeqeflnymmknydk slrkyfwtgl rdpdsrgeys wavaqgvkqa vtfsnwnfle
paspggcvamstgktlgkwe vkncrsfral sickkvsepq epeeaapkpd dpcpegwhtf
psslscykvfhierivrkrn weeaerfcqa lgahlpsfsr reeikdfvhl lkdqfsgqrw
lwiglnkrspdlqgswqwsd rtpvsavmme pefqqdfdir dcaaikvldv pwrrvwhlye
dkdyaywkpfacdaklewvc qipkgstpqm pdwynpertg ihgppviieg seywfvadph
lnyeeavlycasnhsflati tsftglkaik nklanisgee qkwwvktsen pidryflgsr
rrlwhhfpmtfgdeclhmsa ktwlvdlskr adcnaklpfi cerynvssle kyspdpaakv
qctekwipfqnkcflkvnsg pvtfsqasgi chsyggtlps vlsrgeqdfi isllpemeas
lwiglrwtayerinrwtdnr eltysnfhpl lvgrrlsipt nffddeshfh calilnlkks
pltgtwnftscserhslslc qkysetedgq pwentsktvk ylnnlykiis kpltwhgalk
ecmkekmrlvsitdpyqqaf lavqatlrns sfwiglssqd delnfgwsdg krlqfsnwag
sneqlddcvildtdgfwkta dcddnqpgai cyypgnetee evraldtakc pspvqstpwi
pfqnscynfmitnnrhktvt peevqstcek lhskahslsi rneeentfvv eqllyfnyia
swvmlgityennslmwfdkt alsythwrtg rptvkngkfl aglstdgfwd iqsfnvieet
lhfyqhsisackiemvdyed khngtlpqfi pykdgvysvi qkkvtwyeal nacsqsggel
asvhnpngklfledivnrdg fplwvglssh dgsessfews dgrafdyvpw qslqspgdcv
vlypkgiwrrekclsvkdga icykptkdkk lifhvksskc pvakrdgpqw vqygghcyas
dqvlhsfseakqvcqeldhs atvvtiaden enkfvsrlmr enynitmrvw lglsqhsldq
swswldgldvtfvkwenktk dgdgkcsili asnetwrkvh csrgyaravc kiplspdytg
iailfavlcllglislaiwf llqrshirwt gfssvryehg tnedevmlps fhd

Antibodies suitable for practicing the methods described herein are preferably monoclonal, and can include, but are not limited to, human, humanized or chimeric antibodies, including single chain antibodies, Fab fragments, F(ab′) fragments, fragments produced by a Fab expression library, and/or antigen-binding fragments of any of the above. Antibody reagents also include immunoglobulin molecules and immunologically active portions of immunoglobulin molecules, i.e., molecules that contain at least one, at least two, at least three or more antigen binding sites that specifically bind a dendritic cell marker, such as DEC205. Such immunoglobulin molecules can be of any type (e.g., IgG, IgE, IgM, IgD, IgA and IgY), class (e.g., IgG1, IgG2, IgG3, IgG4, IgAQ1 and IgA2) or subclass of immunoglobulin molecule, as understood by one of skill in the art.

Additional types of antibodies include, but are not limited to, chimeric, humanized, and human antibodies. For application in man, it is often desirable to reduce immunogenicity of antibodies originally derived from other species, like mouse. This can be done by construction of chimeric antibodies, or by a process called “humanization”. In this context, a “chimeric antibody” is understood to be an antibody comprising a domain (e.g. a variable domain) derived from one species (e.g. mouse) fused to a domain (e.g. the constant domains) derived from a different species (e.g. human).

The term “monoclonal antibody” as used herein refers to a population of antibodies that comprise an identical antigen-binding domain. In some embodiments, a monoclonal antibody can be produced by a single B cell clone, B cell hybrodima or its equivalent. Such a cell produces only one antibody, such that all antibodies produced by such a clone have the same antigen-binding domain. In contrast to polyclonal antibody preparations that typically include different antibodies directed against different determinants (epitopes) on a given target antigen, each monoclonal antibody is directed against a single determinant on the antigen. The term “monoclonal antibody” as used herein is not limited to antibodies produced through hybridoma technology. It is to be understood that the term “monoclonal antibody” refers to an antibody that is derived from a single isolated clone, including any eukaryotic, prokaryotic or phage clone, and not the method by which the antibody is produced. For example, the monoclonal antibodies to be used in accordance with the methods and compositions described herein can be made by the hybridoma method first described by Kohler et al., Nature 256:495 (1975), or may be made by recombinant DNA methods (see, e.g., U.S. Pat. No. 4,816,567). The “monoclonal antibodies” can also be isolated from phage antibody libraries using the techniques described in Clackson et al., Nature 352:624-628 (1991) or Marks et al., J. Mol. Biol. 222:581-597 (1991), for example. A wide variety of methods for producing constructs with the antigen-binding domain of monoclonal antibodies are known to those of ordinary skill in the art.

Exemplary monoclonal antibodies that target DEC-205 are commercially available and include, but are not limited to Recombinant Anti-LY75/DEC-205 antibody [EPR5233](Cat. No. ab124897, Abcam, Cambridge, UK); DEC-205 F-4 (Cat. No. sc-515016, Santa Cruz Biotechnologies, Santa Cruz, CA); Mouse Anti-LY75 Recombinant Antibody (clone DEC-205) (Cat. No. FAMAB-1751CQ, Creative Biolabs, Shirley, NY); DEC-205 (MG38) (Cat. No. sc-23952, Santa Cruz Biotechnologies, Santa Cruz, CA); and human DEC-205/CD205 antibody (Cat. No. MAB2047; R&D Systems; Minneapolis, MN).

The sequences for the anti-DEC-205 antibody can be

anti-DEC205 NLDC145HC mulgG1_4m (SEQ ID NO: 19)
(heavy chain):
EVKLLESGGGLVQPGGSLRLSCAASGFTFNDFYMNWIRQPPGQAPEWLGV
IRNKGNGYTTEVNTSVKGRFTISRDNTQNILYLQMNSLRAEDTAIYYCAR
GGPYYYSGDDAPYWGQGVMVTVSSAKTTPPSVYPLAPGSAAQTNSMVTLG
CLVKGYFPEPVTVTWNSGSLSSGVHTFPAVLQSDLYTLSSSVTVPSSTWP
SETVTCNVAHPASSTKVDKKIVPRDCGCKPCICTVPPVSSVFIFPPKPKD
VLTITLTPKVTCVVVDISKDDPEVQFSWFVDDVEVHTAQTQPREEQFNST
FRSVSELPIMHQDWLNGKAFACAVNSAAFPAPIEKTISKTKGRPKAPQVY
TIPPPKEQMAKDKVSLTCMITDFFPEDITVEWQWNGQPAENYKNTQPIMD
TDGSYFVYSKLNVQKSNWEAGNTFTCSVLHEGLHNHHTEKSLSHSPGK
anti-DEC205_NLDC145LC_muKappa (SEQ ID NO: 20)
(light chain):
DIQMTQFPSFLSTSLGNSITITCHASQNIKGWLAWYQQKSGNAPQLLIYK
AFSLQSGVPSRFSGSGSGTDYIFTISNLQPEDIATYYCQHYQSFPWTFGG
GTKLELKRADAAPTVSIFPPSSEQLTSGGASVVCFLNNFYPKDINVKWKI
DGSERQNGVLNSWTDQDSKDSTYSMSSTLTLTKDEYERHNSYTCEATHKT
STSPIVKSFNRNEC

Antibodies useful in the present methods can be described or specified in terms of the particular CDRs they comprise. The compositions and methods described herein encompass the use of an antibody or derivative thereof comprising a heavy or light chain variable domain, where the variable domain comprises (a) a set of three CDRs, and (b) a set of four framework regions, and in which the antibody or antibody derivative thereof specifically binds a dendritic cell marker.

Antibodies can be produced in bacteria, yeast, fungi, protozoa, insect cells, plants, or mammalian cells (see e.g., Frenzel et al. (2013) Front Immunol. 4: 217). A mammalian expression system is generally preferred for manufacturing most therapeutic proteins, such as antibodies, as they require post-translational modifications. A variety of mammalian cell expression systems are now available for expression of antibodies, including but not limited to immortalized Chinese hamster ovary (CHO) cells, mouse myeloma (NSO), mouse L-cells, myeloma cell lines like J558L and Sp2/0, baby hamster kidney (BHK), or human embryo kidney (HEK-293).

As used herein, the term “Complementarity Determining Regions” (CDRs, i.e., CDR1, CDR2, and CDR3) refers to the amino acid residues of an antibody variable domain the presence of which are necessary for specific antigen binding. Each variable domain typically has three CDR regions identified as CDR1, CDR2 and CDR3. Each complementarity determining region can comprise amino acid residues from a “complementarity determining region” as defined by Kabat (i.e., about residues 24-34 (L1), 50-56 (L2) and 89-97 (L3) in the light chain variable domain and 31-35 (H1), 50-65 (H2) and 95-102 (H3) in the heavy chain variable domain). Likewise, “frameworks” (FWs) comprise amino acids 1-23 (FW1), 35-49 (FW2), 57-88 (FW3), and 98-107 (FW4) in the light chain variable domain and 1-30 (FW1), 36-49 (FW2), 66-94 (FW3), and 103-113 (FW4) in the heavy chain variable domain taking into account the Kabat numbering system (Kabat et al., Sequences of Proteins of Immunological Interest, 5th Ed. Public Health Service, National Institutes of Health, Bethesda, Md. (1987, 1991)).

The Kabat residue designations do not always correspond directly with the linear numbering of the amino acid residues. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard” Kabat numbered sequence. Methods and computer programs for determining sequence similarity are publicly available, including, but not limited to, the GCG program package (Devereux et al., Nucleic Acids Research 12: 387, 1984), BLASTP, BLASTN, FASTA (Altschul et al., J. Mol. Biol. 215:403 (1990), and the ALIGN program (version 2.0). The well-known Smith Waterman algorithm may also be used to determine similarity. The BLAST program is publicly available from NCBI and other sources (BLAST Manual, Altschul, et al., NCBI NLM NIH, Bethesda, Md. 20894; BLAST 2.0 at http://www.ncbi.nlm.nih.gov/blast/). In comparing sequences, these methods account for various substitutions, deletions, and other modifications.

### Cancer

The methods and compositions described herein can be used for treating cancer in a subject, in part, by enhancing anti-tumor immunity and/or improving antigen-presentation of cancer antigens to the immune system. In some embodiments of these aspects and all such aspects described herein, the subject in need thereof has or has been diagnosed with cancer.

In certain embodiments, the cancer is metastatic or has the potential to be metastatic. Metastasis can be local or distant. Metastasis is a sequential process, contingent on tumor cells breaking off from the primary tumor, traveling through the bloodstream, and stopping at a distant site. At the new site, the cells establish a blood supply and can grow to form a life-threatening mass. Both stimulatory and inhibitory molecular pathways within the tumor cell regulate this behavior, and interactions between the tumor cell and host cells in the distant site are also significant.

Metastases are most often detected through the sole or combined use of magnetic resonance imaging (MRI) scans, computed tomography (CT) scans, blood and platelet counts, liver function studies, chest X-rays and bone scans in addition to the monitoring of specific symptoms.

Examples of cancer that can be treated with the methods and compositions provided herein include, but are not limited to, carcinoma, lymphoma, blastoma, sarcoma, and leukemia. More particular examples of such cancers include, but are not limited to basal cell carcinoma, biliary tract cancer; bladder cancer; bone cancer; brain and CNS cancer; breast cancer; cancer of the peritoneum; cervical cancer; cholangiocarcinoma; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); lymphoma including Hodgkin's and non-Hodgkin's lymphoma; melanoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; teratocarcinoma; testicular cancer; thyroid cancer; uterine or endometrial cancer; cancer of the urinary system; vulval cancer; as well as other carcinomas and sarcomas; as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), tumors of primitive origins and Meigs' syndrome.

In some embodiments, a fusion protein comprising a saposin or prosaposin targeted to dendritic cells as described herein is used in combination with at least one additional anti-cancer therapy, such as an anti-cancer agent or chemotherapeutic, X-rays, gamma rays or other sources of radiation to destroy cancer stem cells and/or cancer cells.

Examples of anti-cancer therapies include, but are not limited to, e.g., surgery, chemotherapeutic agents, growth inhibitory agents, immune checkpoint inhibitors, cytotoxic agents, agents used in radiation therapy, anti-angiogenesis agents, apoptotic agents, anti-tubulin agents, and other agents to treat cancer, such as anti-HER2 antibodies (e.g., HERCEPTIN®), anti-CD20 antibodies, an epidermal growth factor receptor (EGFR) antagonist (e.g., a tyrosine kinase inhibitor), HER1/EGFR inhibitor (e.g., erlotinib (TARCEVA®)), platelet derived growth factor inhibitors (e.g., GLEEVEC™ (Imatinib Mesylate)), a COX2 inhibitor (e.g., celecoxib), interferons, cytokines, and other bioactive and organic chemical agents, etc. An anti-cancer therapy can include a cytotoxic agent, such as a radioactive isotope (e.g. At211, I131, I125, Y90, Re186, Re188, Sm153, Bi212, P32 or a radioactive isotope of Lu), chemotherapeutic agents, and toxins such as small molecule toxins or enzymatically active toxins of bacterial, fungal, plant or animal origin, including active fragments and/or variants thereof.

Non-limiting examples of chemotherapeutic agents can include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammalI and calicheamicin omegalI (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE, vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (Tykerb.); inhibitors of PKC-alpha, Raf, H-Ras, EGFR (e.g., erlotinib (Tarceva®)) and VEGF-A that reduce cell proliferation and pharmaceutically acceptable salts, acids or derivatives of any of the above. In addition, the methods of treatment can further include the use of radiation or radiation therapy.

By “reduce” or “inhibit” in terms of the cancer treatment methods described herein refers to a reduction in at least one parameter or symptom of a cancer by at least 10%, at least 20% at least 30%, at least 40%, at least 45%, at least 50%, at least 55%, at least 60%, at least 65%, at least 70%, at least 75% at least 80%, at least 85%, at least 90%, or at least 95% or greater. Parameters or symptoms of cancer that can be reduced or inhibited with the methods described herein include, but are not limited to, the presence or size of metastases or micrometastases, the size of the primary tumor, the presence or the size of the dormant tumor, tumor growth rate, pain, degree of angiogenesis in the tumor, degree of antigen presentation by dendritic cells, number of T lymphocytes in the tumor microenvironment etc. A patient or subject who is being treated for a cancer or tumor is one who a medical practitioner has diagnosed as having such a condition. Diagnosis can be by any suitable means.

### Dosage and Administration

The methods and compositions provided herein enhance anti-tumor immunity in a subject by administering a therapeutically effective amount of a saposin or prosaposin targeted to dendritic cells. In one embodiment, the subject can be a mammal. In another embodiment, the mammal can be a human, although the approach is effective with respect to all mammals.

The appropriate dosage range for a composition as described herein or a given combination anti-cancer agent depends upon the potency of the agent, and includes amounts large enough to produce the desired effect, e.g., treatment of one or more symptoms associated with cancer (e.g., tumor size, tumor growth, pain, etc). Although adverse side effects are often associated with anti-cancer agents, the dosage should not be so large as to cause unacceptable or life-threatening adverse side effects. Generally, the dosage will vary with the type of inhibitor, and with the age, condition, and sex of the patient. The dosage can be determined by one of skill in the art and can also be adjusted by the individual physician in the event of any complication.

The effective amount may be based upon, among other things, the size of the agent, the biodegradability of the agent, the bioactivity of the agent, the bioavailability of the agent or the formulation of the agent. For example, if the compound does not degrade quickly, is bioavailable and highly active, a smaller amount will be required to be effective. One of skill in the art can perform routine empirical activity tests for a compound to determine the bioactivity in bioassays and thus determine the effective amount.

As for when the compound, composition and/or agent is to be administered, one skilled in the art can determine when to administer such a compound and/or agent. The administration may be constant for a certain period of time or periodic and at specific intervals. The agent can be delivered hourly, daily, weekly, monthly, yearly (e.g. in a time release form) or as a one-time delivery. The delivery may be continuous delivery for a period of time, e.g. intravenous delivery. In one embodiment of the methods described herein, the agent is administered at least once per day. In one embodiment of the methods described herein, the agent is administered daily. In one embodiment of the methods described herein, the agent is administered every other day. In one embodiment of the methods described herein, the agent is administered every 6 to 8 days. In one embodiment of the methods described herein, the agent is administered weekly.

As one of skill in the art will appreciate, the dosage of a composition as described herein can vary depending upon the dosage form employed and the route of administration utilized. Compositions, methods, and uses that exhibit large therapeutic indices (i.e., the dose ratio between toxic and therapeutic effects) are preferred. A therapeutically effective dose can be estimated initially from cell culture assays. Also, a dose can be formulated in animal models to achieve a circulating plasma concentration range that includes the IC50, which achieves a half-maximal inhibition of measured function or activity as determined in cell culture, or in an appropriate animal model. The effects of any particular dosage can be monitored by a suitable bioassay. A therapeutically effective amount is an amount of an agent that is sufficient to produce a statistically significant, measurable change of a given symptom of a cancer (see “Efficacy” below). Such effective amounts can also be gauged in clinical trials as well as animal studies for a given agent. In some embodiments, the therapeutic efficacy can be estimated by the ED50 in an animal model (the dose therapeutically effective in 50% of the population) or in a cell cytotoxicity assay (where at least 50% of the cancer cells are killed).

Therapeutic compositions can be conventionally administered in a unit dose. The term “unit dose” when used in reference to a therapeutic composition refers to physically discrete units suitable as unitary dosage for the subject, each unit containing a predetermined quantity of an anti-cancer agent calculated to produce the desired therapeutic effect in association with the required physiologically acceptable diluent, i.e., carrier, or vehicle.

The agents described herein can be administered to a subject in need thereof by any appropriate route which results in an effective treatment in the subject. For example, agents useful in the methods and compositions described herein can be administered topically, intravenously (by bolus or continuous infusion), orally, by inhalation, intraperitoneally, intramuscularly, subcutaneously, intracavity, and can be delivered by peristaltic means, if desired, or by other means known by those skilled in the art. The agent can be administered systemically, if so desired. An agent can be targeted by means of a targeting moiety, such as e.g., an antibody or targeted liposome technology.

### Combination Therapies:

Combination therapy using a saposin or prosaposin targeted to a dendritic cell, with an additional anti-cancer treatment, can comprise administration of the therapeutics to a subject concurrently. The term “concurrently” is not limited to the administration of the cancer therapeutics at exactly the same time, but rather, it is meant that they are administered to a subject in a sequence and within a time interval such that they can act together (e.g., synergistically to provide an increased benefit than if they were administered otherwise). For example, the combination therapies can be administered at the same time or sequentially in any order at different points in time; however, if not administered at the same time, they should be administered sufficiently close in time so as to provide the desired therapeutic effect, preferably in a synergistic fashion. The combination cancer therapeutics can be administered separately, in any appropriate form and by any suitable route. When the combination therapies are not administered in the same pharmaceutical composition, it is understood that they can be administered in any order to a subject in need thereof. For example, a first prophylactically and/or therapeutically effective regimen can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of the second cancer therapeutic, to a subject in need thereof.

Immune checkpoint proteins interact with specific ligands that send a signal into the T cell and switch off or inhibit T cell function. By expressing high levels of checkpoint proteins on their surface, cancer cells can control the function of T cells that enter the tumor microenvironment, thus suppressing the anticancer immune response. The immune checkpoint protein Programmed Death-1 (PD-1) is a key immune checkpoint receptor expressed by activated T and B cells and mediates immunosuppression. PD-1 is a member of the CD28 family of receptors, which includes CD28, CTLA-4, ICOS, PD-1, and BTLA. Two cell surface glycoprotein ligands for PD-1 have been identified, Programmed Death Ligand-1 (PD-L1) and Programmed Death Ligand-2 (PD-L2), that are expressed on antigen-presenting cells as well as many human cancers and have been shown to downregulate T cell activation and cytokine secretion upon binding to PD-1 (Freeman et al., 2000; Latchman et al., 2001). Inhibition of the PD-1/PD-L1 interaction can promote potent antitumor activity. Examples of PD-1 inhibitors include, but are not limited to, Pembrolizumab (MK-3475), Nivolumab (MDX-1106), Cemiplimab-rwlc (REGN2810), Pidilizumab (CT-011), Spartalizumab (PDR001), tislelizumab (BGB-A317), PF-06801591, AK105, BCD-100, BI 754091, JS001, LZM009, MEDI0680, MGA012, Sym021, TSR-042. Examples of PD-L1 inhibitors include, but are not limited to, Atezolizumab (MPDL3280A), Durvalumab (MED14736), Avelumab (MSB0010718C), BGB-A333, CK-301, CS1001, FAZ053, KN035, MDX-1105, MSB2311, SHR-1316. Additional immune checkpoint inhibitors include, but are not limited to inhibitors of PD-L2, CTLA-4, B7-H3, B7-H4, BTLA, HVEM, GALS, LAG3, TIM-3, VISTA, KIR, 2B4 (belongs to the CD2 family of molecules and is expressed on all NK, y6, and memory CD8+ (4) T cells), CD160 (also referred to as BY55), CGEN-15049, CHK1 and CHK2 kinases, A2aR and various B-7 family ligands (including, but are not limited to, B7-1, B7-2, B7-DC, B7-H1, B7-H2, B7-H3, B7-H4, B7-H5, B7-H6 and B7-H7).

When administered in combination with a composition as described herein, the anti-cancer agent or drug can be administered in an amount or dose that is lower (e.g., at least 20%, at least 30%, at least 40%, or at least 50% lower) or the same as the amount or dosage of the agent used individually, e.g., as a monotherapy.

Currently available anti-cancer therapies and their dosages, routes of administration and recommended usage are known in the art and have been described in such literature as the Physician's Desk Reference (60th ed., 2017).

### Compositions, Formulations and Packaging

Also provided herein are compositions, including pharmaceutical compositions, comprising a saposin or prosaposin targeted to a dendritic cell as described herein. In one embodiment, the compositions are pharmaceutical compositions. Pharmaceutical compositions for use with the methods described herein can be formulated in conventional manner using one or more physiologically acceptable carriers or excipients. Thus, the agent can be formulated for administration by, for example, aerosol, intravenous, oral or topical route. The compositions can be formulated for intralesional, intratumoral, intraperitoneal, subcutaneous, intramuscular or intravenous injection; infusion; liposome-mediated delivery; topical, intrathecal, gingival pocket, per rectum, intrabronchial, nasal, transmucosal, intestinal, oral, ocular or otic delivery.

Techniques and formulations generally may be found in Remmington's Pharmaceutical Sciences, Meade Publishing Co., Easton, PA. For injection, the compounds can be formulated in liquid solutions, preferably in physiologically compatible buffers such as Hank's solution or Ringer's solution. In addition, the agent may be formulated in solid form and redissolved or suspended immediately prior to use. Lyophilized forms are also included.

For oral administration, the pharmaceutical composition can take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods well known in the art. Liquid preparations for oral administration can take the form of, for example, solutions, syrups or suspensions, or they may be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., pharmaceutically acceptable oils, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate.

Preparations for oral administration can be suitably formulated to give controlled release of the active compound. For buccal administration the compositions can take the form of tablets or lozenges formulated in conventional manner. For administration by inhalation, the compounds for use as described herein are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g., gelatin for use in an inhaler or insufflator may be formulated containing a powder mix of the compound and a suitable powder base such as lactose or starch.

The saposin or prosaposin targeted to a dendritic cell can be formulated for parenteral administration by injection, e.g., by bolus injection or continuous infusion. Formulations for injection can be presented in unit dosage form, e.g., in ampoules or in multi-dose containers, with or without an added preservative. The compositions can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use.

The compositions can, if desired, be presented in a pack or dispenser device which can contain one or more unit dosage forms containing the active ingredient. The pack can for example comprise metal or plastic foil, such as a blister pack. The pack or dispenser device can be accompanied by instructions for administration.

### Efficacy

The efficacy of a given treatment for a cancer can be determined by the skilled clinician. However, a treatment is considered “effective treatment,” as the term is used herein, if any one or all of the signs or symptoms of the cancer is/are altered in a beneficial manner, or other clinically accepted symptoms or markers of disease are improved, or ameliorated, e.g., by at least 10% following treatment with a composition as described herein. Efficacy can also be measured by failure of an individual to worsen as assessed by stabilization of the disease, or the need for medical interventions (i.e., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing progression of the cancer; or (2) relieving the disease, e.g., causing regression of symptoms; and (3) preventing or reducing the likelihood of the development of the disease, or preventing secondary diseases/disorders associated with the cancer (e.g., cancer metastasis).

An effective amount for the treatment of a disease means that amount which, when administered to a mammal in need thereof, is sufficient to result in effective treatment as that term is defined herein, for that disease. Efficacy of an agent can be determined by assessing physical indicators of the disease or desired response, such as e.g., pain, tumor size, tumor growth rate, blood cell count, etc. It is well within the ability of one skilled in the art to monitor efficacy of administration and/or treatment by measuring any one of such parameters, or any combination of parameters. Efficacy can be assessed in animal models of a condition described herein, for example animal models of cancer, e.g. a munne xenograft model. When using an experimental animal model, efficacy of treatment is evidenced when a statistically significant change in a marker is observed.

The technology may be described in any one of the following numbered paragraphs:

Paragraph 1: A composition comprising: a saposin or prosaposin targeted for delivery to a dendritic cell.

Paragraph 2: The composition of paragraph 1, wherein the saposin or prosaposin comprises an antigen binding moiety that binds a dendritic cell antigen.

Paragraph 3: The composition of paragraph 2, wherein the saposin or prosaposin is chemically conjugated to the antigen binding moiety, optionally via a chemical or peptide linker.

Paragraph 4: The composition of paragraph 2, wherein the dendritic cell antigen comprises DEC205.

Paragraph 5: The composition of paragraph 2, wherein the antigen binding moiety comprises an antibody or antigen binding fragment thereof.

Paragraph 6: The composition of paragraph 1, wherein the saposin comprises saposin A, saposin B, saposin C, and/or saposin D.

Paragraph 7: The composition of paragraph 1, further comprising a pharmaceutically acceptable carrier.

Paragraph 8: A fusion protein comprising a saposin or prosaposin conjugated to a moiety that targets the fusion protein to dendritic cells.

Paragraph 9: A method of treating cancer comprising administering the composition of any one of paragraphs 1-8 to a subject in need thereof.

Paragraph 10: A method of enhancing anti-tumor immunity in a subject, the method comprising administering a composition of any one of paragraphs 1-9 to a subject having cancer, wherein the anti-tumor immunity is increased by at least 10% as compared to the anti-tumor immunity in a subject not administered the composition of any one of paragraphs 1-9.

Paragraph 11: The method of paragraph 10, wherein the anti-tumor immunity is assessed by measuring T lymphocyte stimulation.

Paragraph 12: The method of paragraph 10, wherein the tumor volume is decreased in the subject by at least 20%.

Paragraph 13: The method of paragraph 10, wherein the cancer is melanoma.

Paragraph 14: The method of paragraph 10, wherein antigen presentation by dendritic cells is increased.

Paragraph 15: The method of paragraph 10, wherein the saposin or prosaposin degrades apoptotic vesicles from the tumor.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

### EXAMPLES

The harnessing of immunological mechanisms for cancer therapy is revolutionizing oncology treatment. As immune checkpoint blockade demonstrates, effective CD8 T cell responses are vital to protect against tumors. Central to robust T cell activation is their interaction with antigen-presenting cells, such as dendritic cells (DCs), which trigger efficient T cell priming and effector functions required for cancer protection. Unfortunately, antigen presentation and T cell activation in the tumor microenvironment (TME) are often suppressed, a phenomenon that facilitates immune escape of cancer and precludes effective therapy and cure. Therefore, an important goal of new treatment modalities is to restore productive antigen presentation in tumor DCs.

Recently, the inventors discovered a new mechanism how DCs efficiently process antigens derived from tumor cells based on the function of a group of lysosomal proteins called saposins. The single saposins A-D are derived from the precursor prosaposin (pSAP) and are able to disintegrate apoptotic vesicles from tumors to liberate enclosed antigens for subsequent presentation and T cell activation. Notably, pSAP-deficient mice failed to mount anti-tumor immunity and to control cancer growth. Moreover, it was found that cancer induces the hyperglycosylation of pSAP in tumor DCs, which leads to its secretion and consequent lack in lysosomes. This immune escape mechanism is triggered by the cytokine TGF-β and hampers efficient antigen processing and T cell induction. Notably, reconstitution of tumor DCs with functional pSAP protein rescued potent stimulation of T lymphocytes isolated from melanoma patients. Taken together, pSAP-based immunotherapy can be used for the treatment of cancer. Accordingly, the inventors used a method for generation of anti-DEC205 conjugated with recombinant pSAP, which is able to target functional pSAP to DCs to bypass endogenous hyperglycosylation and thus preserve its potent antigen-processing functions to trigger anti-tumor immunity.

For delivery of pSAP to dendritic cells, anti-DEC205 antibody is activated for chemical conjugation by sulfo-SMCC (sulfosuccinimidyl 4-[N-maleimidomethyl]cyclohexane-1-carboxylate) while the sulfhydryl-groups of the pSAP protein are exposed through incubation with 25 nM of TCEP-HCl (tris (2-carboxyethyl) phosphine hydrochloride). Excess TCEP-HCl and sulfo-SMCC are removed by using desalting columns, and the reduced pSAP is mixed with the activated antibody for coupling at a molar ratio of 1:1. The resulting anti-DEC-205/pSAP conjugate is concentrated and freed from unbound pSAP or antibodies by using 150 kD cutoff columns, and the successful conjugation of pSAP to anti-DEC-205 is verified by western blot analysis and enzyme-linked immunosorbent assay (ELISA).

Alternatively, it is specifically contemplated herein that a fusion protein (e.g., expressed from a vector or in a cell, which is also referred to as a “genetic fusion”) comprising a saposin or prosaposin conjugated or fused to an antibody or antigen binding fragment (including, but not limited to an scFv or other antigen-binding construct comprising the antigen-binding domains of an antibody), with or without a linker, can be used in place of a chemically conjugated prosaposin composition.

### Example 2

Antigen-specific T cell responses are central to protection against cancer. Cytotoxic CD8 T lymphocytes recognize tumor antigens presented by MHC-I molecules and subsequently deploy their effector functions, such as target cell killing and production of inflammatory cytokines. However, tumor cells often fail to directly activate T cells due to downregulation of their MHC-I pathway. Therefore, other antigen-presenting cells, such as dendritic cells (DCs), are critical to engulf tumor antigens for subsequent processing and display to MHC-I-restricted CD8 T cells in a process called cross-presentation. On a cellular level, this mechanism can be broadly divided into a cytosolic and vacuolar pathway. According to cytosolic processing, endosomal antigens are retrotranslocated into the cytosol for degradation by proteasomes and subsequent reimport into the endosome for MHC-I loading. By contrast, in the vacuolar pathway, the endosome is more autonomous and relies on its proteases for antigen processing. Among the different DC subsets, classical DC1 are especially efficient in cross-presentation and fulfill this function in tumor-draining lymph nodes for T cell priming as well as in the tumor microenvironment to activate tumor-infiltrating T lymphocytes. Abundance and proper function of immune cells, including antigen-presenting cells and lymphocytes, at the tumor site are vital for effective immunity and control of cancer growth. Unfortunately, tumors often develop mechanisms to evade immune responses, for example by producing immunosuppressive cytokines, such as TGF-3. Thus, a goal of cancer therapy is to target and overcome immune evasion to restore protective immunity.

Prosaposin is a precursor protein that is transported from the Golgi apparatus to the lysosome assisted by its chaperones sortilin. In the lysosome, cathepsins cleave prosaposin into the single saposins A-D. Saposins are also called sphingolipid activator proteins since they function as small, non-enzymatic cofactors for lysosomal hydrolases that are required for sphingolipid degradation. Moreover, at the low acidic pH of the endolysosomal compartment, saposins are able to interact with anionic phospholipids, such as phosphatidylserine (PS), exposed on intralysosomal vesicles. These membrane-perturbing properties facilitate vesicle disintegration and also pertain to apoptotic vesicles that characteristically contain PS in their lipid bilayers. In this context, tumors, owing to their uncontrolled growth kinetics, produce a substantial amount of dying cells and apoptotic bodies, which contain tumor antigens to potentially trigger the immune system. Notably, membrane-associated particulate antigen is more immunogenic than soluble protein and thus, antigen presentation pathways based on vesicular processing might be central to the induction of protective T cell immunity. In this study, we explored the impact of saposins on presentation of membrane-associated tumor antigen and activation of CD8 T cell responses that protect against cancer growth. This work also describes a mechanism how the tumor counteracts saposin-mediated processing by triggering prosaposin hyperglycosylation and secretion from tumor-associated DCs. Ultimately, the proof-of-principle of prosaposin is tested by targeting to DCs as a mode of cancer immunotherapy.

### Results

### Saposins Promote Cross-Presentation of Membrane-Associated Tumor Antigen

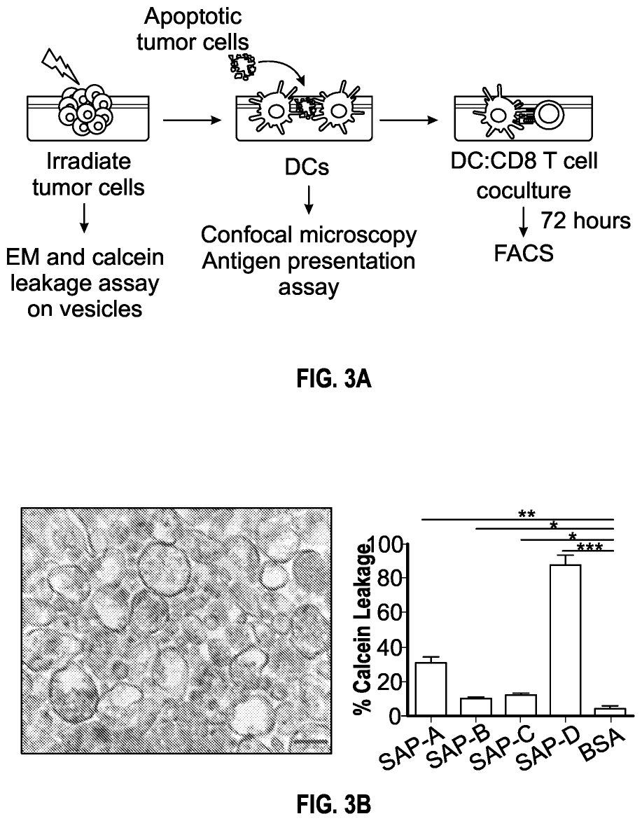

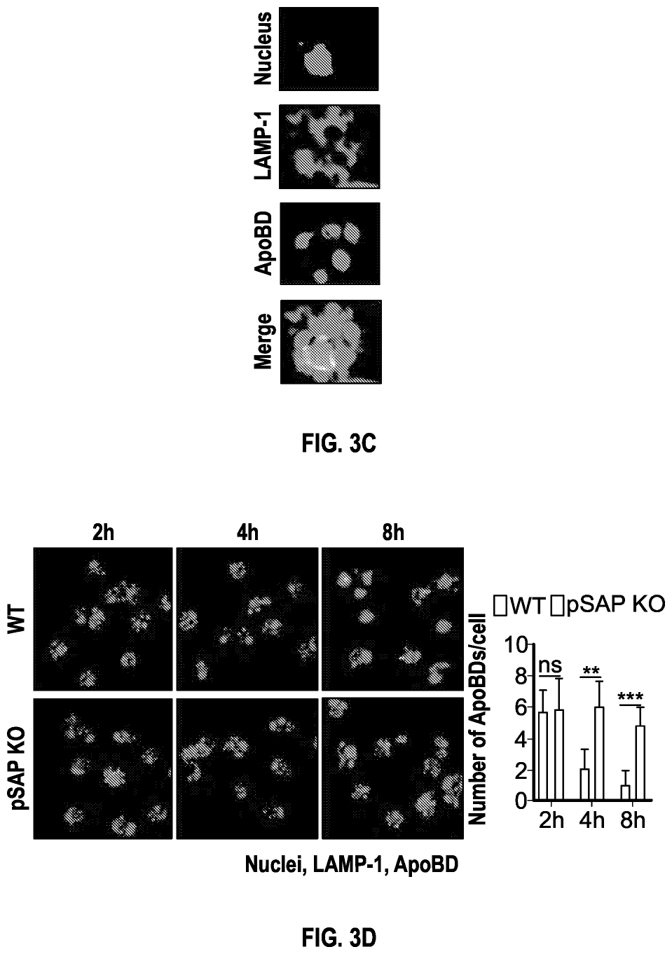

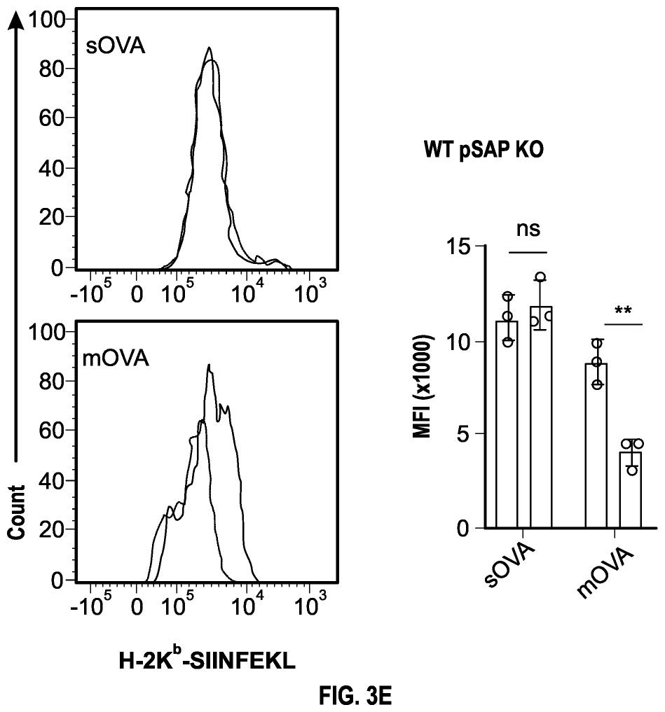

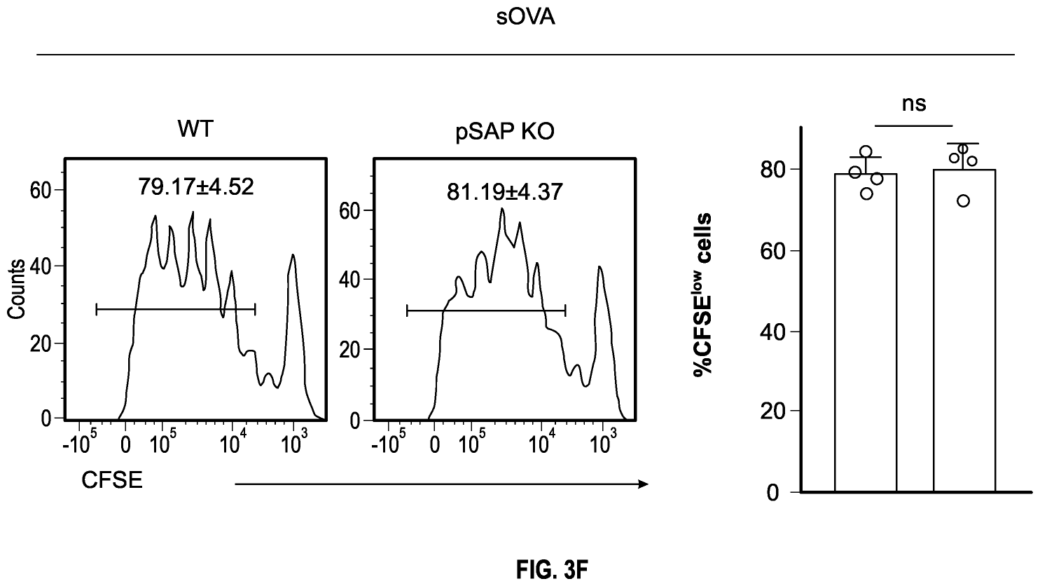

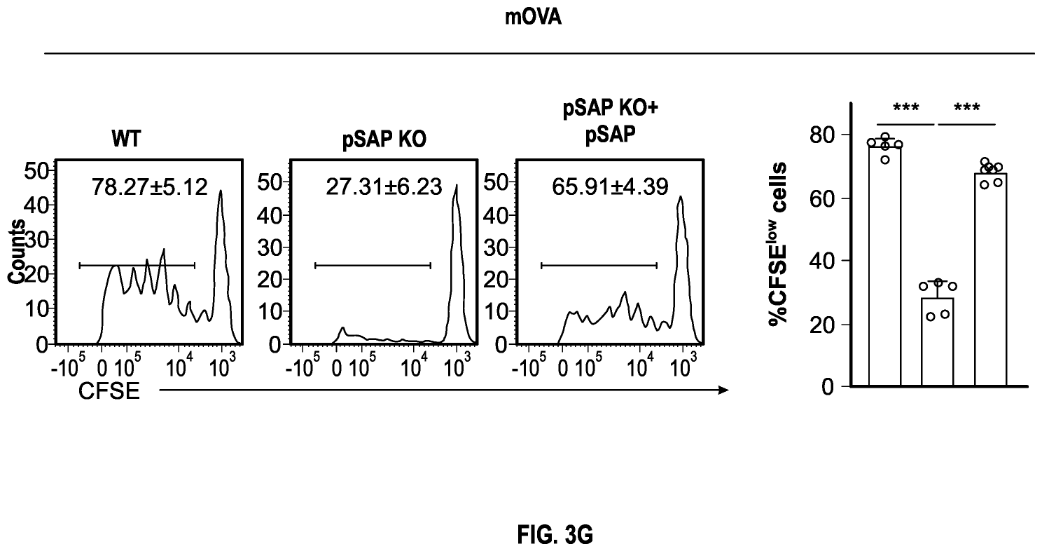

First, the effect of saposins on the integrity of apoptotic bodies derived from tumor cells is investigated. For this purpose, murine MCA fibrosarcoma cells were exposed to γ-irradiation (100 Gy) to trigger apoptotic cell death (FIG. 3A). Successful induction of apoptosis was controlled by measuring phosphatidylserine exposure using AnnexinV staining. Subsequently, apoptotic vesicles were purified from cell culture supernatants using differential ultracentrifugation (100,000 g pellets) and visualized by transmission electron microscopy (FIG. 3B). The fluorescent dye calcein was then loaded into those apoptotic vesicles using a liposome extruder with a 100 nm pore size. After incubation with different recombinant saposins, calcein release was measured and found that saposins disintegrate tumor cell-derived apoptotic vesicles when compared to control BSA (FIG. 3B).

The impact of saposins on processing of apoptotic bodies was explored in DCs. To this end, bone marrow-derived DCs were pulsed with CFSE-labeled apoptotic cells derived from y-irradiated MCA101 tumor cells and followed their fate along the endolysosomal compartment. Confocal microscopy revealed colocalization of apoptotic bodies with LAMP-1, indicating trafficking to saposin-containing lysosomes (FIG. 3C). When comparing the kinetic digestion of apoptotic cells in prosaposin-deficient or wild-type DCs, it was found that early uptake of apoptotic material was similar, suggesting that phagocytosis is not affected by saposin deficiency. However, at later time points after endocytosis, prosaposin-deficient DCs accumulated CFSE-labeled cells, demonstrating the importance of saposins for processing of apoptotic bodies in DCs (FIG. 3D).

To test for saposin-dependent cross-presentation and CD8 T cell activation, DCs were pulsed from prosaposin-KO or WT mice with apoptotic MCA101 cells expressing a membrane-associated form of the antigen ovalbumin (OVA), prior to coculture with OVA-specific CD8 T cells. First, productive antigen processing was analyzed by staining for the processed OVA epitope in complex with MHC-I (H-2k^{b}-SIINFEKL, SEQ ID NO: 25) on the DC surface. Flow cytometry demonstrated that processing of soluble OVA was equally efficient in pSAP-KO and WT DCs (FIG. 3E). However, processing and loading onto MHC-I of membrane-associated antigen derived from tumor cells was significantly hampered in the absence of prosaposin (FIG. 3E). These findings were in accordance with our T cell data, since pSAP-deficient DCs activated CD8 T cells responding to soluble OVA as efficient as WT DCs (FIG. 3F). In sharp contrast, when DCs were pulsed with tumor cells containing membrane-associated antigen, CD8 T cell activation by pSAP-KO DCs was strikingly reduced, as highlighted by their truncated CFSE dilution profile in flow cytometry (FIG. 3G). Notably, incubation of pSAP-deficient DCs with recombinant prosaposin fully reconstituted CD8 T cell activation (FIG. 3G). Taken together, saposins disintegrate apoptotic vesicles and process membrane-associated antigen for cross-presentation to CD8 T cells.

### Prosaposin is Required for Tumor Immunity

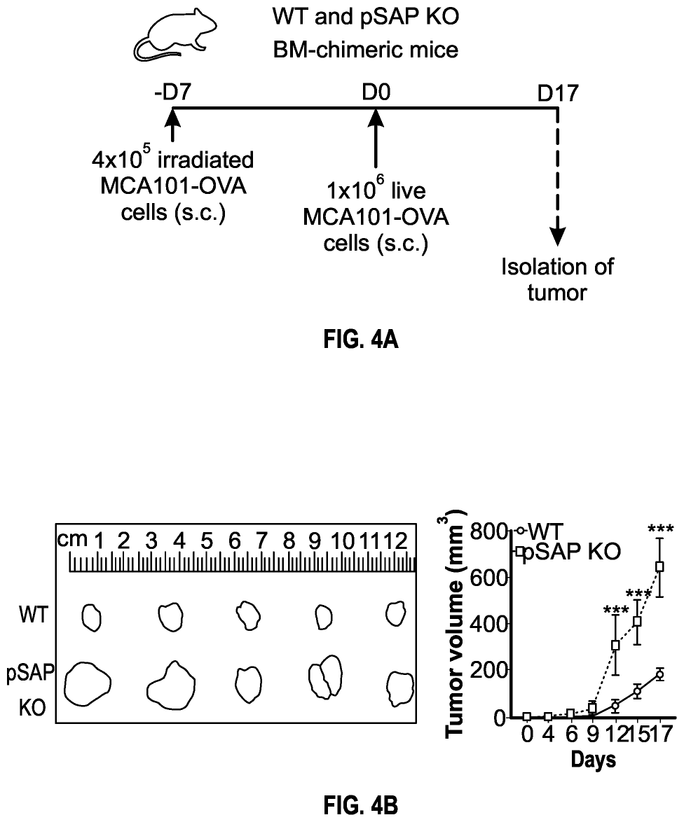

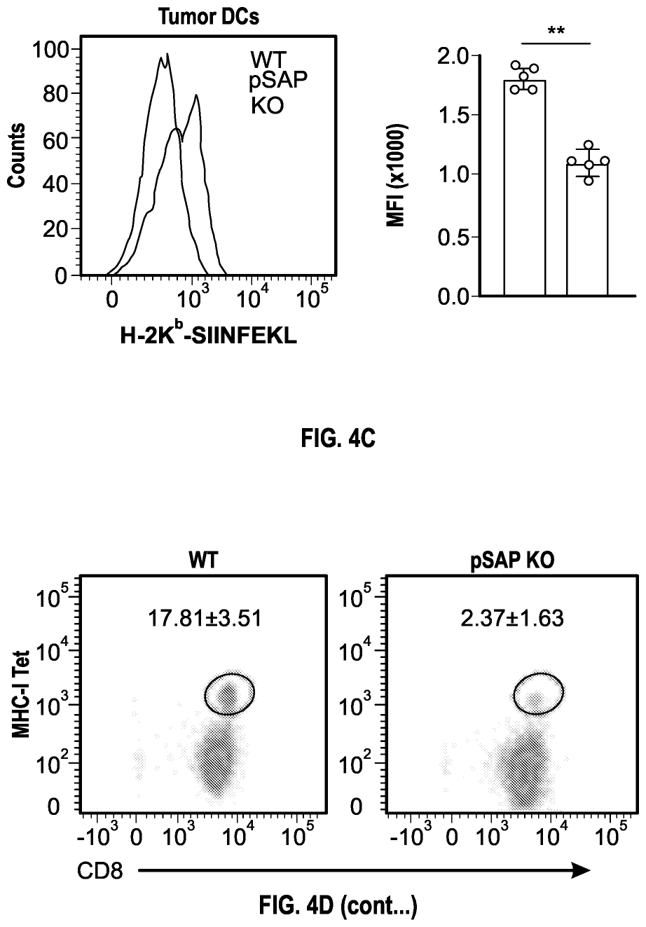

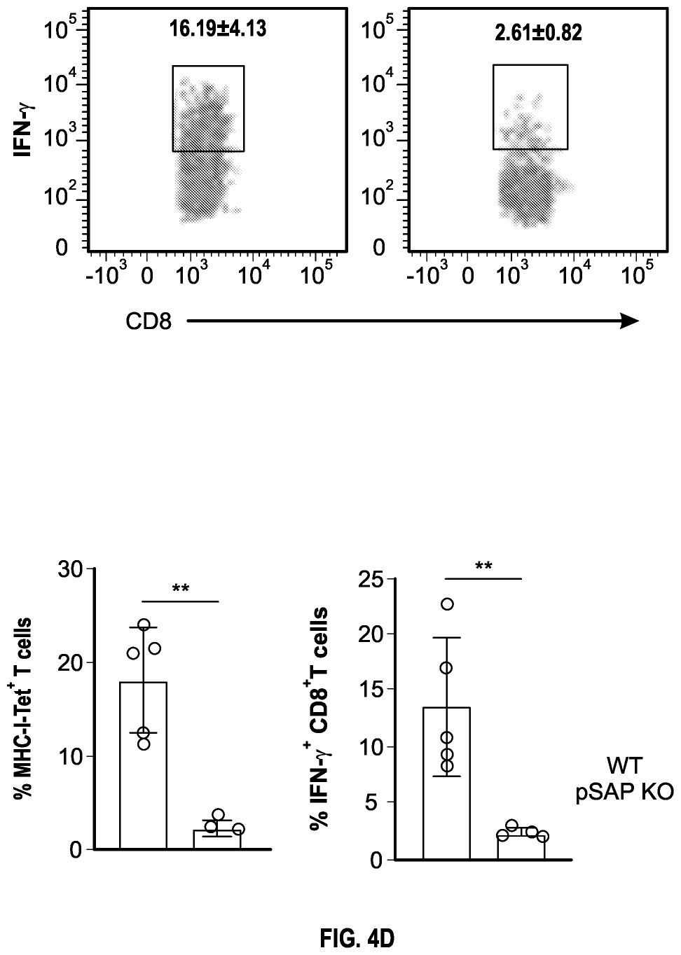

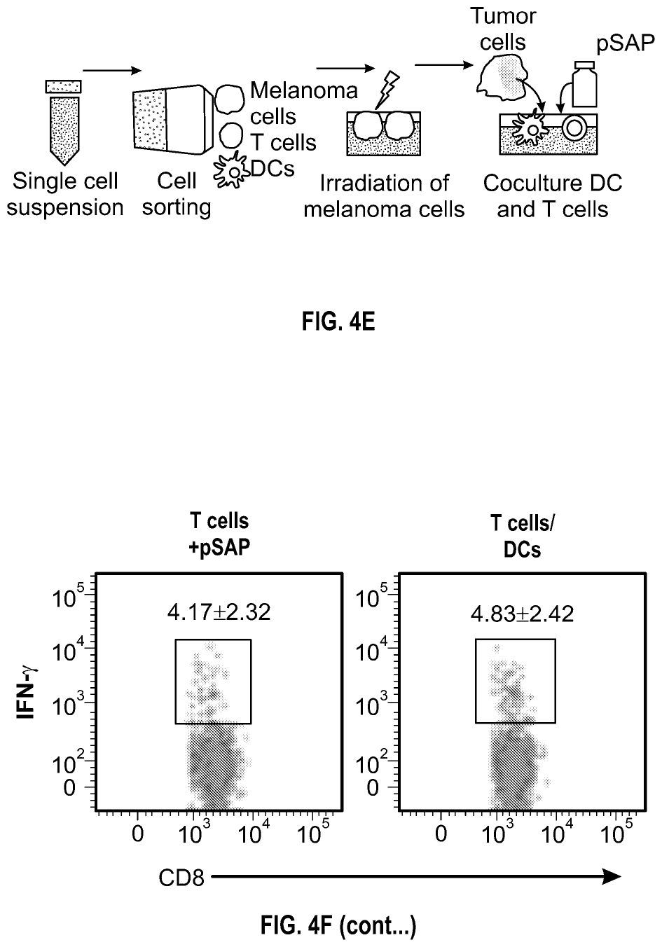

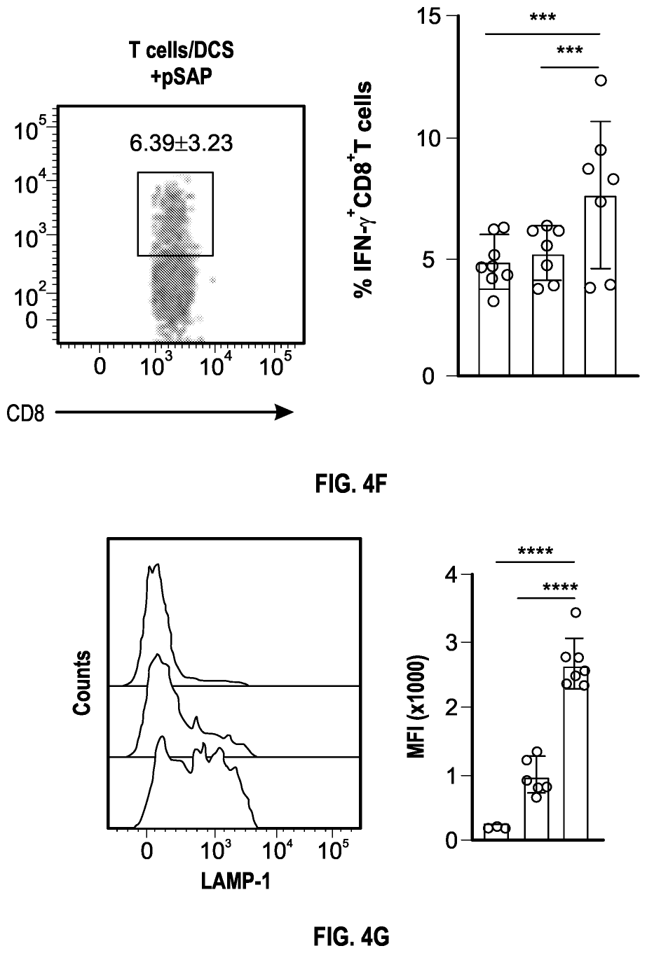

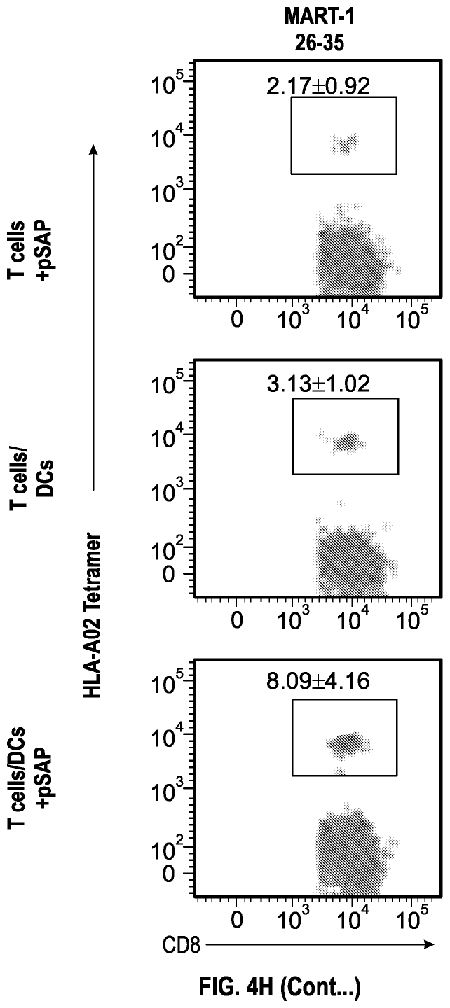

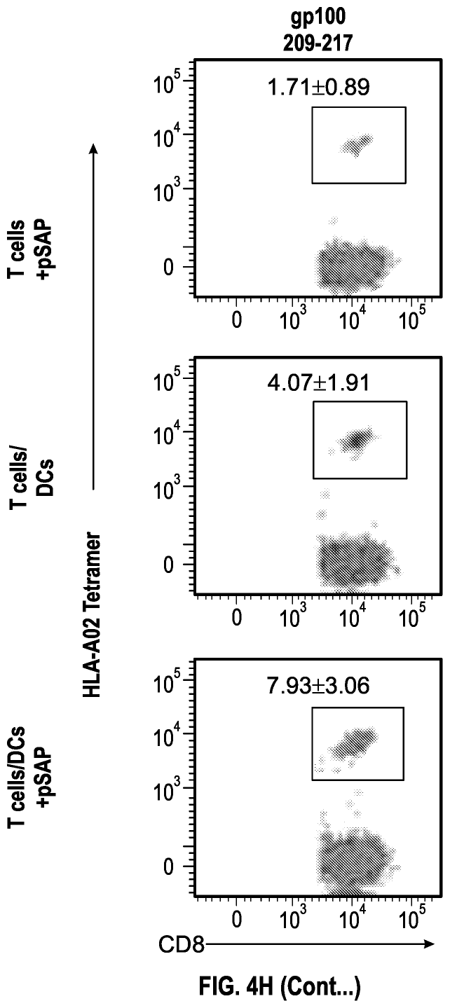

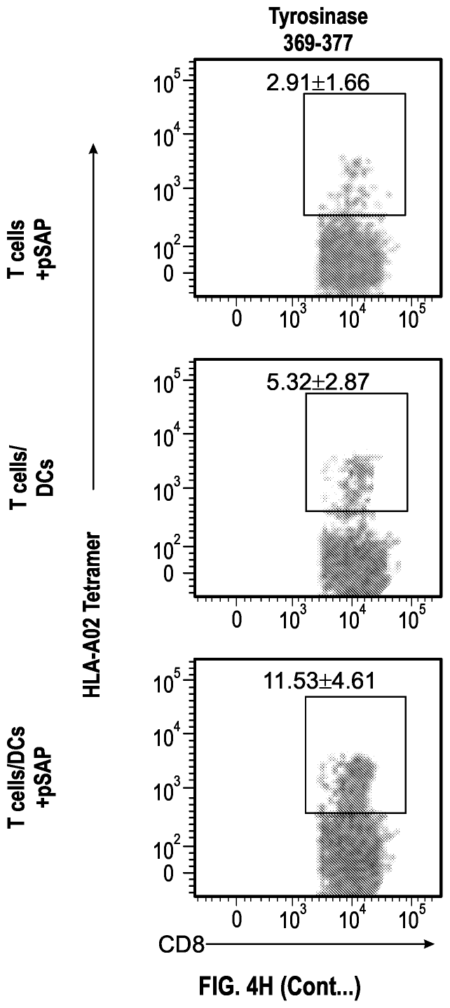

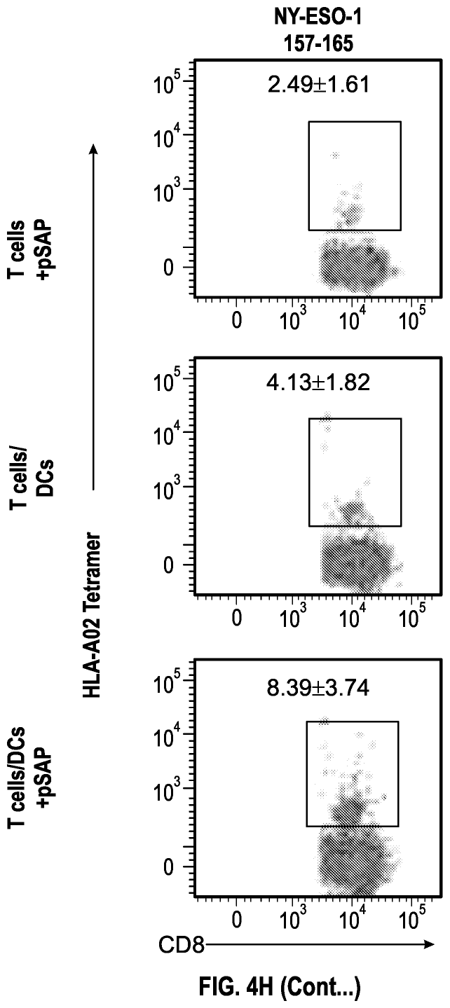

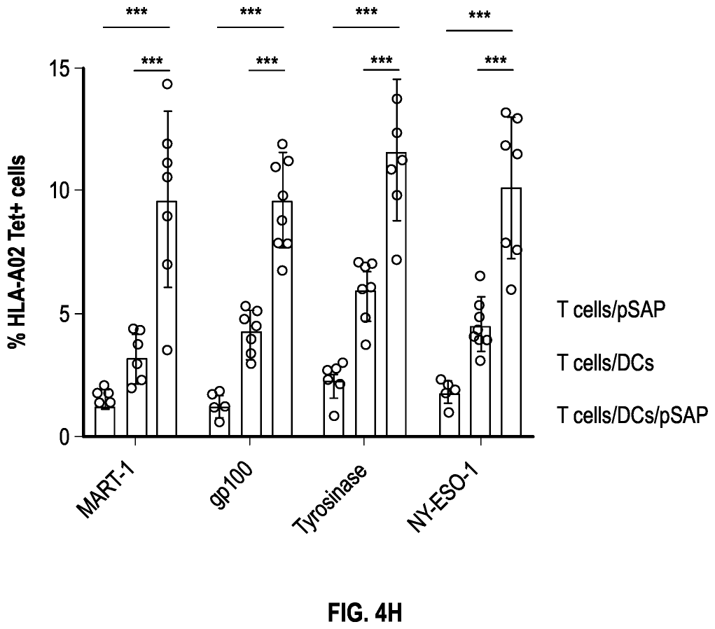

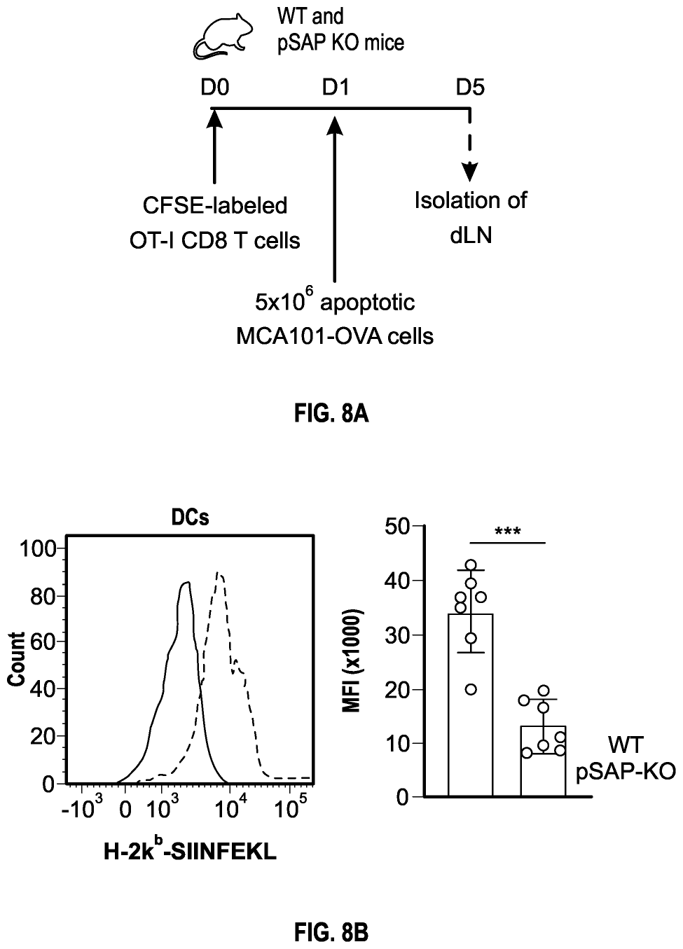

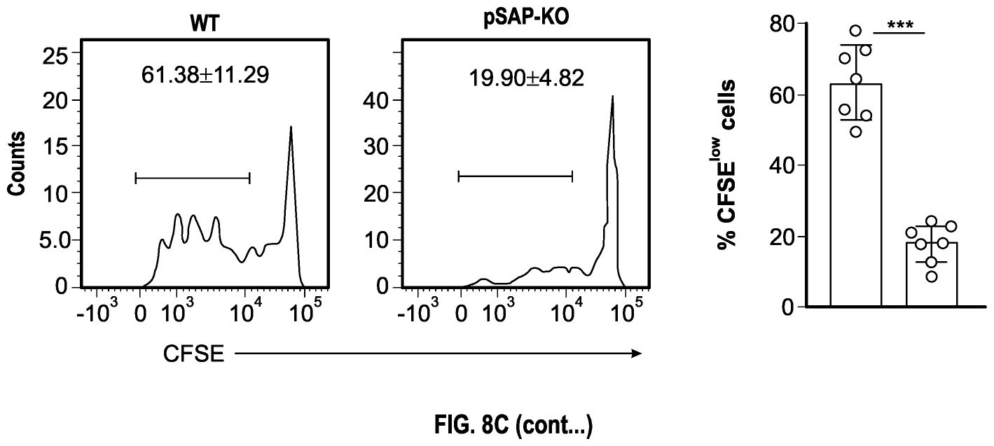

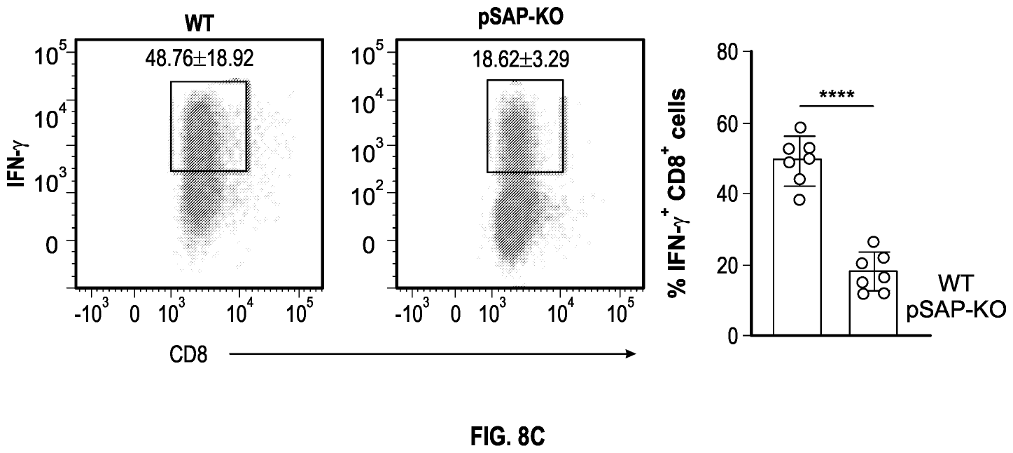

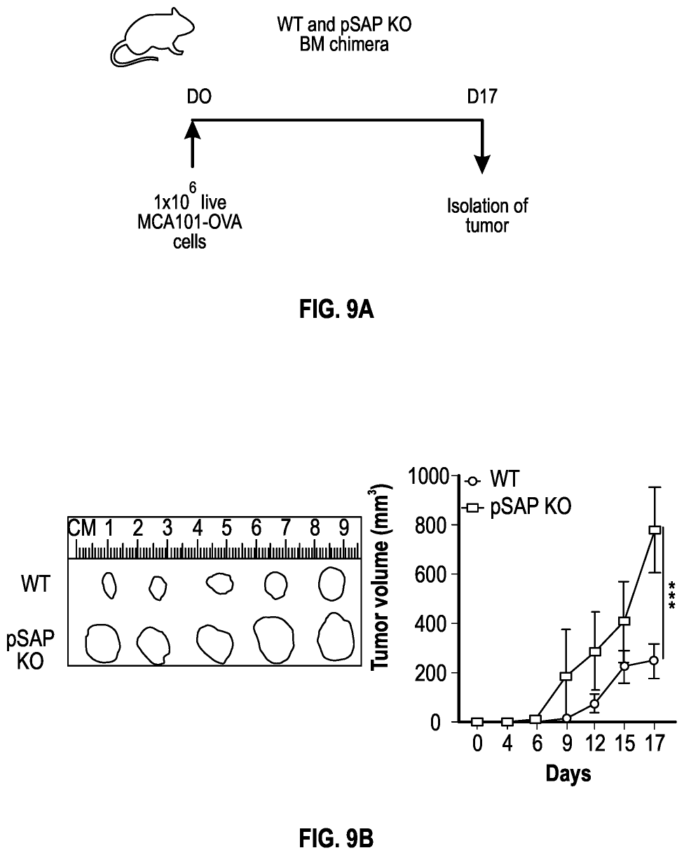

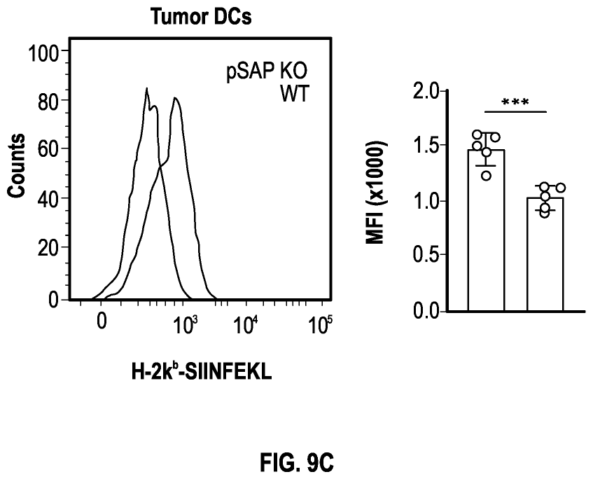

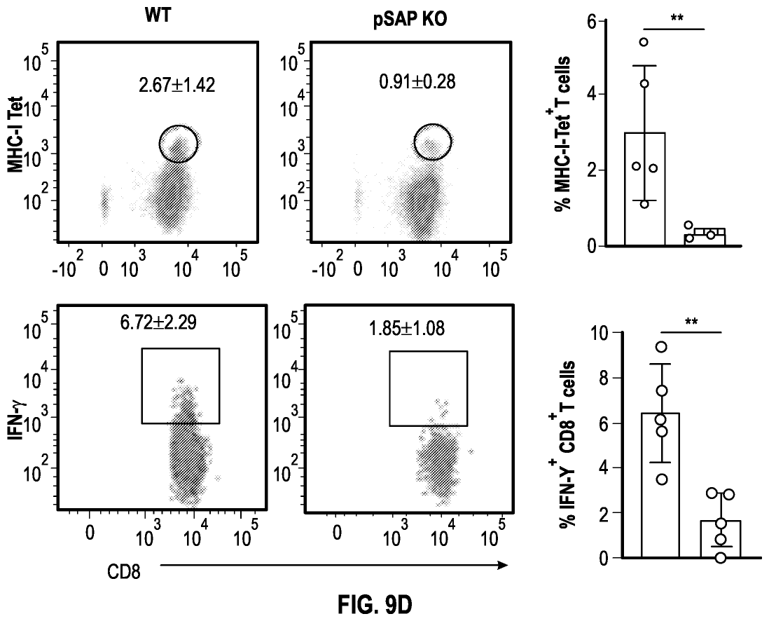

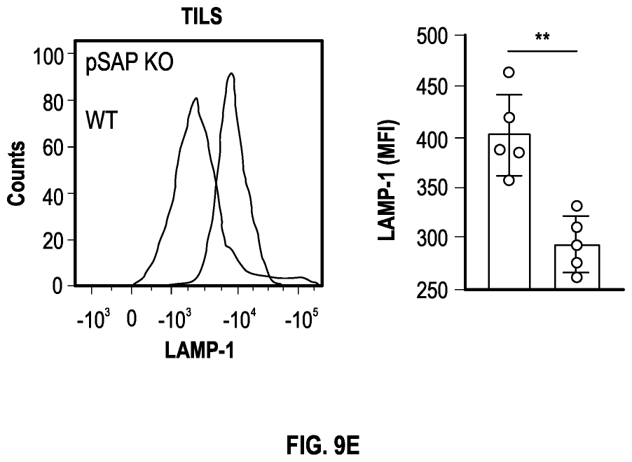

Next, it was investigated how prosaposin function affects T cell activation in vivo and protection against cancer. In order to assess T cell priming, naïve CFSE-labeled, OVA-specific CD8 T cells were transferred into pSAP-deficient or WT recipients, prior to subcutaneous administration of apoptotic MCA101-OVA cells (FIG. 8A). Four days after tumor cell injection, DCs were isolated from skin-draining lymph nodes and analyzed antigen processing. Expression of H-2k^{b}-SIINFEKL proved to be reduced in DCs from pSAP-KO when compared to WT mice (FIG. 8B). Moreover, antigen-specific proliferation and IFN-γ production by CD8 T cells from lymph nodes were severely hampered in the absence of prosaposin (FIG. 8C). Thus, prosaposin facilitates CD8 T cell priming in response to particulate antigen. Since straight pSAP-KO mice have a reduced life span, chimeric mice were then generated by transferring pSAP-KO or WT bone marrow to WT recipients in order to allow for tumor challenge experiments. In this context, mice were immunized with irradiated tumor cells and challenged them with a higher number of live MCA101-OVA cells one week later (FIG. 4A). Subsequently, tumor growth was monitored in the skin and found a drastic expansion of cancer in prosaposin deficiency (FIG. 4B). Flow cytometry analysis of isolated DCs from the tumor site showed pSAP-dependent decrease in antigen processing and presentation (FIG. 4C). Furthermore, MHC-I tetramer-mediated detection of antigen-specific CD8 T cells showed reduced frequency of tumor-infiltrating T cells as well as cytokine production in prosaposin-deficient mice (FIG. 4D). In addition, bone marrow-chimeric mice were challenged with live tumor cells without prior vaccination (FIG. 9A). As a result, cancer protection, antigen processing in tumor DCs, frequency of tumor-infiltrating, antigen-specific T cells, as well as cytokine production and cytotoxicity were all strikingly reduced when prosaposin was lacking (FIG. 4B-4E). Altogether, these findings demonstrate that tumor immunity critically depends on prosaposin function.

T Lymphocytes from Melanoma Patients are Boosted by Prosaposin

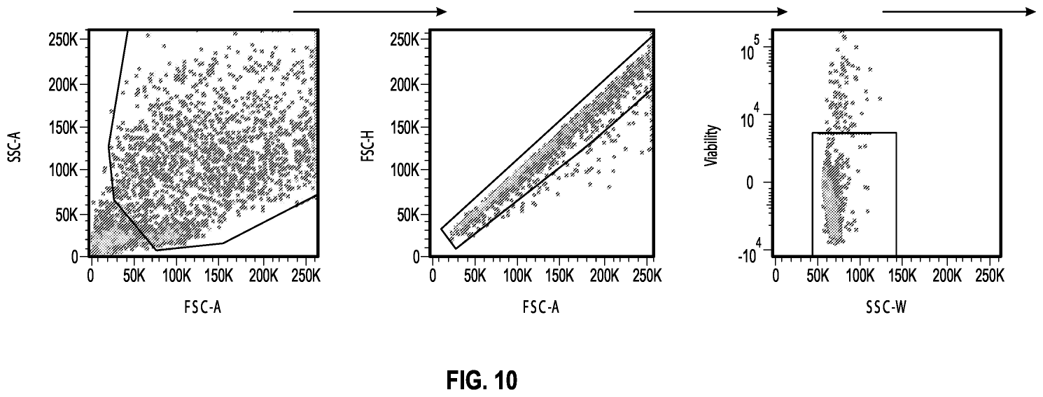

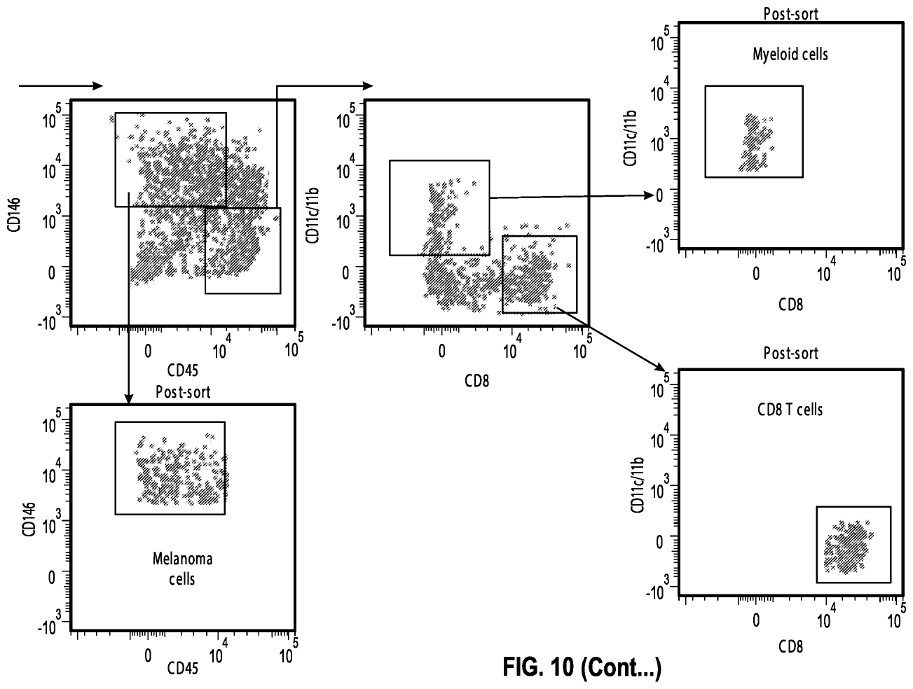

To study prosaposin in the context of human cancer, dissociated tumor cell (DTC) samples were used from melanoma patients. A detailed description of the patient samples can be found in table Si. Briefly, the majority of specimens were isolated from primary melanoma lesions from the skin, were assigned a clinical stage III, before treatment, including white, female and male patients older than 50 years (Table 1). To purify antigen-presenting cells, responder T cells, and tumor cells as source of antigen, CD146⁺ melanoma cells, CD11b/c⁺ myeloid cells, and CD8⁺ T cells were FACS-sorted (FIG. 10). CD146⁺ melanoma cells were then irradiated and pulsed them onto sorted myeloid cells, prior to coculture with autologous CD8 T cells (FIG. 4E). In parallel, DCs and T cells were also treated with human recombinant prosaposin. Five days after culture, effector functions of CD8 T cells were analyzed and found that recombinant prosaposin was able to boost IFN-γ production (FIG. 4F), as well as cytolytic activity as indicated by surface LAMP-1 staining as sign of cytotoxic degranulation (FIG. 4G). Furthermore, the frequencies of tumor antigen-specific CD8 T cells were measured by staining with MHC-I tetramers loaded with dominant melanoma antigens, including MART, gp100, Tyrosinase, and NY-ESO-1. The abundance of melanoma-specific CD8 T cells was strikingly increased when tumor DCs were treated with prosaposin (FIG. 4H). Of note, the patient samples have been HLA-typed by flow cytometry beforehand to select the proper haplotype for MHC-I tetramer analysis (HLA-A02). To conclude, the impact of prosaposin on tumor DCs is able to rescue T cell activation from melanoma patients.

TABLE 1
Description of melanoma patient samples used in this study.

| | | Overall | Overall | Patient | | HLA-A02 |
|---|---|---|---|---|---|---|
| Primary | | clinical | treatment | age at | | by flow |
| diagnosis | Tumor location | stage | status | collection | Gender | cytometry |
| Melanoma | Primary tissue | III-A | Pre Tx | 67 | Male | Positive |
| | from back skin | | | | | |
| Melanoma | Primary tissue | III-C | Pre Tx | 72 | Female | Positive |
| | from back skin | | | | | |
| Melanoma | Primary tissue | III | Pre Tx | 61 | Female | Negative |
| | from neck skin | | | | | |
| Melanoma | Primary tissue | III | Pre Tx | 69 | Female | Negative |
| | from back skin | | | | | |
| Melanoma | Primary tissue | III | Post Tx | 70 | Male | Negative |
| | from skin | | | | | |
| Melanoma | Primary tissue | III-C | Pre Tx | 68 | Female | Negative |
| | from left hip | | | | | |
| Melanoma | Primary tissue | III-C | Pre Tx | 64 | Female | Positive |
| | from skin | | | | | |
| Melanoma | Primary tissue | III | Pre Tx | 57 | Female | Positive |
| | from left arm | | | | | |
| | skin | | | | | |
| Melanoma | Primary tissue | II-B | Pre Tx | 57 | Male | Positive |
| | from left hip | | | | | |
| Melanoma | Primary tissue | III-C | Post Tx | 67 | Male | Positive |
| | from neck skin | | | | | |
| Melanoma | Primary tissue | III | Pre Tx | 62 | Female | Positive |
| | from back skin | | | | | |
| Melanoma | Primary tissue | III | Pre Tx | 65 | Female | Positive |
| | from neck skin | | | | | |

### Hyperglycosylation of Prosaposin in Tumor DCs Leads to its Secretion

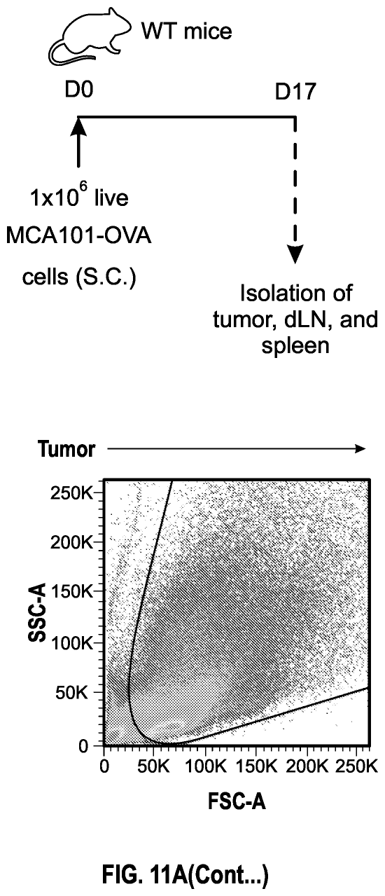

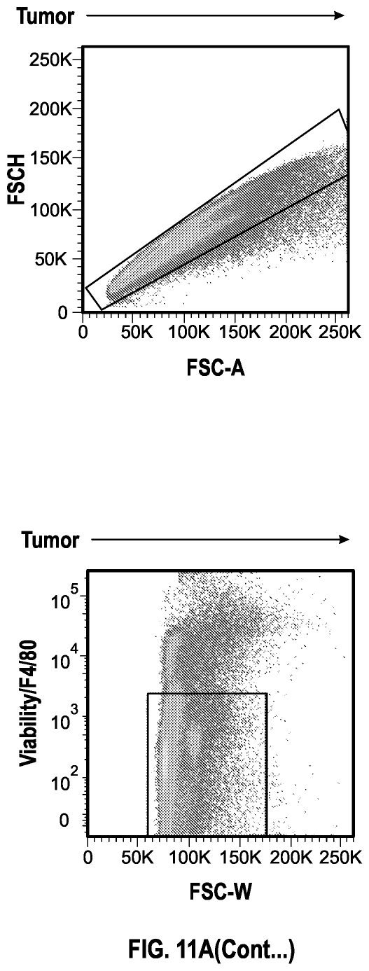

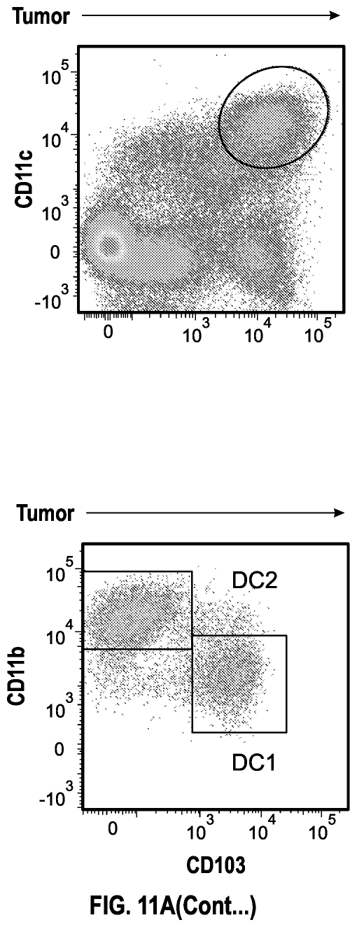

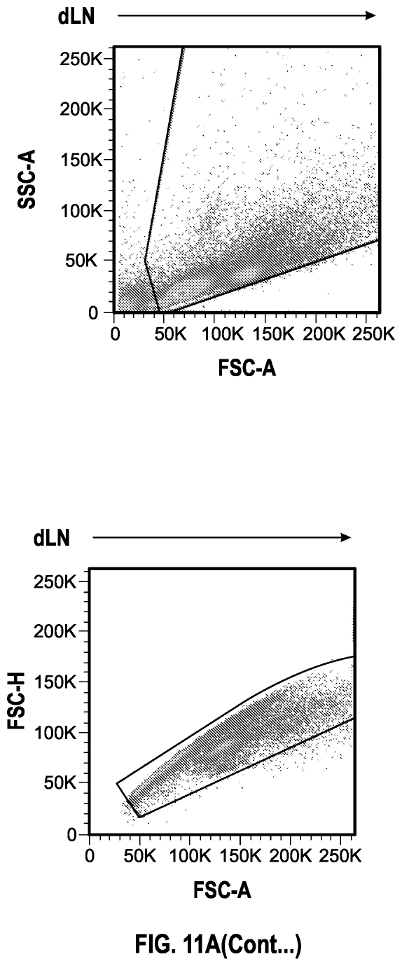

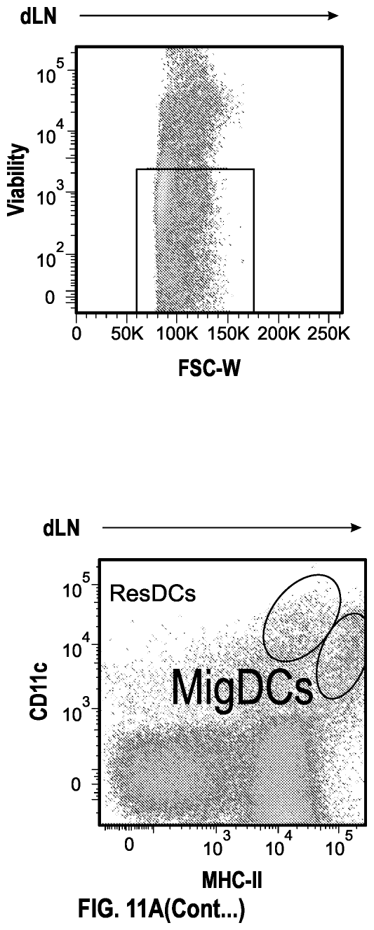

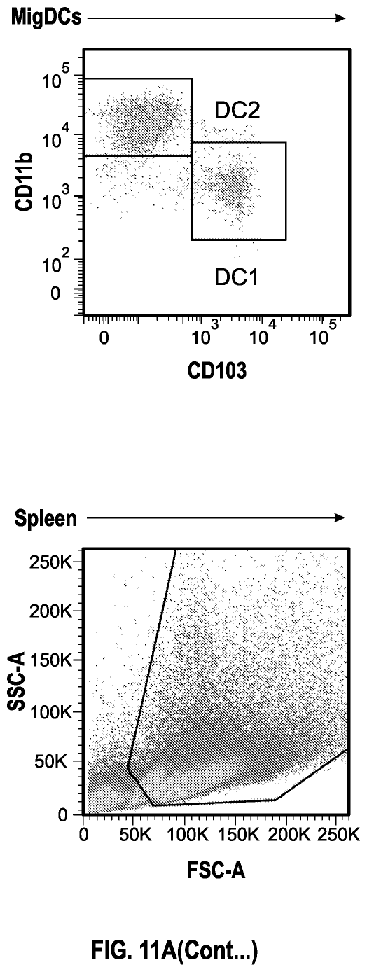

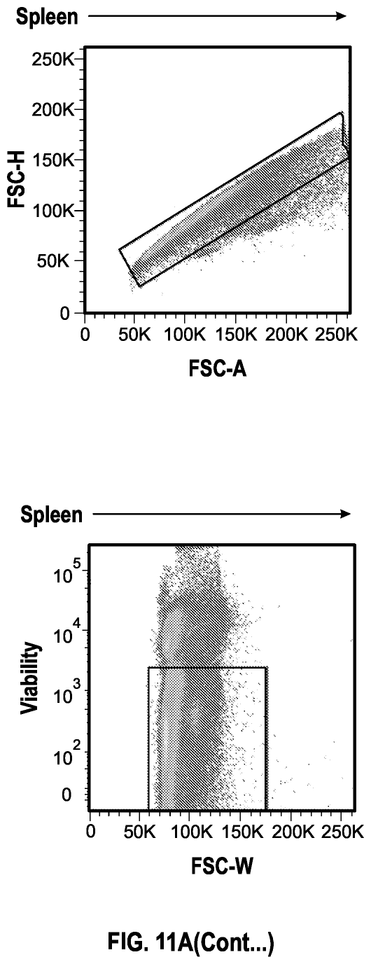

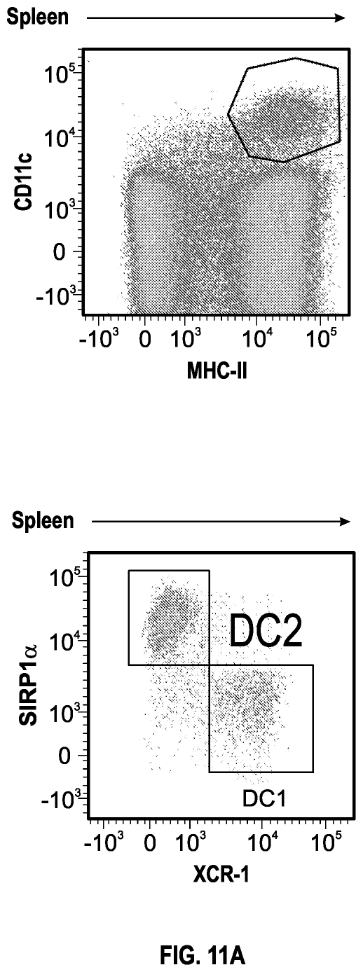

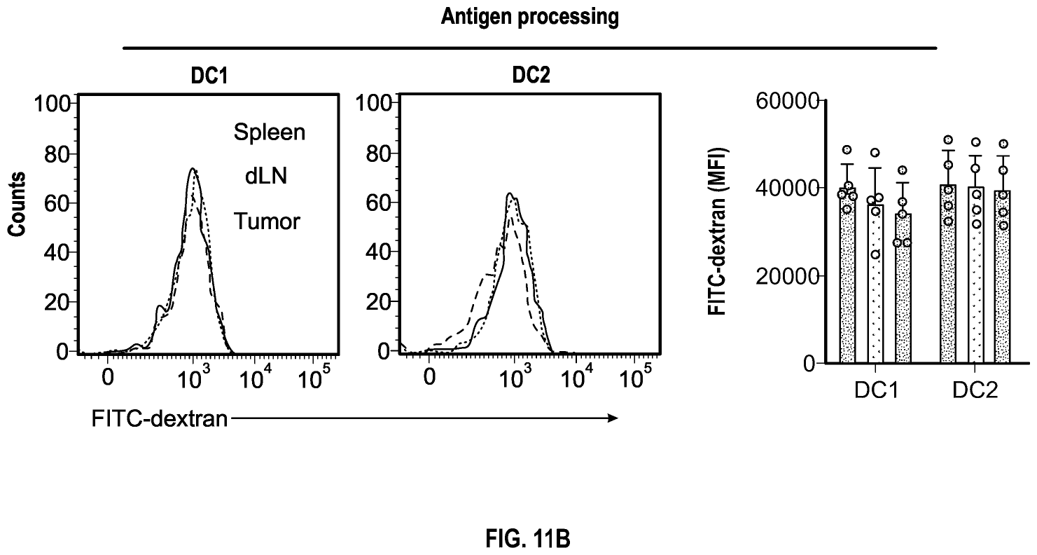

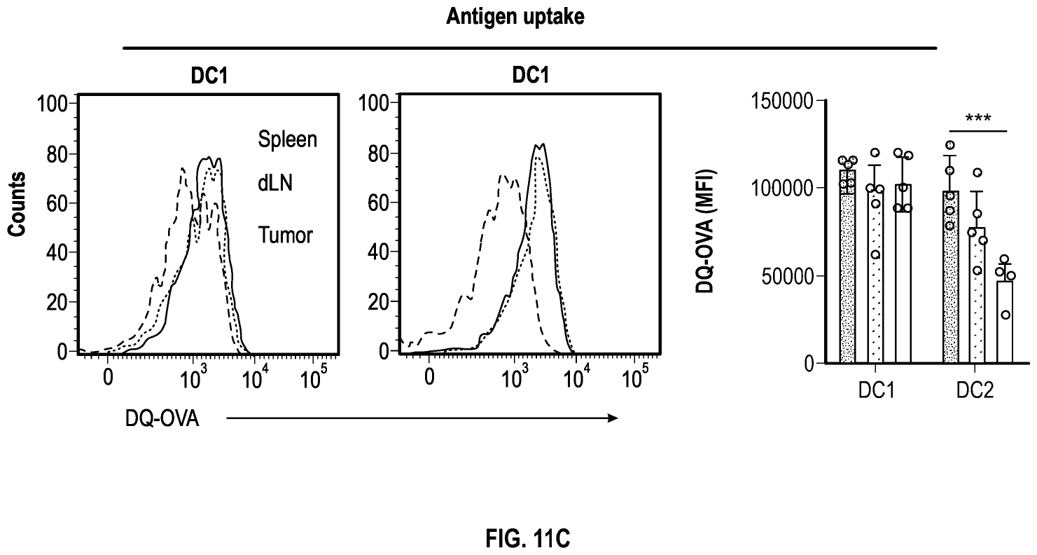

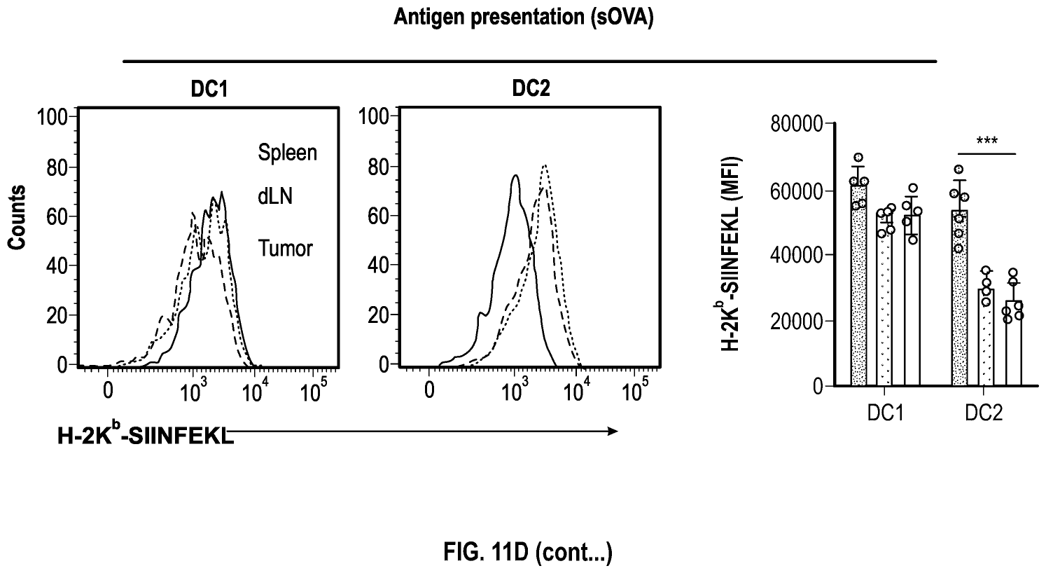

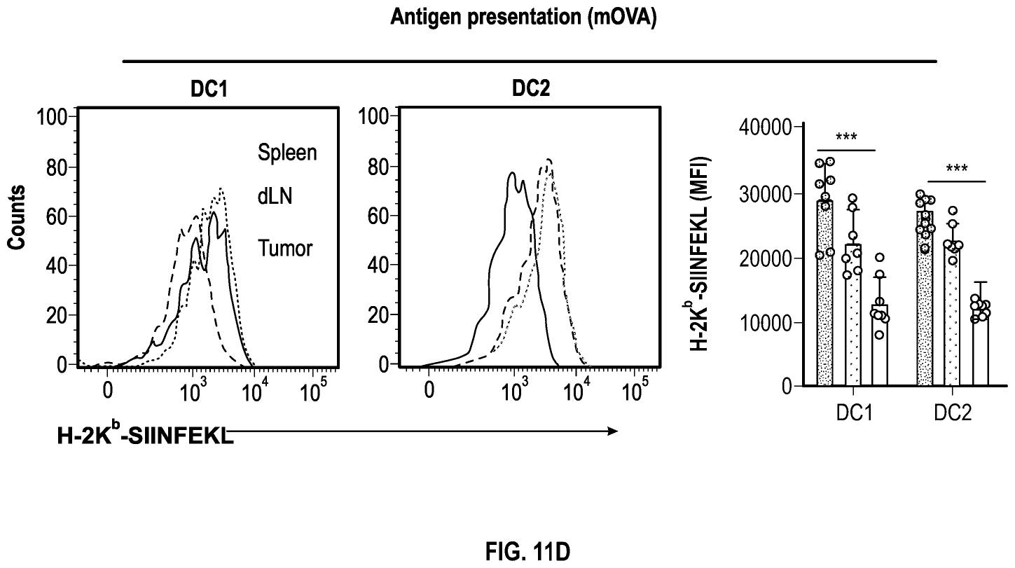

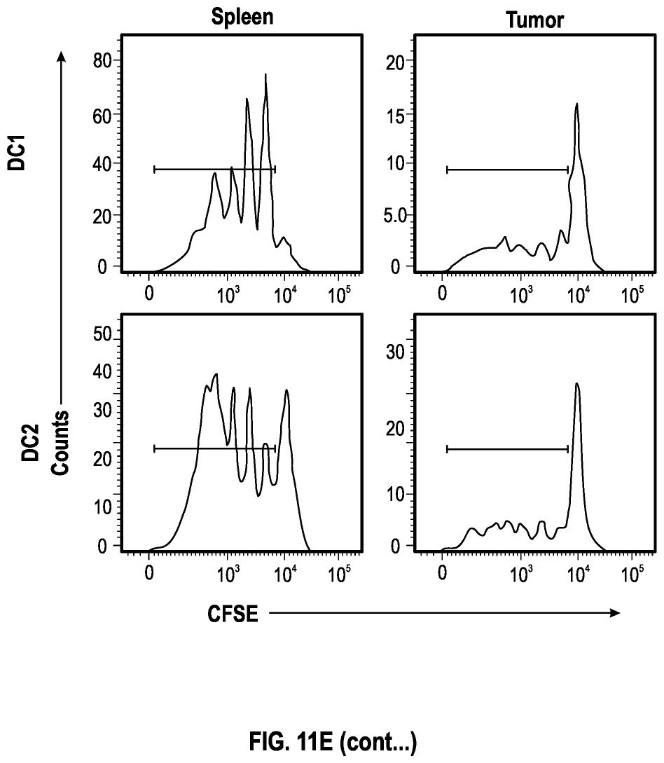

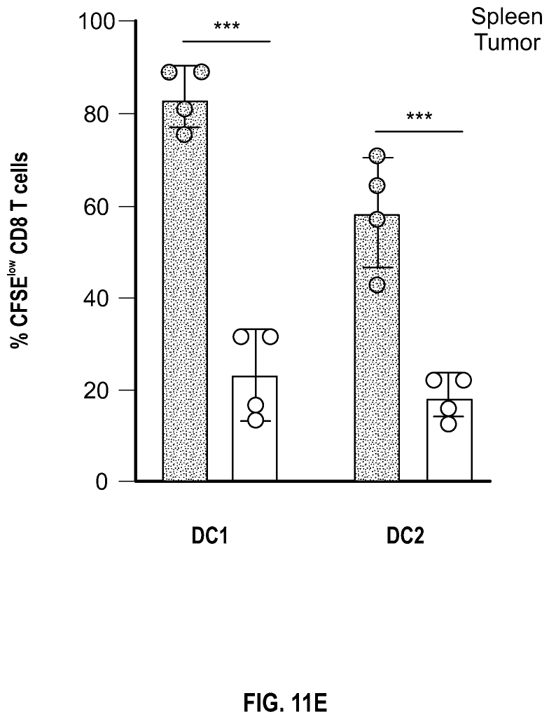

Beyond the use of pSAP deficiency in a mouse model, the regulation of prosaposin in tumor-associated DCs was investigated in a pathophysiological context. To this end, WT mice were inoculated with live MCA101-OVA cells subcutaneously and subsequent to tumor outgrowth, DCs were isolated from the tumor microenvironment (TME), tumor-draining lymph nodes, and spleen (FIG. 11A). The two main classical DC subsets were FACS-purified based on their established markers as cDC1 (XCR1⁺) and cDC2 (SIRP1α⁺), prior to performing an array of antigen processing and presentation assays. Accordingly, pulsing with FTC-dextran showed that the phagocytosis rate of tumor DCs was not altered when compared to lymph node and spleen (FIG. 11B). Incubation with a self-quenched antigen conjugate (DQ-OVA), which exhibits fluorescence upon proteolytic degradation, demonstrated that mainly cDC2 in the tumor are compromised to process soluble antigen (FIG. 11C). These findings were in line with the ultimate epitope expression on surface MHC-I following pulsing with soluble OVA, which revealed hampered antigen presentation by tumor cDC2 (FIG. 11D). In sharp contrast, the presentation capacity of cDC1 in the TME was only affected after incubation with irradiated MCA101-OVA cells, which contain antigen in membrane-associated form (FIG. 11D). This phenomenon was reflected in functional T cell experiments, because DCs isolated from tumors were severely perturbed to induce T cell responses reactive to membrane-associated antigen (FIG. 11E). Since it was found that saposins are critical for presentation of particulate antigen, it was hypothesized that prosaposin function might be modulated in tumor DCs as a basis for poor T cell induction in the TME.

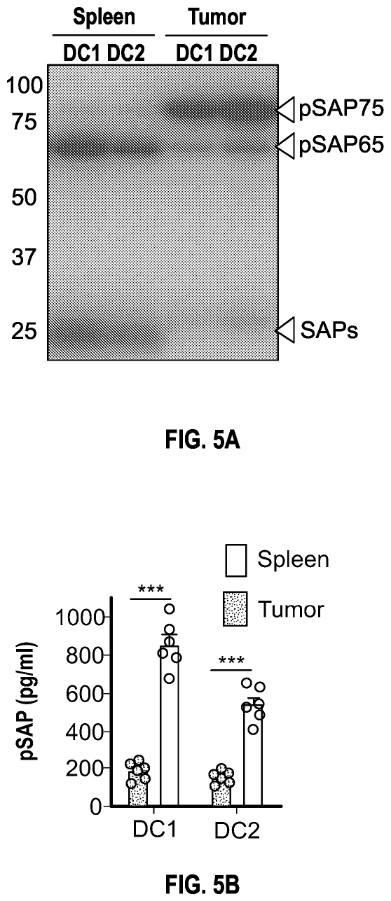

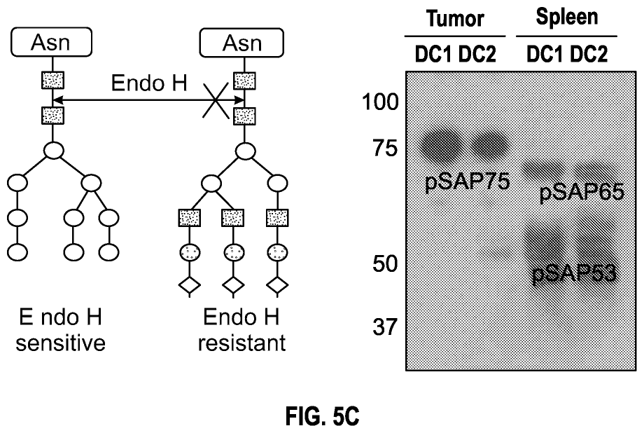

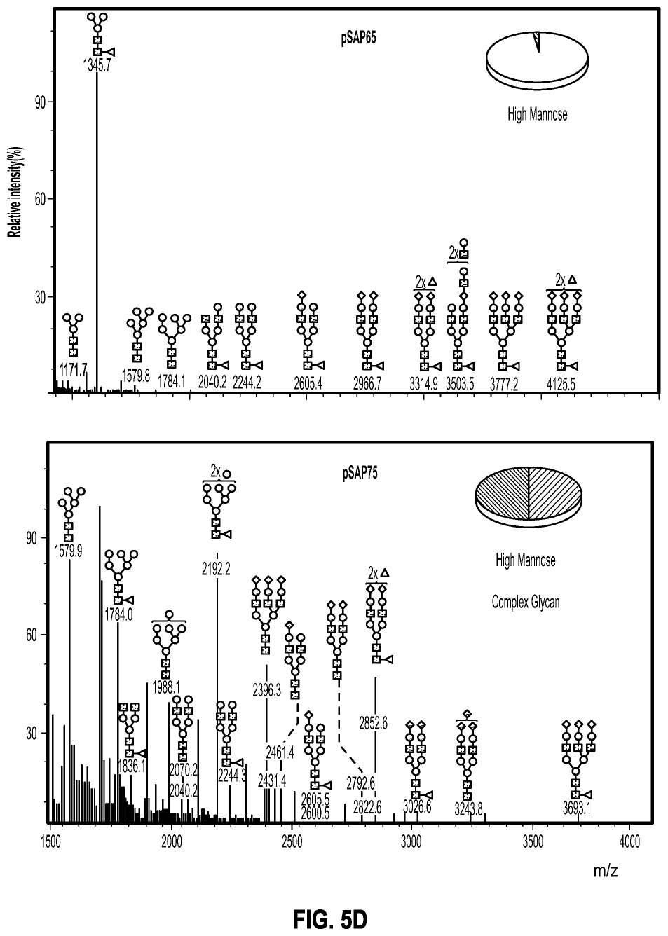

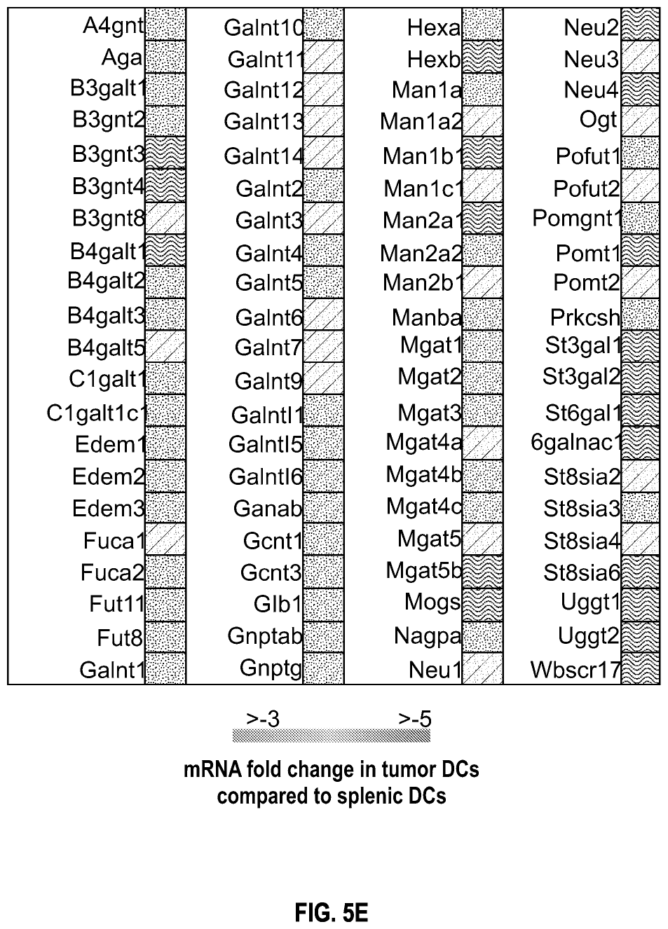

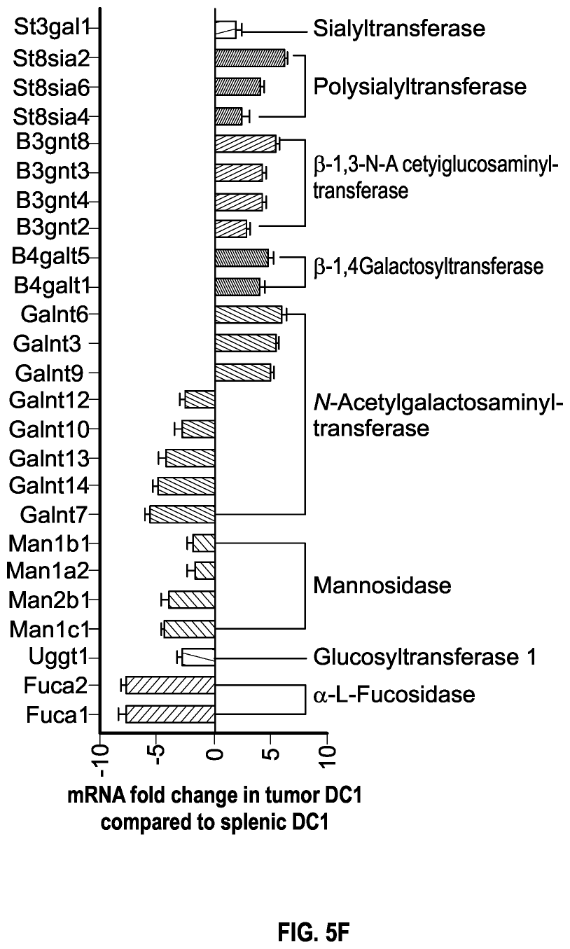

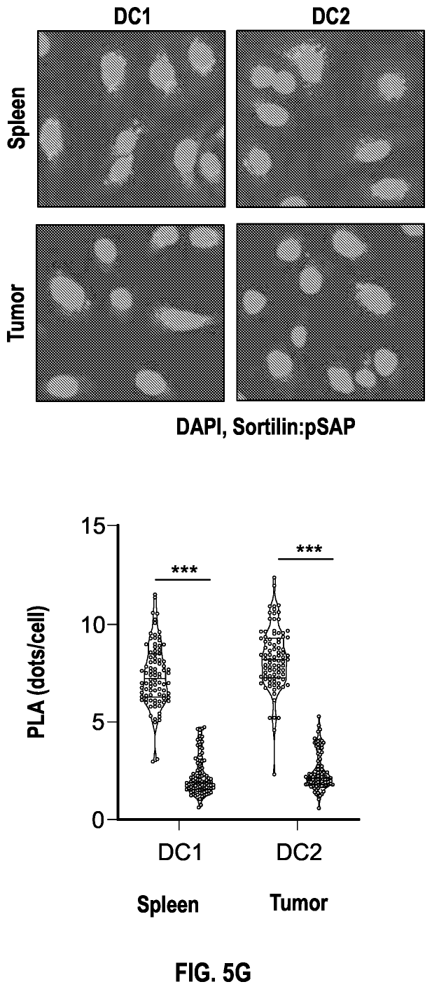

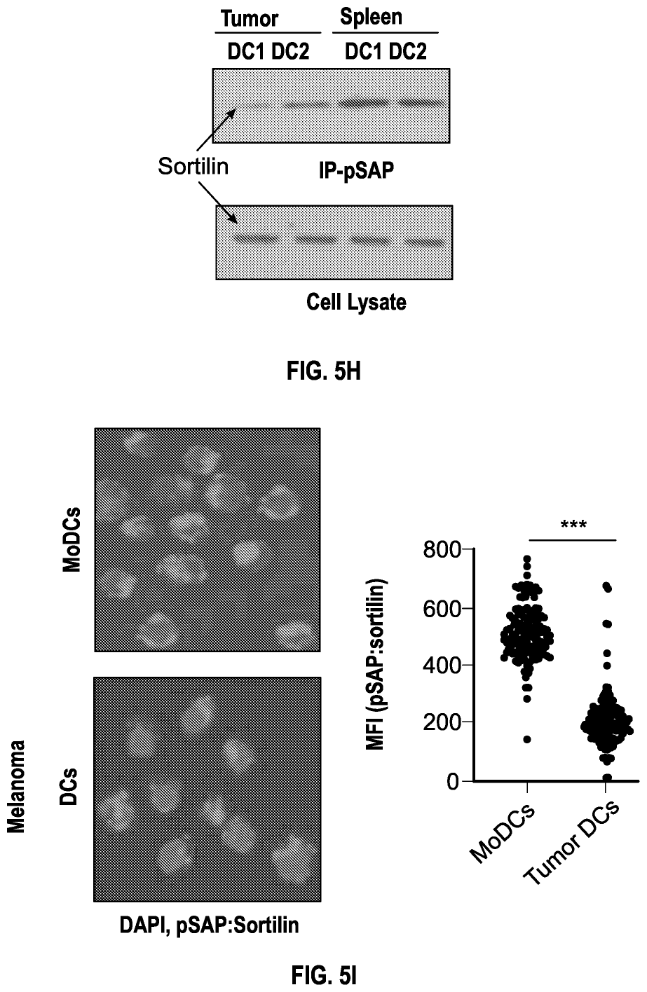

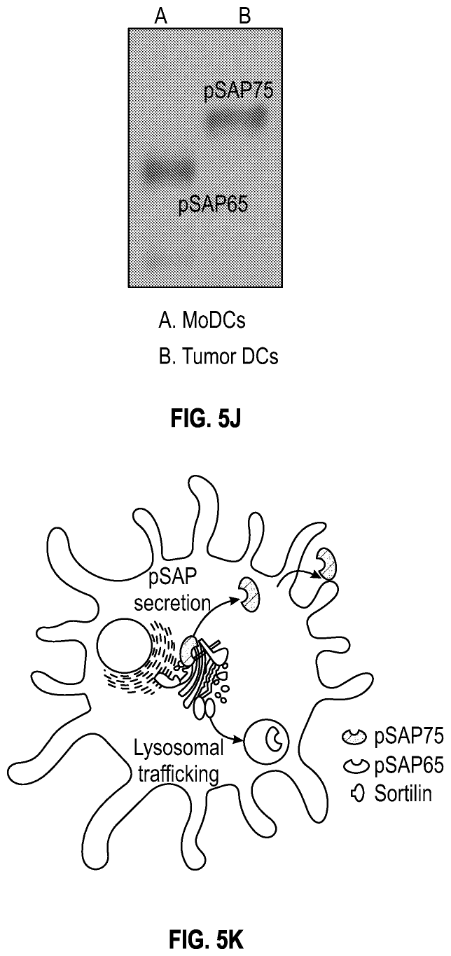

Indeed, analysis by immunoblot revealed the expression of a 75 kDa high-molecular weight form of prosaposin in tumor DCs when compared to pSAP-65 predominant in DCs from spleen (FIG. 5A). Moreover, the small, single saposins were severely depleted in DCs from the TME (FIG. 5A). When the respective DC subsets were cultured ex vivo, secretion of prosaposin was observed into the cell culture supernatant mainly by tumor DCs as measured by ELISA (FIG. 5B). In order to demonstrate that the occurrence of pSAP-75 was due to glycosylation, endoglycosidase H (Endo H) sensitivity of prosaposin was tested. Endo H cleaves N-linked glycans between the two proximal N-acetylglucosamine residues only in high-mannose carbohydrate chains, but not in complex glycans. After treatment of protein lysates with Endo H, pSAP-65 from splenic DCs was cleaved to lower molecular weight forms, whereas pSAP-75 from tumor DCs proved to be endo H-resistant, suggesting that it contained complex glycans (FIG. 5C). To corroborate this finding, deeper molecular analysis was performed using mass spectrometry of sugar structures based on purified pSAP bands derived from tumor DCs. Tandem mass spectrometry showed that the glycan of pSAP-65 mainly consisted of mannose residues, while pSAP-75 exhibited complex glycans involving additions of N-acetylglucosamine, galactose, and sialic acid (FIG. 5D). Since glycan structures are synthesized by a diverse set of glycosyltransferases, the expression of glycosyltransferases was compared between tumor and splenic DCs using a qRT-PCR array (FIG. 5E). In tumor DCs, upregulation of several enzymes was found that facilitate the attachment of complex glycan residues, such as N-acetylglucosaminyltransferases, galactosyltransferases, and sialyltransferases (FIG. 5F).

Since hyperglycosylation of prosaposin leads to its secretion and therefore reduced generation of single saposins, the interaction of pSAP with its chaperone sortilin was investigated, which is normally required for efficient lysosomal delivery of prosaposin. For this purpose, tumor and splenic DCs were subjected to a proximity ligation assay (PLA), in which antibodies against pSAP and sortilin are coupled to oligonucleotide probes that allow for subsequent ligation and amplification in case the two target proteins are in close vicinity (10-80 nm). Confocal microscopy revealed these PLA events as discrete spots and showed that the spatial relationship between prosaposin and sortilin was perturbed in DCs from the TME (FIG. 5G). To assess the direct physical interaction between pSAP and sortilin via biochemistry, immunoprecipitation of prosaposin was performed and subsequently resolved sortilin using immunoblot. As a result, the abundance of sortilin recovered from the pSAP precipitate was clearly reduced in tumor DCs, indicating that the interaction of prosaposin with its chaperone sortilin is hampered in the TME (FIG. 5H). To translate these findings to the human system, pSAP/sortilin interaction was explored in DCs isolated from the tumor site of melanoma patients compared to monocyte-derived DCs. The resulting PLA signals demonstrated that the spatial interaction of prosaposin with sortilin was reduced in melanoma DCs (FIG. 5I). Furthermore, the abundance of the different molecular weight forms of prosaposin was examined and found that tumor DCs from melanoma patients exclusively expressed hyperglycosylated pSAP-75 (FIG. 5J). Taken together, the results demonstrate that prosaposin is hyperglycosylated in tumor DCs, fails to interact with its chaperone sortilin, and follows instead a secretory route (FIG. 5K). This mechanism of pSAP hyperglycosylation leads to a depletion of intracellular saposins available for antigen processing, which might explain the compromised antigen presentation capacity in the tumor microenvironment.

### TGF-β Induces Prosaposin Hyperglycosylation in Tumor DCs and Drives Immune Evasion

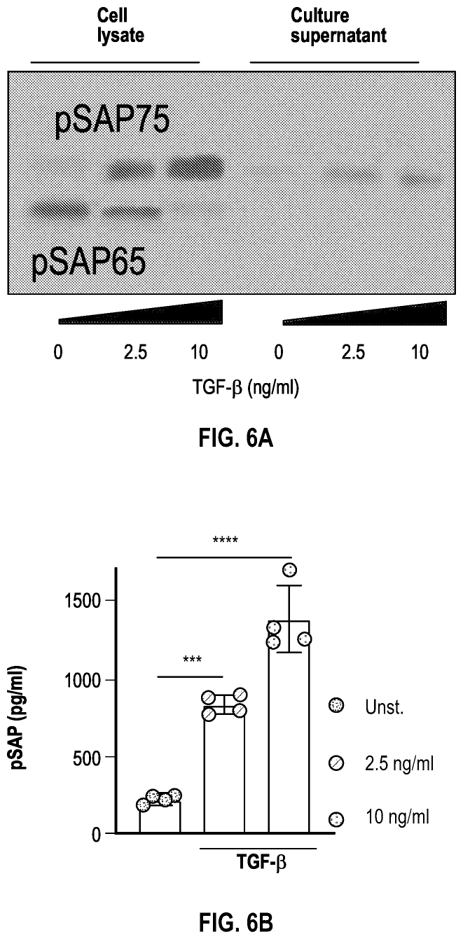

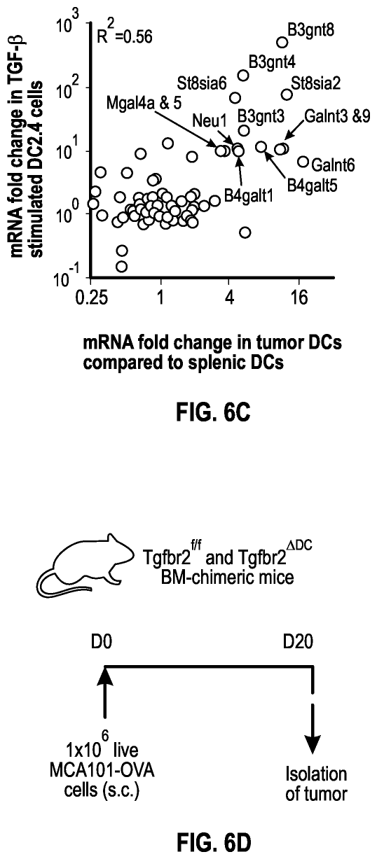

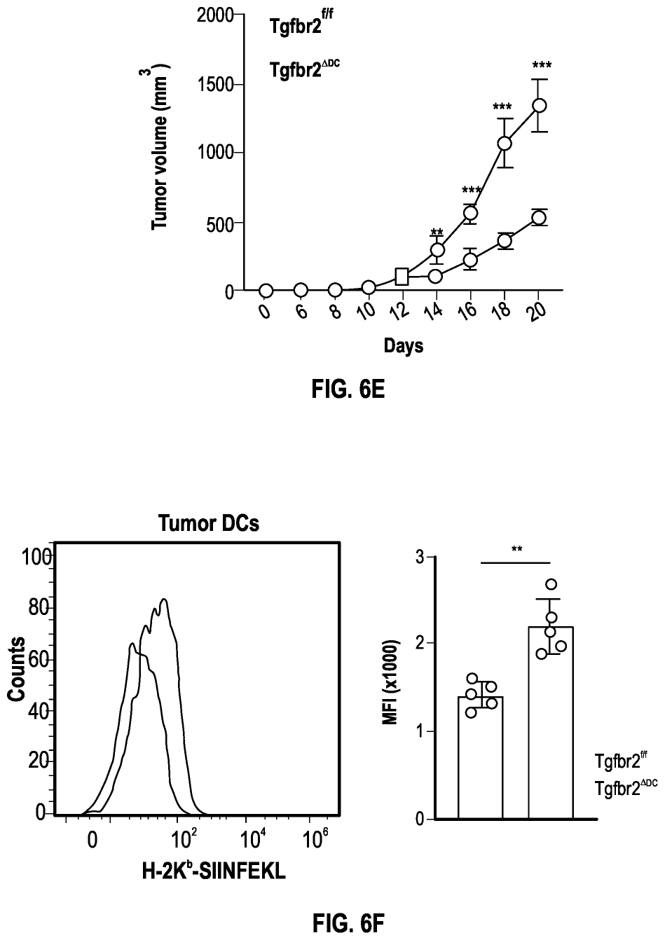

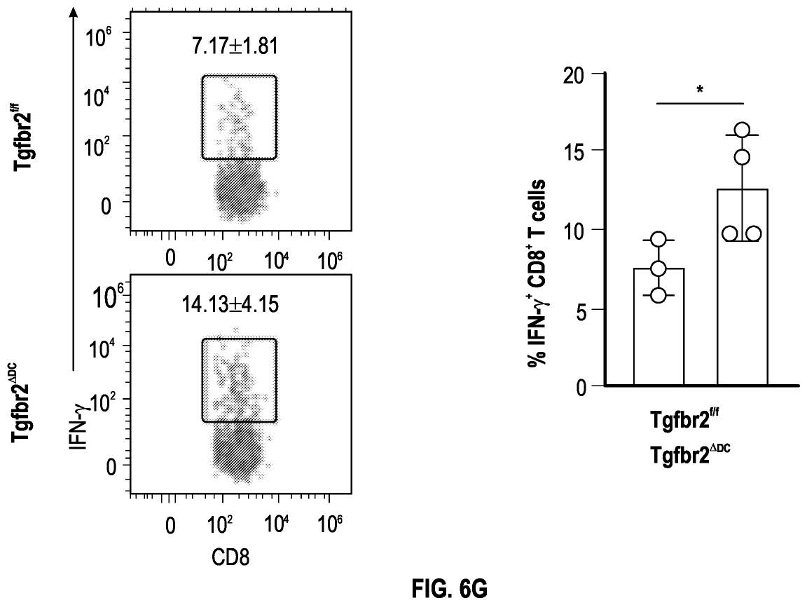

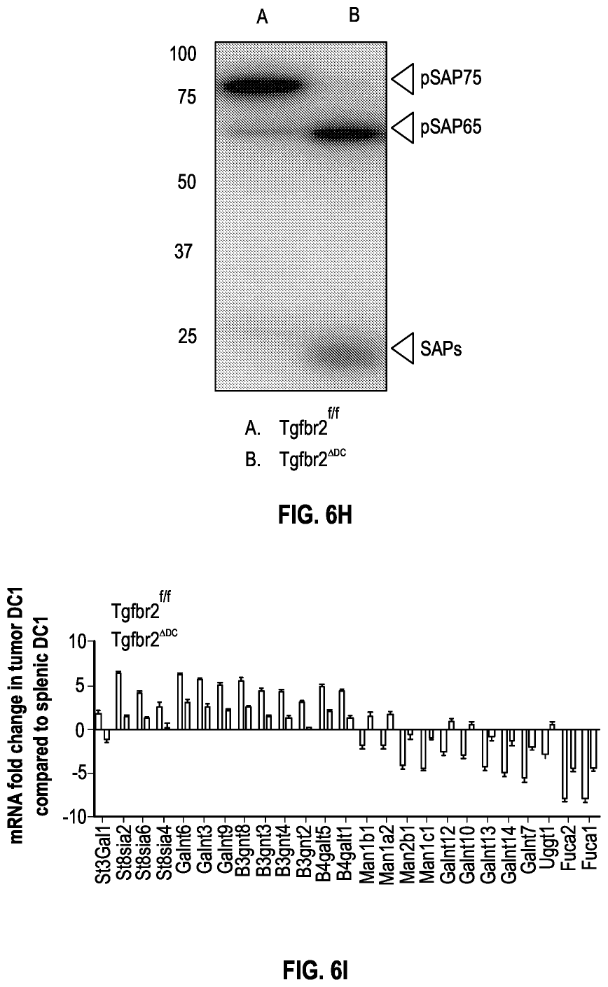

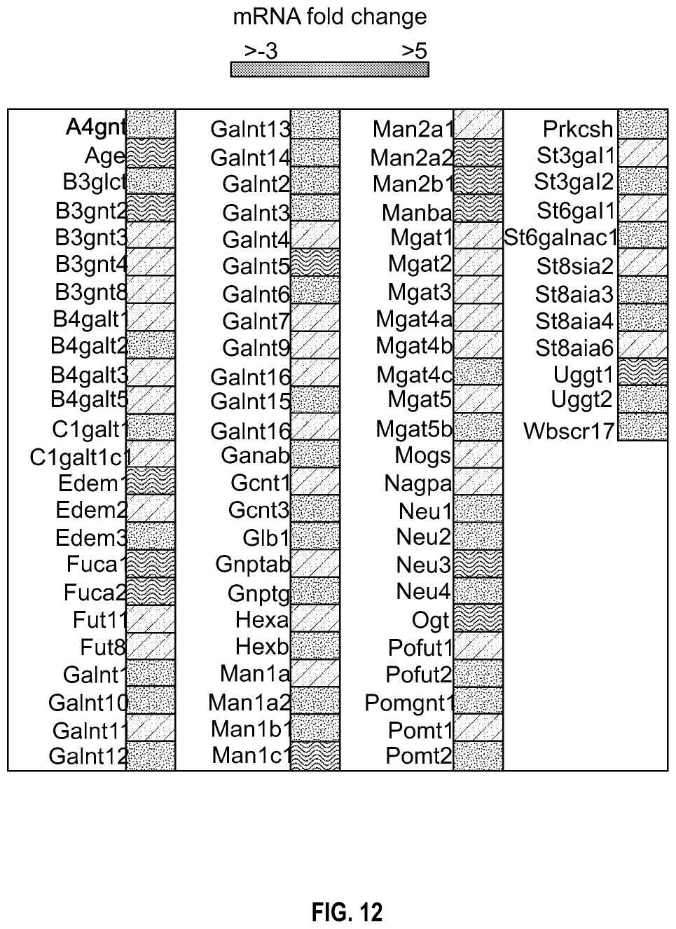

Next, the question how hyperglycosylation of prosaposin is regulated was addressed. Considering its prominent immunosuppressive function as a cytokine, a murine DC line (DC2.4) was incubated with recombinant TGF-β and observed a dose-dependent induction of pSAP-75 (FIG. 6A). In addition, treatment with TGF-β was able to trigger secretion of pSAP-75 into the cell culture supernatant as detected by immunoblot and ELISA (FIGS. 6A-6B). Moreover, gene expression of a panel of enzymes involved in the glycosylation pathway was measured in DC2.4 cells treated with TGF-β (FIG. 12). It was observed that the upregulated genes in TGF-3-treated DCs correlated well with the enzyme signature detected in tumor DCs (FIG. 6C), suggesting that TGF-β is responsible for triggering the respective glycosylation program. In order to test whether TGF-β signaling is indeed required for prosaposin hyperglycosylation in vivo, mice that lack TGF-β receptor II were used specifically in DCs (CD11c-Cre×Tgfbr2^{flox/flox}) for challenge with live MCA101-OVA tumor cells (FIG. 6D). As anticipated, lack of TGF-β downstream signaling in DCs caused better tumor protection, increased antigen presentation in tumor DCs, and stronger IFN-γ production by tumor-infiltrating CD8 T cells (FIGS. 6E-6G). More importantly, when tumor DCs were isolated for analysis by immunoblot, it was found that pSAP-75 was virtually absent in DCs lacking TGF-β signaling (FIG. 6H). Additionally, tumor DCs lacking TGF-β receptor exhibited an abundance of single saposins, which was in striking contrast to the saposin depletion and expression of hyperglycosylated prosaposin predominating over pSAP-65 in WT DCs (FIG. 6H). Furthermore, the enzyme signature involved in glycosylation proved to be reduced when TGF-β signaling was deficient in tumor DCs (FIG. 6I). Thus, TGF-β is essential for hyperglycosylation of prosaposin in tumor DCs, a mechanism associated with immune escape.

Immunotherapeutic Targeting of Tumor DCs with Recombinant Prosaposin

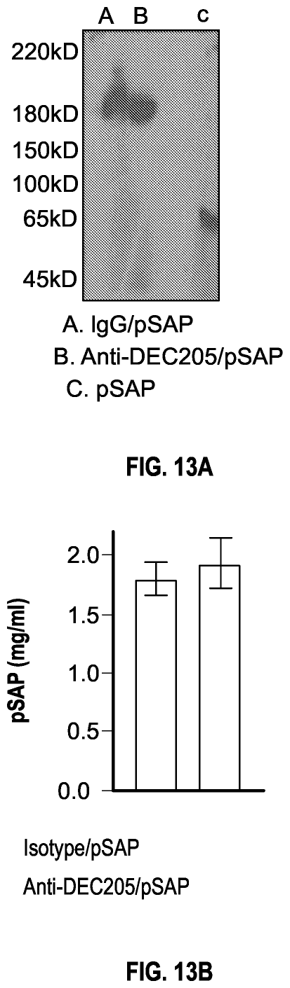

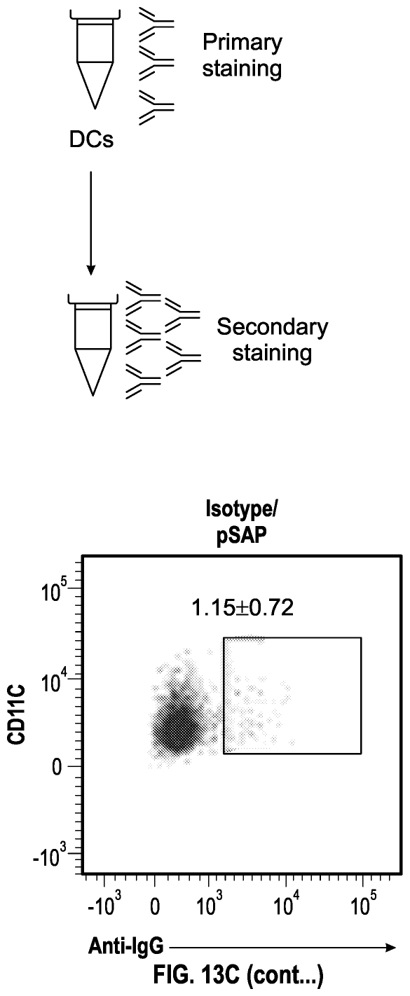

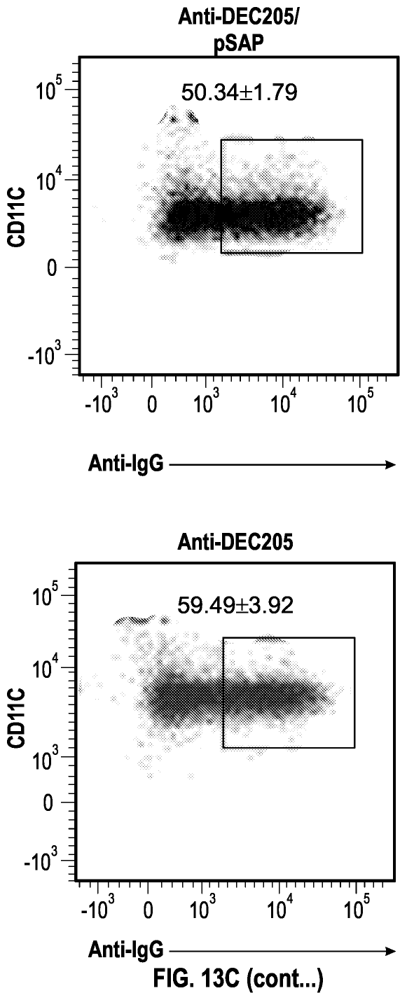

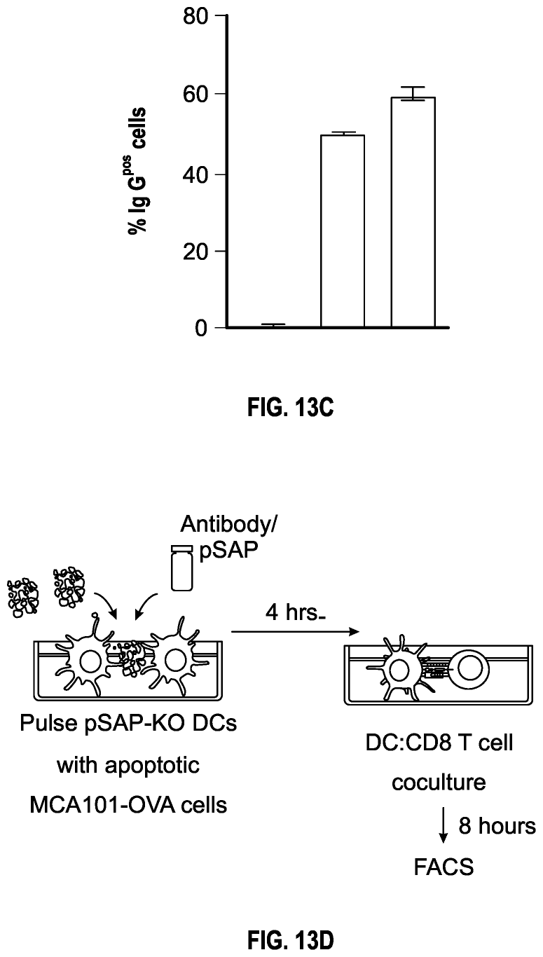

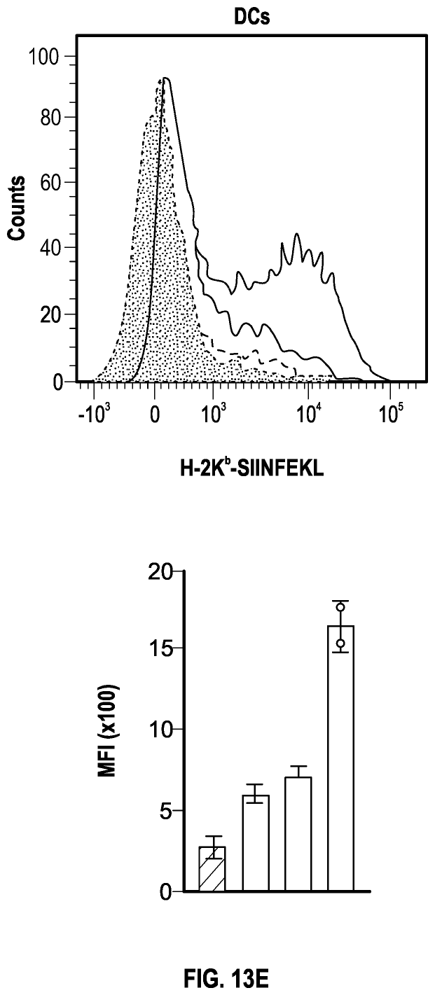

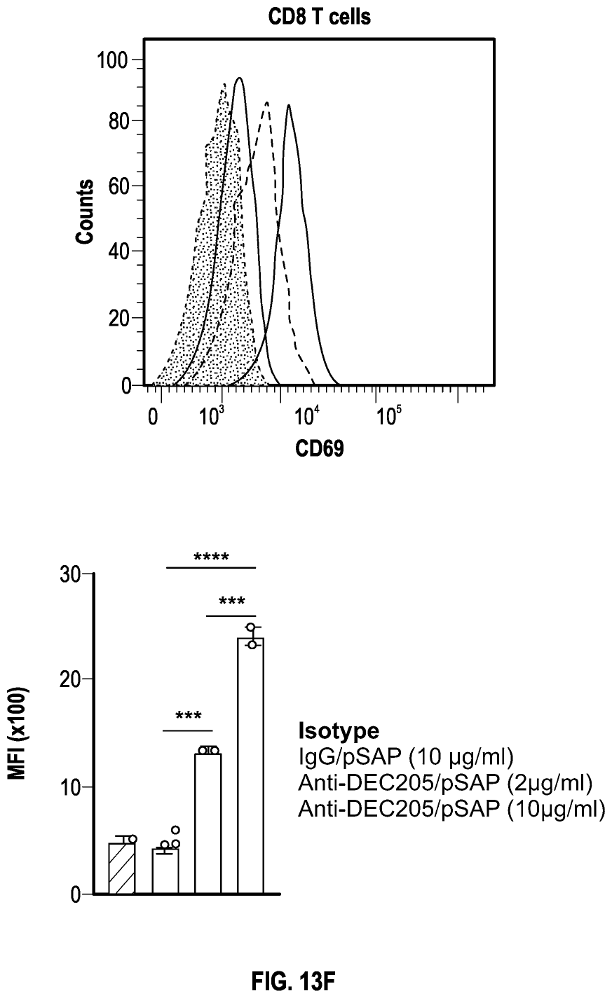

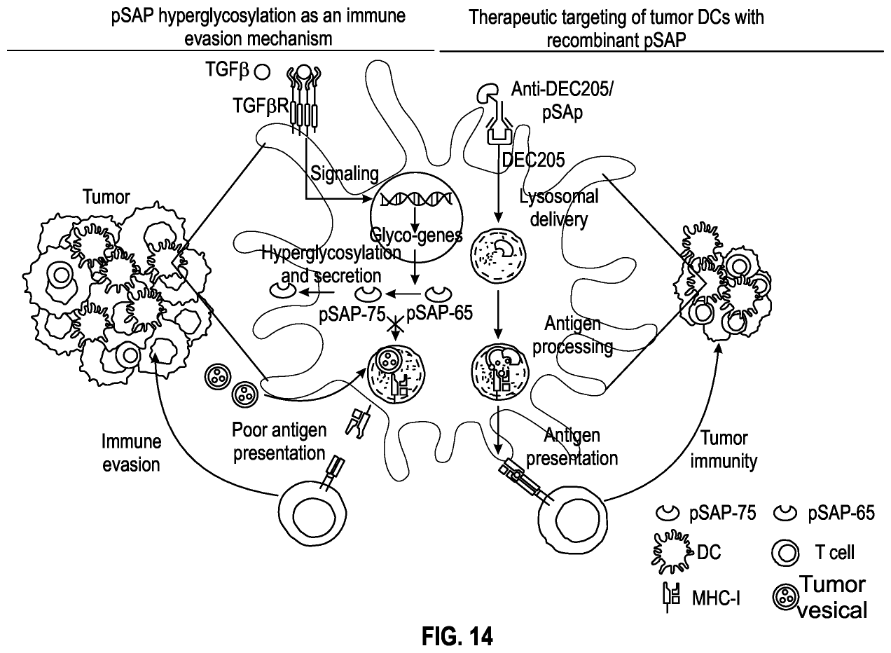

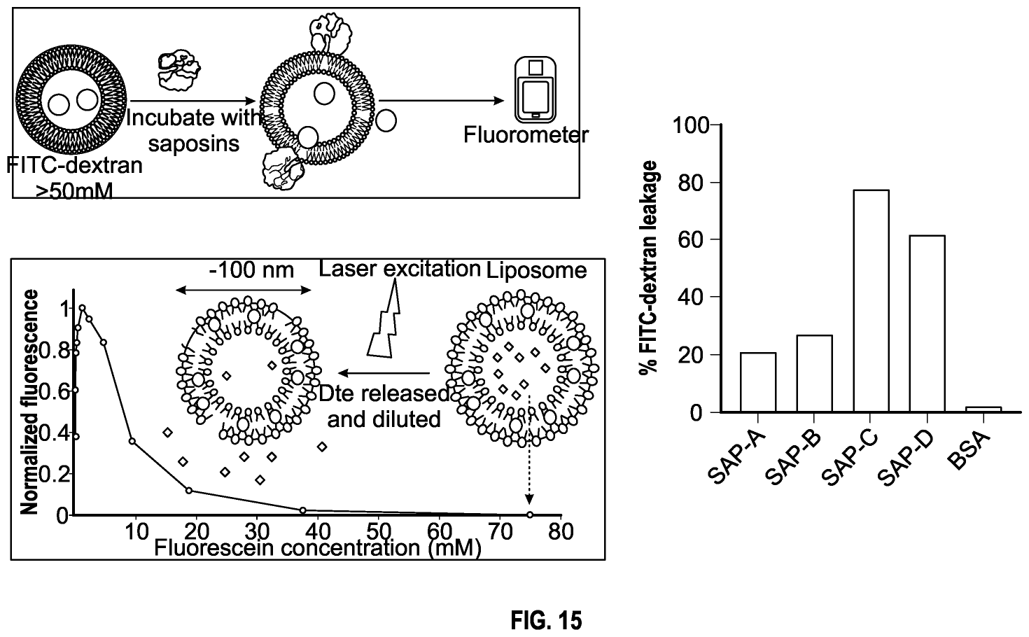

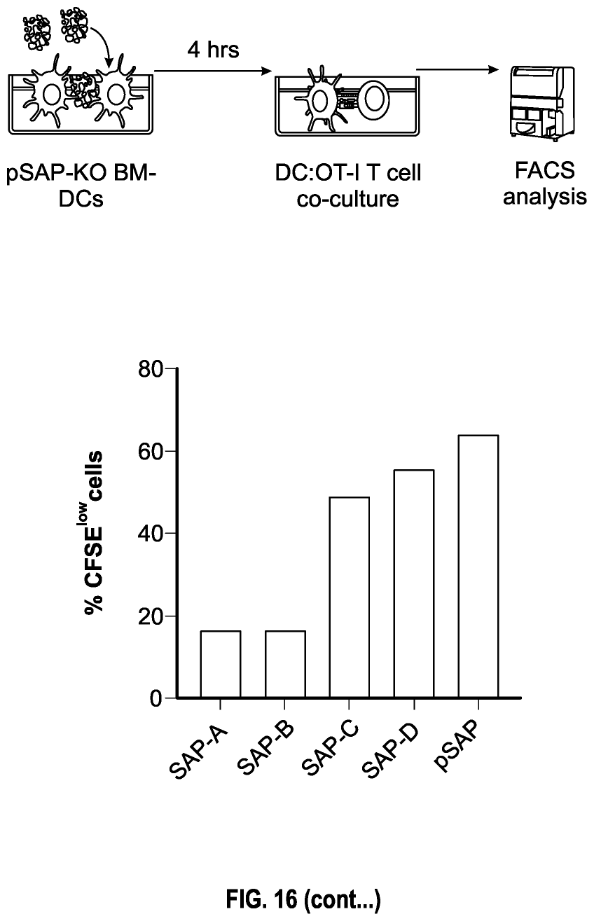

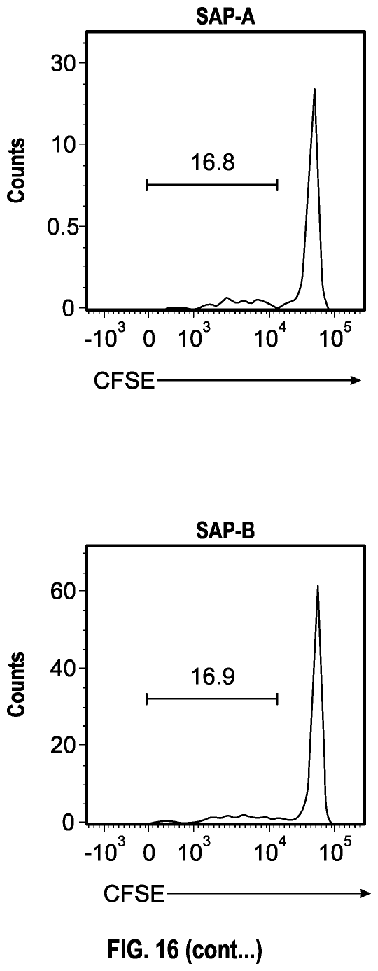

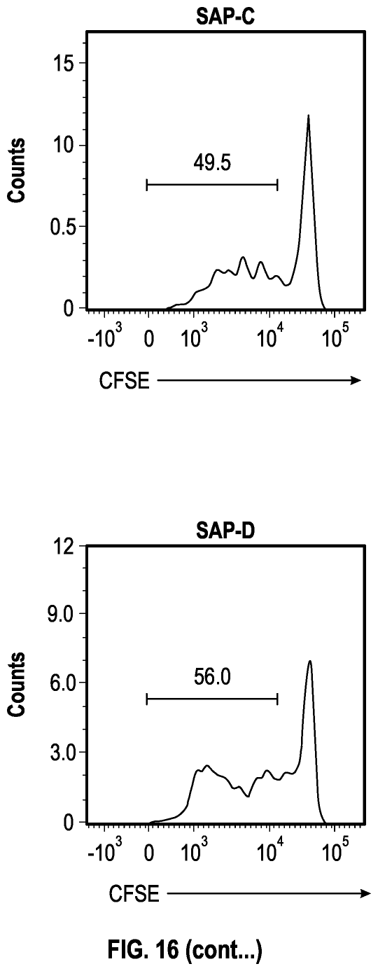

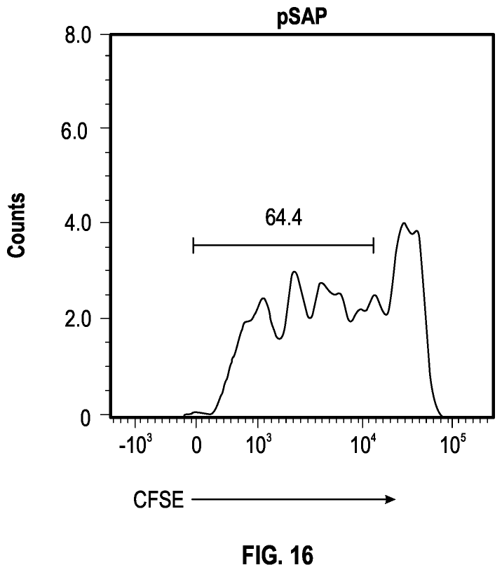

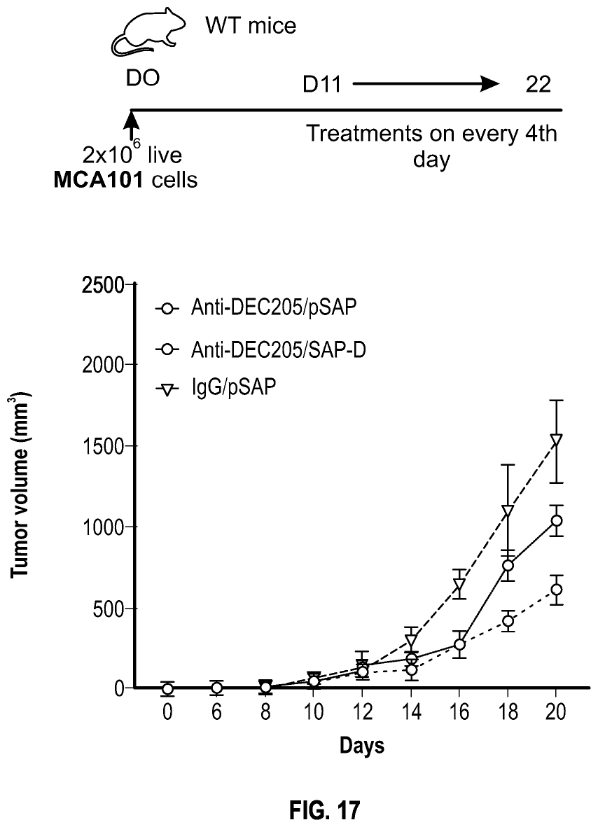

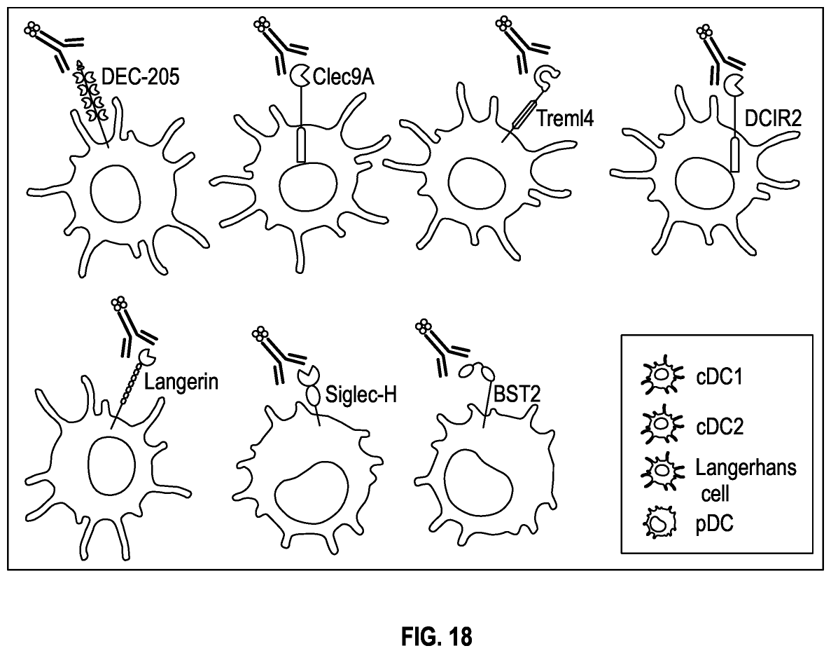

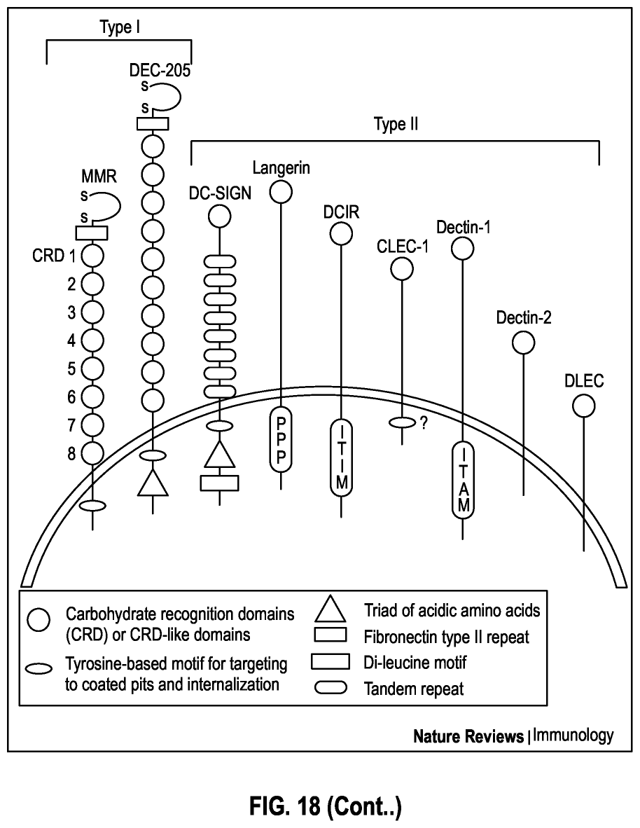

Based on the importance of prosaposin for antigen presentation in the tumor microenvironment, recombinant prosaposin was targeted to tumor DCs. For this purpose, prosaposin was coupled to anti-DEC205, an antibody well established to target the endocytic receptor DEC205 on DCs, using chemical conjugation as previously described. Briefly, recombinant prosaposin was incubated with TCEP-HCl (tris (2-carboxyethyl) phosphine hydrochloride) in order to expose its sulfhydryl groups, and in parallel, the anti-DEC205 antibody or isotype control IgG were activated for chemical conjugation by sulfo-SMCC (sulfosuccinimidyl 4-[N-maleimidomethyl] cyclohexane-1-carboxylate). Following overnight incubation, the prosaposin/antibody conjugates were concentrated and successful coupling analyzed by immunoblot (FIG. 13A). In addition, the amount of controlled prosaposin coupled to anti-DEC205 or isotype control was comparable (FIG. 13B). Moreover, it was verified that prosaposin conjugation still preserved the fine specificity of the DEC205 antibody by showing that the pSAP/anti-DEC205 conjugate stained a similar percentage of DCs when compared to separate anti-DEC205 detection using flow cytornetry (FIG. 13C). Furthermore, pSAP-KO DCs was incubated with the prosaposin/antibody conjugates and tested OVA epitope expression and CD8 T cell activation after pulsing of DCs with apoptotic MCA101-OVA cells (FIG. 13D). Accordingly, reconstitution of antigen presentation (H-2k^{b}-SIINFEKL) was observed and CD8 T cell priming (CD69) specifically by prosaposin coupled to anti-DEC205 in a dose-dependent manner (FIGS. 13E-13F).

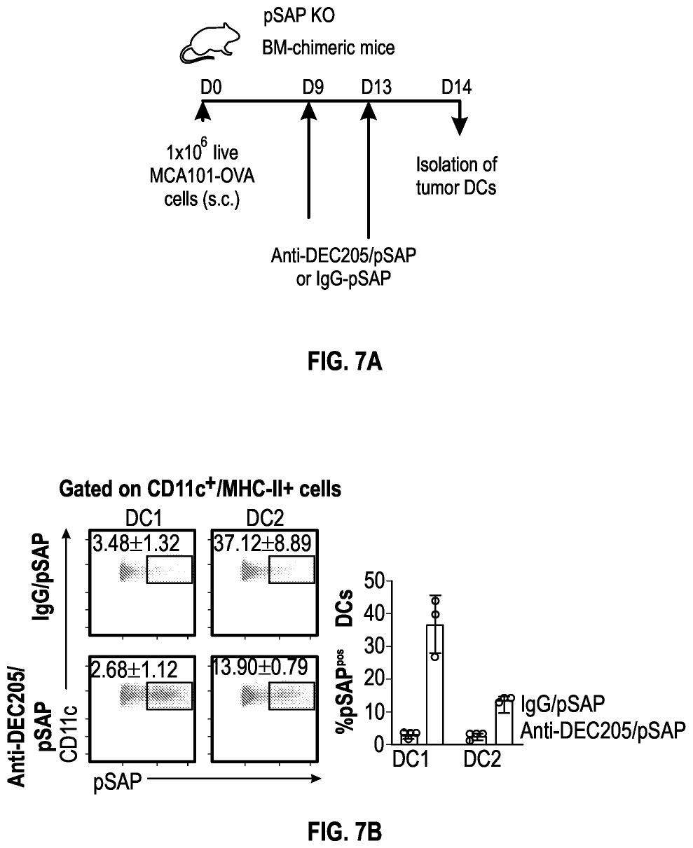

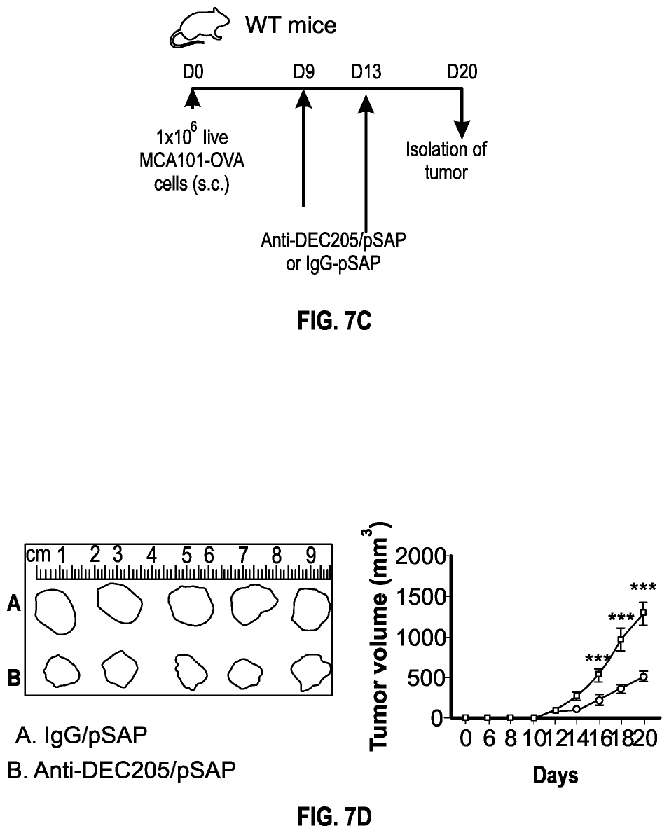

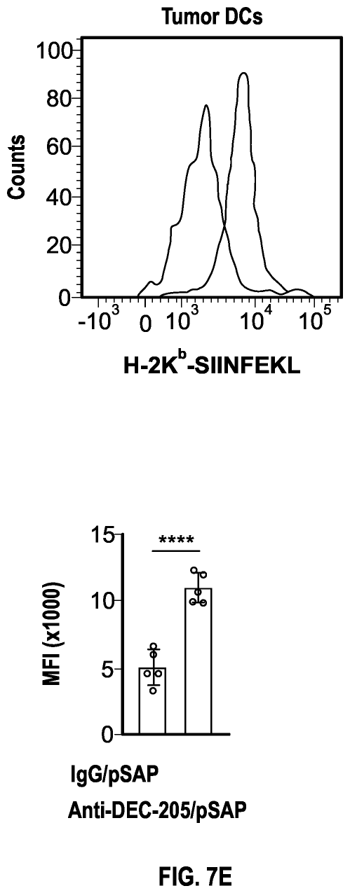

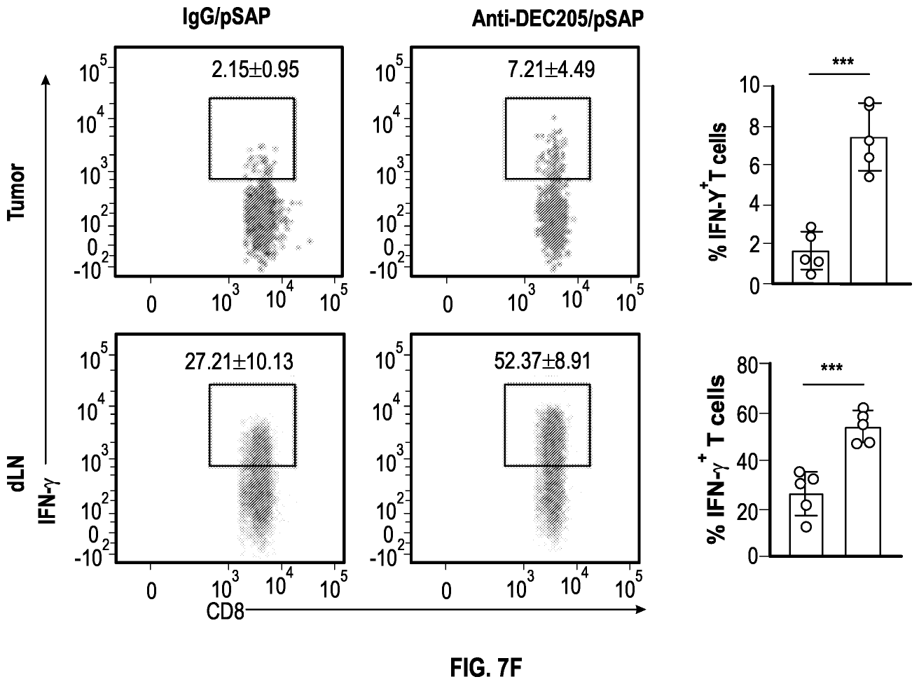

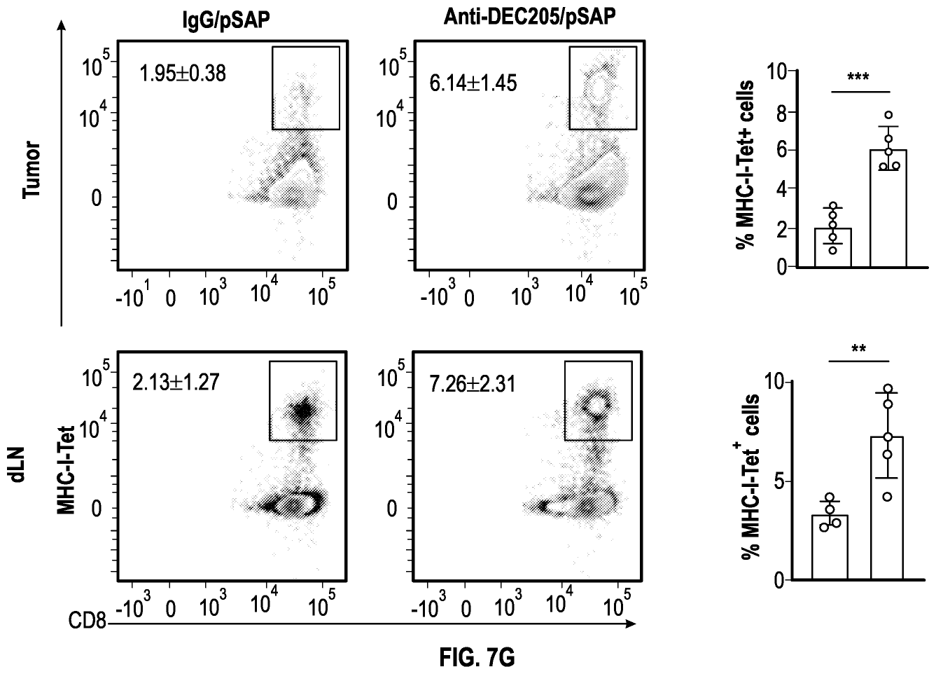

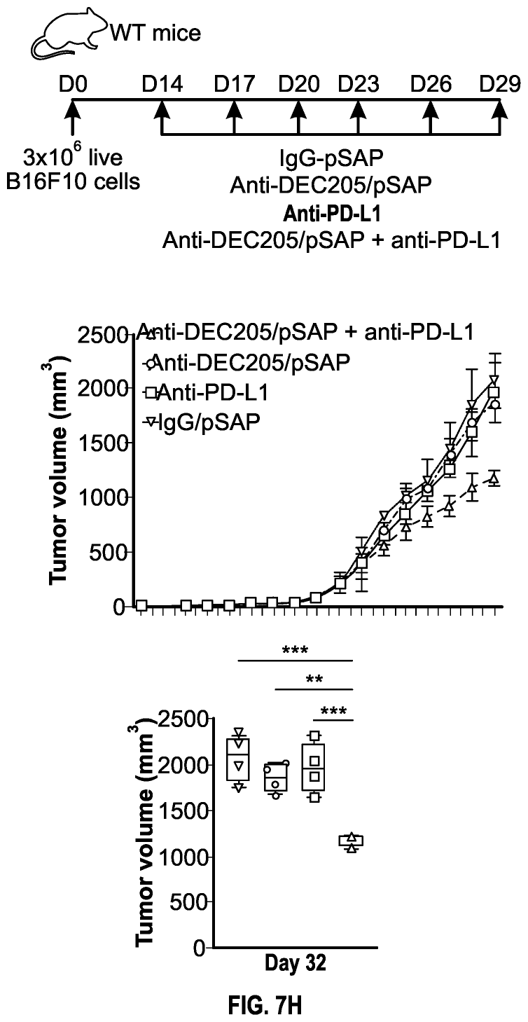

After validation of the targeting tool in vitro and ex vivo, pSAP-KO BM chimeric mice were inoculated with MCA101-OVA tumor cells and injected 100 g prosaposin coupled to anti-DEC205 or isotype IgG on day 9 and 13 after tumor challenge (FIG. 7A). On day 14, intra-tumoral DCs were isolated and sorted, focusing on the two major classical DC subsets cDC1 and cDC2. Staining for intracellular prosaposin in the respective DC subsets revealed effective delivery of pSAP targeted via DEC205 when compared to isotype control (FIG. 7B). This demonstrated successful reconstitution of pSAP-deficient DCs in the tumor microenvironment, especially in the cDC1 subtype that is central to cross-priming of CD8 T cells. Next, a similar experimental schedule was followed using pSAP targeting of tumor-inoculated WT animals (FIG. 7C). Treatment with pSAP coupled to anti-DEC205 strikingly reduced tumor burden in WT mice (FIG. 7D). Correspondingly, antigen presentation of OVA peptide by tumor DCs as well as frequency of IFN-γ-producing, antigen-specific T cells at the tumor site and in draining lymph nodes (dLNs) were significantly amplified upon pSAP treatment (FIGS. 7E-7G). While pSAP delivery to tumor DCs promoted an immunologically active TME, it was next asked whether prosaposin can also rescue immune suppression in immunologically cold tumors. For this purpose, we used the B16F10 melanoma model that displays limited T cell infiltration and low susceptibility to treatment with immune checkpoint inhibitors, such as anti-PD-1, despite strong expression of PD-L1. When compared to mice treated with anti-PD-L1 alone, the tumor growth kinetics revealed that pSAP combination therapy was able to overcome the resistance of B16F10 melanoma to immune checkpoint blockade in order to enable protection (FIG. 7H). Taken together, these results highlight the crucial impact of prosaposin on antigen presentation by tumor DCs to trigger powerful intra-tumoral T cell responses and point to a viable future strategy for immunotherapy of cancer.

### Discussion

Herein, the critical function of saposins is demonstrated in processing of corpuscular, membrane-associated antigen required for efficient CD8 T cell activation. Notably, saposins were not essential for cross-presentation of soluble antigen, which mechanistically involves rather an early endosomal or phagosomal compartment. In cancer immunity, the provision of particulate antigen derived from dying tumor cells represents a physiologic and potent route of antigen delivery. In this context, it has been shown that only DCs that contain tumor-derived vesicles are able to induce T cell responses. Thus, the findings highlight the importance of the lysosome in cross-priming of T cells and underscore the impact of saposins on the immunogenic pathway of vesicular processing.

In tumor biology, previous reports suggested a trophic function of prosaposin able to stimulate proliferation of cancer cells. However, prosaposin and a pSAP-derived synthetic cyclic peptide were able to prevent tumor metastasis in a mouse model. The work established a genuine immunological function of prosaposin in tumor DCs. Thus, pSAP-driven antigen processing and presentation at the tumor site amplified abundance and functionality of tumor-infiltrating T lymphocytes, ultimately leading to cancer control.

TGF-β is a pleiotropic cytokine with a diverse set of immunosuppressive functions and is also produced by most cell types. Therefore, tumors themselves as well as the infiltrating cells of the tumor microenvironment can serve as source and target of TGF-β. For example, tumor-derived TGF-β is capable of restricting T cell infiltration or to functionally block differentiation of protective T lymphocyte populations. The results revealed that TGF-β acts on tumor DCs to trigger hyperglycosylation of prosaposin and its subsequent secretion, depleting the lysosomal pool of saposins required for proper antigen processing and presentation. The secretion of prosaposin has been shown to be caused by pSAP oligomerization in a cell line. However, in primary DCs in the tumor microenvironment, large oligomers were not observed and instead found perturbed interaction between sortilin and hyperglycosylated prosaposin. An additional lysosomal player, called progranulin, might be involved in this mechanism, as it has been shown to bridge the interaction between pSAP and sortilin. Overall, tumors are prone to immune escape, and the work describes a further cancerous strategy to manipulate an antigen processing molecule via glycosylation.

Since hyperglycosylation of prosaposin occurs along the secretory pathway, an approach to overcome tumor-induced saposin deficiency entails the feeding of recombinant pSAP into the endocytic route of DCs. In this context, the targeting experiments using prosaposin coupled to anti-DEC205 demonstrated that reconstitution with fully functional pSAP is able to restore antigen presentation in tumor DCs, leading to amplified T cell responses and eventual tumor protection. DCs are not only crucial for T cell priming in draining lymph nodes. A growing body of evidence indicates that tumor-associated DCs have vital functions with regard to recruitment of effector T cells and stimulation of tumor-infiltrating T lymphocytes, overall required for effective immunity to cancer. Current therapeutic options, such as immune checkpoint blockade, aim at reinvigorating exhausted T cells to augment anti-tumor responses. However, these boosted T lymphocytes need to encounter their respective antigens in order to deploy their functions in a tumor-specific manner. Consistent with that notion, pSAP combination treatment was shown to be able to override the resistance of immunologically cold tumors to immune checkpoint inhibitors. Taken together, a prosaposin-based therapy could help to restore powerful antigen presentation by tumor DCs with the goal to drive protective immune responses at the site of the cancer.

### REFERENCES

1. M. D. Vesely, M. H. Kershaw, R. D. Schreiber, M. J. Smyth, Natural innate and adaptive immunity to cancer. Annu Rev Immunol 29, 235-271 (2011).
2. M. S. Rooney, S. A. Shukla, C. J. Wu, G. Getz, N. Hacohen, Molecular and genetic properties of tumors associated with local immune cytolytic activity. Cell 160, 48-61 (2015).
3. M. Sade-Feldman et al., Resistance to checkpoint blockade therapy through inactivation of antigen presentation. Nat Commun 8, 1136 (2017).
4. A. Alloatti et al., Critical role for Sec22b-dependent antigen cross-presentation in antitumor immunity. J Exp Med 214, 2231-2241 (2017).
5. J. S. Blum, P. A. Wearsch, P. Cresswell, Pathways of antigen processing. Annu Rev Immunol 31, 443-473 (2013).
6. J. M. Blander, Regulation of the Cell Biology of Antigen Cross-Presentation. Annu Rev Immunol 36, 717-753 (2018).
7. L. Shen, L. J. Sigal, M. Boes, K. L. Rock, Important role of cathepsin S in generating peptides for TAP-independent MHC class I crosspresentation in vivo. Immunity 21, 155-165 (2004).
8. L. Saveanu et al., IRAP identifies an endosomal compartment required for MHC class I cross-presentation. Science 325, 213-217 (2009).
9. T. L. Tang-Huau et al., Human in vivo-generated monocyte-derived dendritic cells and macrophages cross-present antigens through a vacuolar pathway. Nat Commun 9, 2570 (2018).
10. K. Hildner et al., Batf3 deficiency reveals a critical role for CD8alpha+ dendritic cells in cytotoxic T cell immunity. Science 322, 1097-1100 (2008).
11. S. T. Ferris et al., cDC1 prime and are licensed by CD4(+) T cells to induce anti-tumour immunity. Nature 584, 624-629 (2020).
12. T. F. Gajewski, H. Schreiber, Y. X. Fu, Innate and adaptive immune cells in the tumor microenvironment. Nat Immunol 14, 1014-1022 (2013).
13. S. K. Wculek et al., Dendritic cells in cancer immunology and immunotherapy. Nat Rev Immunol 20, 7-24 (2020).
14. R. A. Flavell, S. Sanjabi, S. H. Wrzesinski, P. Licona-Limon, The polarization of immune cells in the tumour environment by TGFbeta. Nat Rev Immunol 10, 554-567 (2010).
15. S. Mariathasan et al., TGFbeta attenuates tumour response to PD-L1 blockade by contributing to exclusion of T cells. Nature 554, 544-548 (2018).
16. S. Lefrancois, J. Zeng, A. J. Hassan, M. Canuel, C. R. Morales, The lysosomal trafficking of sphingolipid activator proteins (SAPs) is mediated by sortilin. EMBO J 22, 6430-6437 (2003).
17. L. Yuan, C. R. Morales, A stretch of 17 amino acids in the prosaposin C terminus is critical for its binding to sortilin and targeting to lysosomes. J Histochem Cytochem 58, 287-300 (2010).
18. A. Darmoise, P. Maschmeyer, F. Winau, The immunological functions of saposins. Adv Immunol 105, 25-62 (2010).
19. T. Kolter, K. Sandhoff, Principles of lysosomal membrane digestion: stimulation of sphingolipid degradation by sphingolipid activator proteins and anionic lysosomal lipids. Annu Rev Cell Dev Biol 21, 81-103 (2005).
20. F. Ciaffoni et al., Saposin D solubilizes anionic phospholipid-containing membranes. J Biol Chem 276, 31583-31589 (2001).
21. F. Winau et al., Apoptotic vesicles crossprime CD8 T cells and protect against tuberculosis. Immunity 24, 105-117 (2006).
22. W. Jiang et al., The receptor DEC-205 expressed by dendritic cells and thymic epithelial cells is involved in antigen processing. Nature 375, 151-155 (1995).
23. J. Volckmar et al., Chemical Conjugation of a Purified DEC-205-Directed Antibody with Full-Length Protein for Targeting Mouse Dendritic Cells In Vitro and In Vivo. J Vis Exp, (2021).
24. I. J. Fidler, Biological behavior of malignant melanoma cells correlated to their survival in vivo. Cancer Res 35, 218-224 (1975).
25. S. A. Quezada et al., Limited tumor infiltration by activated T effector cells restricts the therapeutic activity of regulatory T cell depletion against established melanoma. J Exp Med 205, 2125-2138 (2008).
26. M. K. Ruhland et al., Visualizing Synaptic Transfer of Tumor Antigens among Dendritic Cells. Cancer Cell 37, 786-799 e785 (2020).
27. Y. Jiang et al., Prosaposin is a biomarker of mesenchymal glioblastoma and regulates mesenchymal transition through the TGF-beta1/Smad signaling pathway. J Pathol 249, 26-38 (2019).
28. S. Ishihara, D. R. Inman, W. J. Li, S. M. Ponik, P. J. Keely, Mechano-Signal Transduction in Mesenchymal Stem Cells Induces Prosaposin Secretion to Drive the Proliferation of Breast Cancer Cells. Cancer Res 77, 6179-6189 (2017).
29. S. Y. Kang et al., Prosaposin inhibits tumor metastasis via paracrine and endocrine stimulation of stromal p53 and Tsp-1. Proc Natl Acad Sci USA 106, 12115-12120 (2009).
30. S. Wang et al., Development of a prosaposin-derived therapeutic cyclic peptide that targets ovarian cancer via the tumor microenvironment. Sci Transl Med 8, 329ra334 (2016).
31. M. Liu et al., TGF-beta suppresses type 2 immunity to cancer. Nature 587, 115-120 (2020).
32. L. Yuan, C. R. Morales, Prosaposin sorting is mediated by oligomerization. Exp Cell Res 317, 2456-2467 (2011).
33. X. Zhou et al., Impaired prosaposin lysosomal trafficking in frontotemporal lobar degeneration due to progranulin mutations. Nat Commun 8, 15277 (2017).
34. H. Salmon et al., Expansion and Activation of CD103(+) Dendritic Cell Progenitors at the Tumor Site Enhances Tumor Responses to Therapeutic PD-L1 and BRAF Inhibition. Immunity 44, 924-938 (2016).
35. S. Spranger, D. Dai, B. Horton, T. F. Gajewski, Tumor-Residing Batf3 Dendritic Cells Are Required for Effector T Cell Trafficking and Adoptive T Cell Therapy. Cancer Cell 31, 711-723 e714 (2017).
36. J. E. Jang et al., Crosstalk between Regulatory T Cells and Tumor-Associated Dendritic Cells Negates Anti-tumor Immunity in Pancreatic Cancer. Cell Rep 20, 558-571 (2017).
37. K. C. Barry et al., A natural killer-dendritic cell axis defines checkpoint therapy-responsive tumor microenvironments. Nat Med 24, 1178-1191 (2018).
38. P. Sharma et al., The Next Decade of Immune Checkpoint Therapy. Cancer Discov 11, 838-857 (2021).

### Materials and Methods

### Mice

Wild-type C57BL/6J, B6.SJL (B6.SJL-Ptprca Pepcb/Boy), OVA-specific OT-1 TCR-transgenic mice (B6.TcraTcrb 1100Mjb/J), CD11c-Cre (B6.Cg-Tg(Itgax-cre)1-1Reiz/J), and floxed TGF-β receptor II mice (B6; 129-Tgfbr2tm1Karl/J) were purchased from Jackson Laboratories. B6.SJL and OT-I transgenic mice were crossed to obtain OTISJL mice, while CD11c-Cre animals were crossed with floxed TGF-β receptor II (Tgfbr2^{fl/fl}) mice. The generation of prosaposin-deficient mice (pSAP-KO) has been reported previously. All animals were maintained in the animal barrier facility of Harvard Medical School (HMS), and all animal procedures were approved by the IACUC at HMS (approval number IS00001618).

### Cells

OVA-expressing MCA101 fibrosarcoma cells were developed by Dr. Clotilde Théry (Institut Curie, Paris, France) and have been described previously. In brief, MCA101/OVAC1C2 expresses the model antigen ovalbumin coupled to the C1C2 domain of milk fat globule EGF factor VIII (MFG-E8)/lactadherin, which targets ovalbumin to PS-expressing vesicles. B16F10 melanoma cells were procured from American Type Culture Collection (USA). Tumor cells were cultured in Dulbecco's Modified Eagle's Medium (DMEM) supplemented with 10% FBS, β-mercaptoethanol (55 mM; Gibco), penicillin (100 U/ml) and streptomycin (100 μg/ml), sodium pyruvate (1 mM), HEPES (100 mM), and hygromycin (300 μg/ml; Gibco). DC2.4 cells were purchased from Millipore Sigma and were cultured in complete RPMI medium containing 10% FBS, penicillin (100 U/ml) and streptomycin (100 μg/ml), sodium pyruvate (1 mM), L-glutamine (2 mM), β-mercaptoethanol (50 μM), and HEPES (100 mM). All cells were cultured in an incubator at 37° C., 5% CO₂, and 95% relative humidity.

### Antibodies

Antibodies directed against mouse CD8 (53-6.7), CD3 (145-2C11), and Fc block reagent (anti-mouse CD16/32) were purchased from BD Biosciences (USA). Antibodies against murine CD45.1 (A20), CD103 (2E7), LAMP-1 (1D4B), CD11b (M1/70), F4/80 (QA17A29), CD24 (QA20A91), IFNγ (XMG1.2), CD11c (N418), IA/IE (M5/114.15.2), XCR1 (ZET), SIRP1α(P84), K^{b}-SIINFEKL (25-D1.16) were purchased from BioLegend (USA). Antibodies against human CD146 (P1H12), CD8 (SK1), CD11c (Bul5), CD11b (QA20A58), and LAMP-1 (H4A3) were from BioLegend (USA). Anti-mouse DEC205 (NLDC-145) and isotype control (BE0094) antibody were purchased from BioXCell (USA). Anti-mouse and human pSAP antibody (polyclonal) were purchased from Proteintech (Japan), and anti-mouse/human sortilin (polyclonal) was purchased from Abcam (USA). Fluorochrome-labeled HLA-A2 tetramers loaded with peptides from MART (AA 26-35; ELAGIGILTV, SEQ ID NO: 21), NY-ESO-1 (AA 157-165; SLLMWITQV (SEQ ID NO: 22)), tyrosinase (AA 369-377; YMDGTMSQV (SEQ ID NO: 23)), and gp100 (AA 209-217; ITDQVPFSV (SEQ ID NO: 24)) were procured from Tetramer Shop (Denmark).

### Transmission Electron Microscopy (TEM)

Apoptotic vesicles were generated from the methylcholanthrene (MCA)-induced murine fibrosarcoma cell line MCA101. Cells were washed thoroughly, resuspended in complete RPMI 1640 medium, and subjected to irradiation using a cesium source (100 Gy). Successful apoptosis induction was confirmed by flow cytometry using annexin V staining. Subsequently, vesicles were purified from cell culture supernatants using differential ultracentrifugation to generate 100,000 g pellets as previously described. For electron microscopy, apoptotic bodies were fixed in 4% paraformaldehyde and 2.5% glutaraldehyde and embedded in Spurr's resin according to the manufacturer's protocol (Millipore Sigma). Sections were contrasted using lead citrate and analyzed using a Zeiss EM10 microscope.

### Calcein Leakage Assay

Apoptotic bodies were first resuspended in a buffer containing 50 mM calcein and subsequently passed through a liposome extruder using a membrane with 100 nm pores. Upon extrusion, the ˜400 nm vesicles were forced to transiently open their lipid bilayers to change their size according to the 100 nm pores used. During this process, extruded vesicles incorporated the calcein fluorochrome. To test saposin-induced vesicle disintegration, calcein-loaded apoptotic bodies were incubated with the four different saposins (5 ug/ml) in 50 mM sodium acetate buffer at a pH of 4.5 to facilitate saposin activity. Saposin-induced calcein release corresponded with increased fluorescence, which was measured using a spectrofluorometer. As a negative control, apoptotic bodies were incubated with bovine serum albumin (BSA). Percentage of calcein leakage was calculated using the fluorescence signal as a proportion of maximum calcein release induced by Triton X-100 and the minimum baseline levels.

### Generation of Murine Bone Marrow-Derived Dendritic Cells

Mouse tibiae and femora were isolated and flushed with ice-cold RPMI 1640 containing 5% FBS, 100 mM HEPES, and cells were then filtered through a 70 μm cell strainer. Red blood cells were lysed using red blood cell lysis buffer (155 mM NH₄Cl, 10 mM KHCO₃, 0.1 mM EDTA), and bone marrow cells were then plated at a density of 6-8×10⁶ cells per 10 cm petri dish in complete RPMI 1640 medium (10% FBS, 100 U/ml penicillin and 100 ug/ml streptomycin, 2 mM L-Glutamine, 1 mM sodium pyruvate, and 0.0012% 2-mercaptoethanol) in the presence of GM-CSF (20 ng/ml, PeproTech). Cells were cultured at 37° C., 5% CO₂, and 95% relative humidity for 7 days. Thereafter, floating BM-derived dendritic cells (BMDCs) were harvested by gently flushing the cells from the plate, and cell viability was assessed using trypan blue staining.

### Confocal Microscopy

For confocal imaging, MCA101 cells were labeled with 5 M CFSE and were γ-irradiated to generate apoptotic bodies as described above. 1×10⁵ BMDCs were cocultured with 5×10⁵ CFSE-labeled apoptotic MCA101 cells for different incubation times in poly-L-lysine-treated 8-chamber slides (Millipore Sigma) at 37° C., 5% CO₂, and 95% relative humidity. BMDCs were then permeabilized with 0.2% Triton X-100, blocked with PBS containing 2% FBS and 10% goat serum. Cells were further stained overnight with rabbit anti-LAMP1 antibody (Abcam, cat. no.: Ab24170) followed by staining with anti-rabbit-Alexa555 antibody (Molecular Probes, cat. no.: A21429). All samples were mounted with ProLong® Gold antifade mounting medium including DAPI (Life Technologies). Images were taken using an Olympus FV1000 confocal microscope. Images were analyzed using ImageJ/Fiji software v2.0 (NIH). For z-axis image reconstruction, 10 confocal sections, 0.5 m apart, were assembled using ImageJ/Fiji software.

### In Vitro CD8 T Cell Proliferation Assay

Preparation of BMDCs and apoptotic MCA101 cells was performed as described above. Naive CD8 T cells were purified from ovalbumin-specific TCR-transgenic OT-ISJL mice using a CD8 T cell isolation kit (Miltenyi Biotec). OT-I T cells were suspended in PBS (10⁷ cells/ml) and were incubated with CFSE (Molecular Probes; 5 mM) for 5 min at room temperature, and an equal volume of FBS was added to quench excess CFSE. To test CD8 T cell proliferation, BMDCs were pulsed with apoptotic MCA101 cells at a ratio of 1:5 for 4 hours, and thereafter, 5×10⁴ BMDCs were cocultured with 1×10⁵ CFSE-labeled OT-I CD8 T cells. In a separate experimental setting, pSAP-deficient BMDCs were pulsed with 10 μg/ml recombinant prosaposin. Cells were cultured in 96-well U-bottom plates at 37° C., 5% CO₂, and 95% relative humidity for 4 days. The proliferation of CFSE-labeled OT-I CD8 T cells was measured using flow cytometry, while soluble ovalbumin (50 μg/ml) was used as a positive control.

### Tumor Cell Inoculation

Mice were subcutaneously injected with 1×10⁶ live MCA101 tumor cells in the shaved right flank. In separate experiments, mice were immunized with 4×10⁵ irradiated MCA101 tumor cells in the left flank 7 days before live tumor challenge. For both experimental settings, tumor growth was monitored with calipers every 2 days, and a tumor volume of 2,000 mm³ was considered as an endpoint.

### Flow Cytometry and Cell Purification

Subcutaneous tumors were cut into small pieces and were digested in 20 ml of RPMI containing collagenase D (400 μg/ml), collagenase VIII (400 μg/ml), and 2% FCS for 1 hour at 37° C. Subsequently, 70% and 30% percoll density gradients were used to remove debris and dead cells, and single cell suspensions were collected from the gradient interface. Spleens and tumor-draining lymph nodes (dLNs) were cut and dissociated with 10 U/ml collagenase I and 30 U/mL DNase I (Millipore Sigma) for 45 min at 37° C., and single cell suspensions were collected by passing cells through 70-μm cell strainers. Red blood cells from all samples were removed using erythrocyte lysis buffer.

For flow cytometry experiments, single cell suspensions from tumor, spleen, and dLN were blocked with mouse Fc block reagent (BD Biosciences) for 15 min at 4° C., and were then stained with antibodies directed against CD45, CD8, LAMP-1, CD11c, CD24, CD103, CD11b, MHC-II, H-2K^{b}-SIINFEKL, F4/80, XCR-1, and SIRP1α. For IFN-γ staining, cells were stimulated with PMA (50 ng/ml) and Ionomycin (500 ng/ml; Sigma-Aldrich) for 4 hours in the presence of GolgiStop (BD Biosciences). Next, cells were surface stained, fixed, and incubated with anti-IFN-γ antibody using the Cytofix/Cytoperm kit (BD Biosciences) following the manufacturer's protocol. For tetramer staining, 2×10⁶ cells suspended in 50 μL PBS were incubated with antigen-specific MHC-I OVA tetramer (SIINFEKL; 1:50 dilution) for 15 min at 37° C. in the dark. Single cell suspensions from human melanoma samples were surface stained with antibodies against CD146, CD8, CD11b, CD11c, CD45, and LAMP-1, while intracellular staining for IFN-γ was performed using the Cytofix/Cytoperm kit (BD) following the manufacturer's protocol. In a separate set of experiments, human melanoma samples were stained with fluorochrome-labeled HLA-A2 tetramers loaded with epitopes from MART (AA 26-35; ELAGIGILTV), NY-ESO-1 (AA 157-165; SLLMWITQV), tyrosinase (AA 369-377; YMDGTMSQV), and gp100 (AA 209-217; ITDQVPFSV), incubating 1×10⁵ cells with the respective tetramers for 45 min at 37° C. in the dark. Data were acquired using a BD FACS Canto II (BD Biosciences) and analyzed with FlowJo software (Treestar).

Cell purifications were performed either by FACS-assisted cell sorting or by magnetic-activated cell sorting (MACS). For magnetic cell separation, cells were suspended in MACS buffer, and DCs and naive CD8 T cells were isolated using the respective beads following the manufacturer's instructions (Miltenyi Biotec). Human CD146⁺ melanoma cells, CD8⁺ T cells, and CD11c/b⁺ myeloid cells, as well as mouse cDC1 and cDC2 from tumor and spleen were FACS-sorted using a BD FACS Aria II, and purity of cells was determined by post-sort analysis based on flow cytometry.

### Antigen Uptake and Processing Assays

To test their phagocytosis and antigen processing capacities, DCs isolated from tumor, spleen, or dLNs were incubated with either FITC-dextran (1 mg/ml), or DQ-ovalbumin (0.5 mg/ml) for 1 hour in a CO₂ incubator at 37° C. DQ-OVA is a self-quenching reagent, which fluoresces only after proteolytic cleavage by lysosomal enzymes. While FITC-dextran uptake defines the extent of endocytosis, any fluorescence in the green channel derived from cleaved DQ-OVA represents the antigen processing abilities of DCs. Background signal was determined by culturing control DCs at 4° C., and mean fluorescence intensity (MFI) was subsequently measured by flow cytometry.

### In Vivo CD8 T Cell Priming

OT-I CD8 T cells were isolated from OT-ISJL mice using MACS sorting (Miltenyi Biotec) and labeled with 5 M CFSE in PBS. CFSE-labeled cells were tested for viability using trypan blue staining, and 5×10⁶ viable OT-I CD8 T cells were then intravenously administered to WT or pSAP-KO mice. On the following day, 5×10⁶ irradiated MCA101-OVA cells were subcutaneously injected into each recipient's left flank, and draining inguinal lymph nodes (dLN) were harvested four days later. Proliferation of OT-I CD8 T cells and the production of IFN-γ were measured using flow cytometry.

### Bone Marrow Chimera Generation

Bone marrow (BM) cells were prepared from tibias and femurs of WT, pSAP-KO, CD11c-Cre ×Tgfbr2^{flox/flox}, and littermate control mice as described above. WT mice were irradiated using a cesium source (900 rad), and irradiated mice were injected with 1-3×10⁶ BM cells from pSAP-KO, CD11c-Cre×Tgfbr2^{flox/flox}, or WT littermate control mice. Chimera were treated with 2 mg/ml neomycin (Sigma-Aldrich) in drinking water for 4 weeks. Eight to twelve weeks after BM transfer, mice were assessed for chimerism and subsequently used for tumor experiments.

### Immunoprecipitation and Western Blot

FACS-sorted DCs were lysed in 50 mM Tris (pH 8.0), containing 150 mM NaCl, 1% Triton X-100, 0.1% deoxycholic acid and 1× protease inhibitors (Roche). Anti-pSAP antibody was coupled with amine-reactive resin following the manufacturer's instructions (Thermo Fisher Scientific). Subsequently, cell lysates were incubated overnight with resin-conjugated anti-pSAP antibody, and bound protein complexes were enriched using column purification (Thermo Fisher Scientific) following the manufacturer's protocol. For immunoblot analysis, cells were lysed in RIPA buffer containing 1× protease and phosphatase inhibitors, and protein concentrations were determined using a BCA protein quantification kit (Bio-Rad). Samples were diluted in Laemmli's sample buffer (Bio-Rad) and were loaded onto 15% Mini-PROTEAN TGX precast gels (Bio-Rad). Electrophoresis was carried out at 120 V for 1 hour in tris-glycine buffer. Separated proteins were transferred to nitrocellulose membranes, and the membranes were blocked in 5% BSA dissolved in TBST buffer (tris-buffered saline, containing 20 mM Tris-HCl, pH 7.6, 150 mM NaCl, 0.1% Tween 20). After blocking, membranes were incubated with antibodies against either mouse pSAP, sortilin, β-actin, or human pSAP at 4° C. overnight. Thereafter, membranes were washed three times with TBST and were incubated with 1:5,000 diluted HRP-labeled anti-rabbit antibody (Thermo Fisher Scientific) for 1 hour. After additional three washes, membranes were exposed to chemiluminescent substrate and imaged upon exposure to X-ray films in the dark.

### Endoglycosidase H (Endo H) Digestion Assay

FACS-sorted DCs were lysed in RIPA buffer, and cell lysates were subjected to Endo H digestion according to the manufacturer's protocol (New England Biolabs). Briefly, 100 g of protein lysate was denatured using the glycoprotein-denaturing buffer supplied by the manufacturer for 10 minutes at 99° C. Subsequently, samples were cooled at 4° C., and 500 U of Endo H was added to each sample. Glycan digestion was carried out at 37° C. for at least 12 hours, and the release of glycans from pSAP was determined by immunoblot analysis.

### ELISA

pSAP concentrations were determined using sandwich ELISA following the manufacturer's instructions (Abcam). Briefly, plates precoated with antibody specific for pSAP were incubated with standards and samples for 2 hours at room temperature. The wells were washed three times, and biotinylated anti-mouse pSAP antibody was added to each well (1:500 dilution). Thereafter, plates were washed three times, and HRP-conjugated streptavidin was added to each well, prior to incubation for 1 hour at room temperature. TMB substrate solution was added to each well, and the color reaction was stopped by using 2N H₂SO₄. Plates were then measured for optical density at 450 nm using a spectrophotometer.

### Proximity Ligation Assay (PLA)

FACS-sorted DC subsets from tumor and spleen were plated on eight-chambered slides for 45 min at 37° C., and adherent cells were washed and fixed for 15 min with 2% paraformaldehyde. Cells were permeabilized with 0.5% saponin in PBS for 30 min. PLA was carried out with Duolink® in situ detection reagents following the manufacturer's instructions (Millipore Sigma). Anti-pSAP antibody was labeled with plus probes, while anti-sortilin antibodies were labelled with minus probes. For PLA reaction, cells were blocked with Duolink® blocking solution for 1 hour at room temperature and subsequently incubated with the respective PLA probes for 2 hours. After washing cells three times, all probe-labeled cells were subjected to PLA signal amplification. Finally, cells were mounted on slides with DAPI-containing mounting reagent. Images were taken using an Olympus Fluoview (FV) 1000 confocal microscope, and data were analyzed using ImageJ software (NIH).

qRT-PCR for Glycosyltransferase and Glycosidase Genes

CD11c⁺ DCs from spleen and tumor were purified using FACS sorting. DC2.4 cells were collected upon trypsinization after 4 days of 2.5 ng/ml TGF-β stimulation. Expression of genes involved in glycosylation were quantified by quantitative real-time PCR (qRT-PCR) using the glycosylation RT² Profiler PCR Array according to the manufacturer's instructions (PAMM-046Z, Qiagen). Briefly, RNA was isolated from DCs using the RNeasy Kit (Qiagen), and 50 ng RNA was converted to cDNA using the RT² First Strand Kit (Qiagen). The cDNAs were subjected to amplification using gene-specific primers and SYBR Green (RT² SYBR Green Master Mix, Qiagen) in an Ab7100 Real-Time PCR System (Thermo Fisher Scientific). Amplification of the gene encoding for β-actin served as internal control, and relative gene expression levels were calculated using the 2^{-ΔΔCT} method.

### Mass Spectrometry

FACS-sorted CD11c⁺ DCs from spleen and tumor were lysed, and pSAP was immunoprecipitated as described above, prior to loading onto 12% acrylamide gels and visualization using coomassie blue staining. pSAP bands corresponding to a molecular weight of 65 kDa and 75 kDa were excised and incubated at room temperature for 5 min with 200 μl of a freshly prepared 50 mM (3.91 mg/ml) ammonium bicarbonate solution and 200 μl of acetonitrile (100%). Subsequently, samples were dried using a SpcedVac and incubated with freshly prepared 10 mM (1.54 mg/ml) 1,4-dithiothreitol (DTT, Sigma) at 50° C. for 30 min. Thereafter, pSAP was alkylated with 55 mM (10.2 mg/ml) iodoacetamide (IAA, Sigma, #16125) at room temperature for 30 mins, and peptides were subsequently cleaved by incubating samples with a 20 μg/ml solution of TPCK-treated trypsin (Sigma, #4352157) at 37° C. overnight. Trypsin digestion was stopped by the addition of ˜2 drops of 5% acetic acid, and samples were added to a C18 Sep-Pak (200 rng) column (Waters) preconditioned with a solution of methanol, 1-propanol, and 5% acetic acid (2:2:1). Reaction tubes were washed with 1 mL of 5% acetic acid and added to the column, followed by an additional 5 mL wash of 5% acetic acid. Each column was placed in a 15 mL glass tube, and glycopeptides were eluted using 2 mL of 20% 1-propanol, 2 mL of 40% 1-propanol, and 2 mL of 100% 1-propanol. The eluted fractions were pooled and placed in a SpeedVac to remove the excess organic solvent, followed by lyophilization. For N-glycan release, dried peptides were treated with 1 μl of PNGase F in 200 μl of 50 mM ammonium bicarbonate solution overnight at 37° C. Permethylated glycans were resuspended in 25 μL of 75% methanol and spotted in a 1:1 ratio with DHB matrix on an MTP 384 polished steel target plate (Bruker Daltonics) as previously described. MALDI-TOF MS data were acquired based on a Bruker Ultraflex H instrument using Flex Control software in the reflective positive mode. For N-glycans, a mass/charge (m/z) range of 1,000-5,000 kDa was collected, and twenty independent captures were obtained from each sample and averaged to create the final combined spectra file. Data were exported in .msd fornat using Flex Analysis software for subsequent annotation.

### TGF-β In Vitro Stimulation

DC2.4 cells were seeded in complete RPMI at 75% confluency in 24-well plates. The following day, cells were washed and cultured in complete RPMI containing 0, 2.5, or 10 ng/ml recombinant TGF-β for 4 days. Culture supernatant was concentrated using 10 kDa protein concentrators, and the amount of prosaposin released was quantified using ELISA.

### Melanoma Patient Samples

Dissociated whole tumor samples were purchased from Discovery Life Sciences (USA). Melanoma, myeloid, and CD8 T cells were FACS-sorted as described above. Melanoma cells were γ-irradiated (10,000 rad), and induction of apoptosis was monitored by Annexin V staining using flow cytometry. 30,000 DCs were cocultured with 150,000 CD8 T cells (1:5 ratio), and 1×10⁵ irradiated melanoma cells were used as antigen source. To test the function of pSAP, cultures were stimulated with 5 g recombinant human pSAP (Abcam). Cells were cocultured for 5 days, and CD8 T cell activation was quantified by cytokine and tetramer staining using flow cytometry as described above.

### Generation of Monocyte-Derived DCs

Peripheral blood mononuclear cell samples were procured from Discovery Life Sciences (USA). Monocytes were isolated from mononuclear cell fractions using CD14 magnetic microbeads, according to the manufacturer's instructions (Miltenyi Biotec). Isolated monocytes were cultured in tissue-culture dishes at 0.4×10⁶ cells/mL in complete RPMI 1640 medium, containing recombinant human IL-4 (50 ng/mL; R&D Systems) and recombinant human GM-CSF (100 ng/mL; R&D Systems), with 1 supplement of fresh medium and cytokines on day 3 of culture. Cells were harvested on day 6 to be subsequently used for immunoblotting and PLA.

Chemical Coupling of pSAP with Anti-DEC205 Antibody

Recombinant pSAP was coupled with anti-DEC205 antibody activated with sulfo-SMCC (sulfosuccinimidyl 4-(N-maleimidomethyl) cyclohexane-1-carboxylate; Pierce Chemical Co.), following the manufacturer's protocol. pSAP was reduced with TCEP (Tris[2-carboxyethyl]phosphine hydrochloride) immobilized onto 4% crosslinked beaded agarose for 90 min at room temperature. In parallel, anti-DEC205 or isotype control antibodies were maleimide-activated by incubation with sulfo-SMCC for 30 min at 37° C. Excess of salts were removed by desalting columns, and subsequently, reduced pSAP and maleimide-activated antibodies were incubated overnight at 4° C. Antibody/pSAP conjugates were purified by passing through 150 kD protein concentrators (Thermo Fisher Scientific), and the purity and concentrations of antibody/pSAP conjugates were determined using SDS-PAGE and ELISA. To test binding of anti-DEC205/pSAP to the DEC205 receptor, pSAP-KO BMDCs were incubated with anti-DEC205/pSAP conjugates, prior to staining with anti-pSAP antibody. Isotype antibody-coupled pSAP was used as negative control, while direct staining of DCs with anti-DEC205 antibody was used as positive control. To examine CD8 T cell activation, pSAP-KO BMDCs were pulsed with irradiated MCA101-OVA cells and cocultured with OT-I CD8 T cells in the presence of either anti-DEC205/pSAP or isotype/pSAP conjugates. To investigate the in vivo delivery of anti-DEC205/pSAP to tumor DCs, MCA101 tumor-bearing pSAP-KO BM chimeric mice were injected with 100 μg pSAP conjugated to either anti-DEC205 or isotype antibodies on days 9 and 13 after tumor cell inoculation. pSAP delivery to cDC1 and cDC2 was quantified using flow cytometry. In another set of experiments, B16F10-harboring mice were injected with 100 g anti-DEC205/pSAP conjugate combined with 100 g of either anti-PD-L1 or isotype control antibody for six injections every three days.

### Statistical Analyses

All data are presented as mean and standard deviation of the mean (SD). The statistical significance of differences was calculated using a two-tailed Student's t-test for both paired and unpaired samples using Prism 9.0 (GraphPad software). p-values≤0.05 were considered significant (*p<0.05; **p<0.01; ***p<0.001; ****p<0.0001).

### Example 3

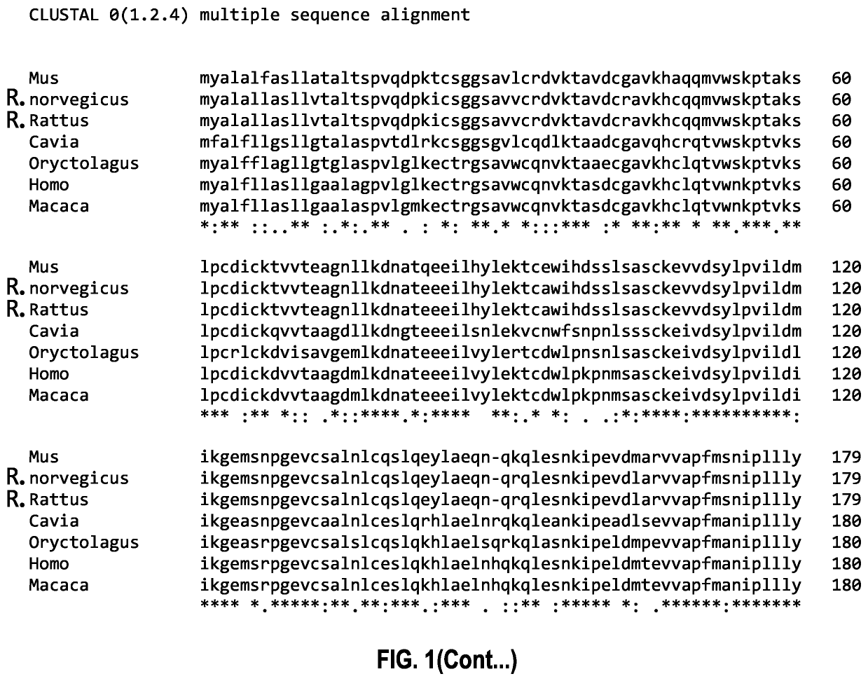

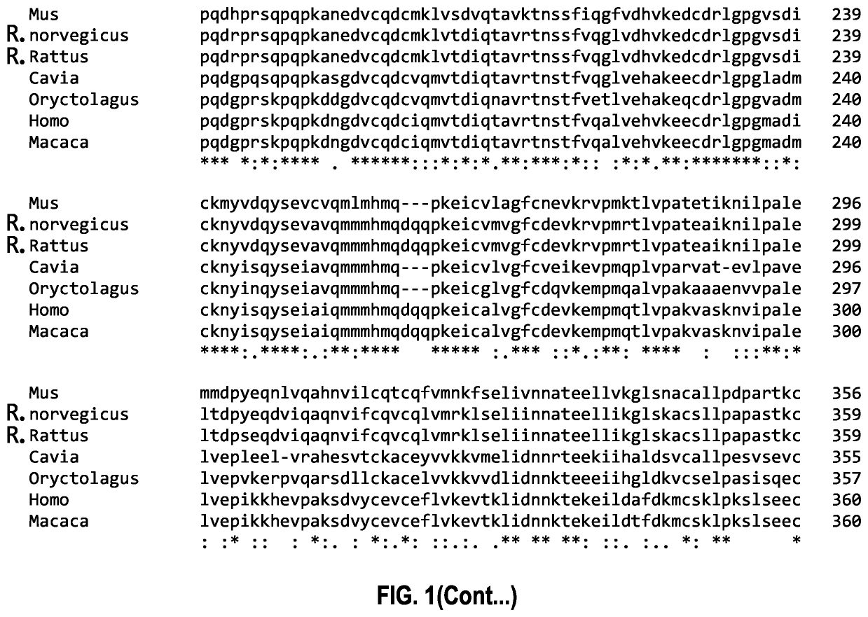

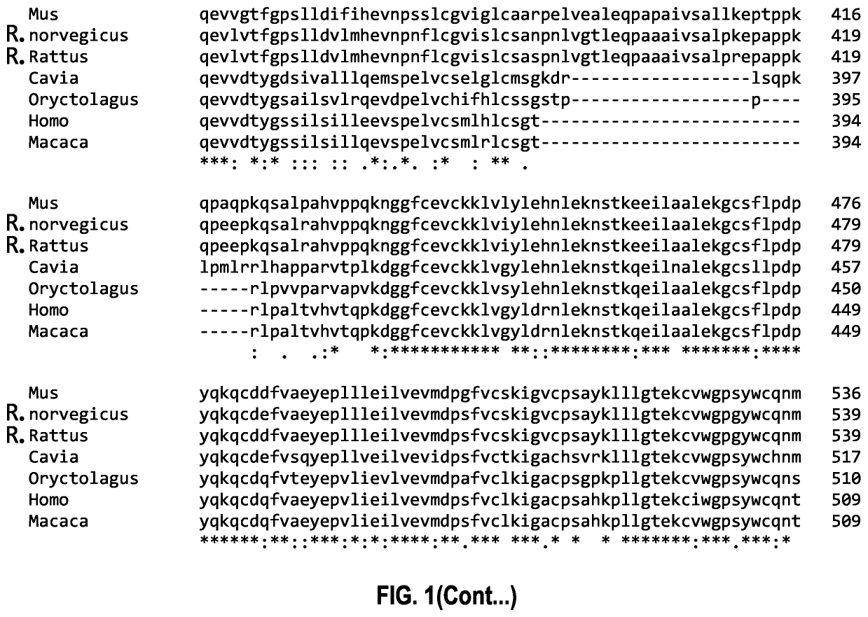

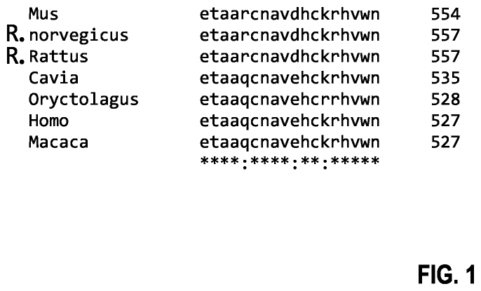

Sequences from Alignment in FIG. 1:

Prosaposin (PSAP) Homo sapiens (protein: NP_001035930.1, NCBI
Gene ID: 5660), SEQ ID NO: 5:
myalfllasl lgaalagpvl glkectrgsa vwcqnvktas dcgavkhclq tvwnkptvks
lpcdickdvv taagdmlkdn ateeeilvyl ektcdwlpkp nmsasckeiv dsylpvildi
ikgemsrpge vcsalnlces lqkhlaelnh qkqlesnkip eldmtevvap fmanipllly
pqdgprskpq pkdngdvcqd ciqmvtdiqt avrtnstfvq alvehvkeec drlgpgmadi
cknyisqyse iaiqmmmhmq dqqpkeical vgfcdevkem pmqtlvpakv asknvipale
lvepikkhev paksdvycev ceflvkevtk lidnnkteke ildafdkmcs klpkslseec
qevvdtygss ilsilleevs pelvcsmlhl csgtrlpalt vhvtqpkdgg fcevckklvg
yldrnlekns tkqeilaale kgcsflpdpy qkqcdqfvae yepvlieilv evmdpsfvcl
kigacpsahk pllgtekciw gpsywcqnte taaqcnaveh ckrhvwn
Prosaposin Mus musculus (protein: NP_001139592.1, NCBI Gene ID:
19156), SEQ ID NO: 6:
myalalfasl lataltspvq dpktcsggsa vlcrdvktav dcgavkhcqq mvwskptaks
lpcdicktvv teagnllkdn atqeeilhyl ektcewihds slsasckevv dsylpvildm
ikgemsnpge vcsalnlcqs lqeylaeqnq kqlesnkipe vdmarvvapf msniplllyp
qdhprsqpqp kanedvcqdc mklvsdvqta vktnssfiqg fvdhvkedcd rlgpgvsdic
knyvdqysev cvqmlmhmqp keicvlagfc nevkrvpmkt lvpatetikn ilpalemmdp
yeqnlvqahn vilcqtcqfv mnkfselivn nateellvkg lsnacallpd partkcqevv
gtfgpslldi fihevnpssl cgviglcaar pelvealeqp apaivsallk eptppkqpaq
pkqsalpahv ppqknggfce vckklvlyle hnleknstke eilaalekgc sflpdpyqkq
cddfvaeyep llleilvevm dpgfvcskig vcpsayklll gtekcvwgps ywcqnmetaa
rcnavdhckr hvwn
Prosaposin Rattus norvegicus (protein NP_001177165.1, NCBI Gene
ID: 25524), SEQ ID NO: 7:
myalallasl lvtaltspvq dpkicsggsa vvcrdvktav dcravkhcqq mvwskptaks
lpcdicktvv teagnllkdn ateeeilhyl ektcawihds slsasckevv dsylpvildm
ikgemsnpge vcsalnlcqs lqeylaeqnq rqlesnkipe vdlarvvapf msniplllyp
qdrprsqpqp kanedvcqdc mklvtdiqta vrtnssfvqg lvdhvkedcd rlgpgvsdic
knyvdqysev avqmmmhmqd qqpkeicvmv gfcdevkrvp mrtlvpatea iknilpalel
tdpyeqdviq aqnvifcqvc qlvmrklsel iinnateell ikglskacsl lpapastkcq
evlvtfgpsl ldvlmhevnp nflcgvislc sanpnlvgtl eqpaaaivsa lpkepappkq
peepkqsalr ahvppqkngg fcevckklvi ylehnlekns tkeeilaale kgcsflpdpy
qkqcdefvae yepllleilv evmdpsfvcs kigvcpsayk lllgtekcvw gpgywcqnme
taarcnavdh ckrhvwn
Prosaposin Rattus rattus (protein XP_032744181.1, NCBI Gene ID:
116886842), SEQ ID NO: 8:
myalallasl lvtaltspvq dpkicsggsa vvcrdvktav dcravkhcqq mvwskptaks
lpcdicktvv teagnllkdn ateeeilhyl ektcawihds slsasckevv dsylpvildm
ikgemsnpge vcsalnlcqs lqeylaeqnq rqlesnkipe vdlarvvapf msniplllyp
qdrprsqpqp kanedvcqdc mklvtdiqta vrtnssfvqg lvdhvkedcd rlgpgvsdic
knyvdqysev avqmmmhmqd qqpkeicvmv gfcdevkrvp mrtlvpatea iknilpalel
tdpseqdviq aqnvifcqvc qlvmrklsel iinnateell ikglskacsl lpapastkcq
evlvtfgpsl ldvlmhevnp nflcgvislc saspnlvgtl eqpaaaivsa lprepappkq
peepkqsalr ahvppqkngg fcevckklvi ylehnlekns tkeeilaale kgcsflpdpy
qkqcddfvae yepllleilv evmdpsfvcs kigvcpsayk lllgtekcvw gpgywcqnme
taarcnavdh ckrhvwn
Prosaposin Cavia porcellus (protein: XP_005005379.1, NCBI Gene
ID: 100713024), SEQ ID NO: 9:
mfalfllgsl lgtalaspvt dlrkcsggsg vlcqdlktaa dcgavqhcrq tvwskptvks
lpcdickqvv taagdllkdn gteeeilsnl ekvonwfsnp nlsssckeiv dsylpvildm
ikgeasnpge vcaalnlces lqrhlaelnr qkqleankip eadlsevvap fmanipllly
pqdgpqsqpq pkasgdvcqd cvqmvtdiqt avrtnstfvq glvehakeec drlgpgladm
cknyisqyse iavqmmmhmq pkeicvlvgf cveikevpmq plvparvate vlpavelvep
leelvrahes vtckaceyvv kkvmelidnn rteekiihal dsvcallpes vsevcqevvd
tygdsivall lqemspelvc selglcmsgk drlsqpklpm lrrlhappar vtplkdggfc
evckklvgyl ehnleknstk qeilnalekg csllpdpyqk qcdefvsqye pllveilvev
idpsfvctki gachsvrkll lgtekcvwgp sywchnmeta aqcnavehck rhvwn
Prosaposin Oryctolagus cuniculus (protein: XP_008268218.1, NCBI
Gene ID: 100359322), SEQ ID NO: 10:
myalfflagl lgtglaspvl glkectrgsa vwcqnvktaa ecgavkhclq tvwskptvks
lpcrlckdvi savgemlkdn ateeeilvyl ertcdwlpns nlsasckeiv dsylpvildl
ikgeasrpge vcsalslcqs lqkhlaelsq rkqlasnkip eldmpevvap fmanipllly
pqdgprskpq pkddgdvcqd cvqmvtdiqn avrtnstive tlvehakeqc drlgpgvadm
cknyinqyse iavqmmmhmq pkeicglvgf cdqvkempmq alvpakaaae nvvpalelve
pvkerpvqar sdllckacel vvkkvvdlid nnkteeeiih gldkvcselp asisqecqev
vdtygsails vlrqevdpel vchifhlcss gstpprlpvv parvapvkdg gfcevckklv
sylehnlekn stkqeilaal ekgcsflpdp yqkqcdqfvt eyepvlievl vevmdpafvc
lkigacpsgp kpllgtekcv wgpsywcqns etaaqcnave hcrrhvwn
Prosaposin Macaca mulatta (protein: XP_015002912.1, NCBI Gene
ID: 709510), SEQ ID NO: 11:
myalfllasl lgaalaspvl gmkectrgsa vwcqnvktas dcgavkhclq tvwnkptvks
lpcdickdvv taagdmlkdn ateeeilvyl ektcdwlpkp nmsasckeiv dsylpvildi
ikgemsrpge vcsalnlces lqkhlaelnh qkqlesnkip eldmtevvap fmanipllly
pqdgprskpq pkdngdvcqd ciqmvtdiqt avrtnstfvq alvehvkeec drlgpgmadm
cknyisqyse iaiqmmmhmq dqqpkeical vgfcdevkem pmqtlvpakv asknvipale
lvepikkhev paksdvycev ceflvkevtk lidnnkteke ildtfdkmcs klpkslseec
qevvdtygss ilsillqevs pelvcsmlrl csgtrlpalt vhvtqpkdgg fcevckklvg
yldrnlekns tkqeilaale kgcsflpdpy qkqcdqfvae yepvlieilv evmdpsfvcl
kigacpsahk pllgtekcvw gpsywcqnte taaqcnaveh ckrhvwn
Sap-A domain consensus sequence (smart00162: SAPA, part of the
SapA superfamily c102494), SEQ ID NO: 12:
GPKRCTWGPSVWCQNLETASQCNAVKHCLQRVWS
Sap-A domain consensus sequence (pfam02199: SAPA, part of the
SapA superfamily c102494), SEQ ID NO: 13:
TKECTWGPSYWCQSLETAKECGAVEHCQQHVWN
Sap-B domains consensus sequence (smart00741, member of the
superfamily cl33398), SEQ ID NO: 14:
LLCELCEFVVKQLENLLKDNKT-EEEIKKALEKVCKKLP-KS-
LSDQCKEFVDQYGPEIIDLLEQGLD--PKDVCQKLGL

What is claimed herein is:

1. A composition comprising: a saposin or prosaposin targeted for delivery to a dendritic cell.

2. The composition of claim 1, wherein the saposin or prosaposin comprises an antigen binding moiety that binds a dendritic cell antigen.

3. The composition of claim 2, wherein the saposin or prosaposin is chemically conjugated to the antigen binding moiety, optionally via a chemical or peptide linker.

4. The composition of claim 2, wherein the dendritic cell antigen comprises DEC205.

5. The composition of claim 2, wherein the antigen binding moiety comprises an antibody or antigen binding fragment thereof.

6. The composition of claim 1, wherein the saposin comprises saposin A, saposin B, saposin C, and/or saposin D.

7. The composition of claim 1, further comprising a pharmaceutically acceptable carrier.

8. A fusion protein comprising a saposin or prosaposin conjugated to a moiety that targets the fusion protein to dendritic cells.

9. A method of treating cancer comprising administering the composition of any one of claims 1-8 to a subject in need thereof.

10. A method of enhancing anti-tumor immunity in a subject, the method comprising administering a composition of any one of claims 1-9 to a subject having cancer, wherein the anti-tumor immunity is increased by at least 10% as compared to the anti-tumor immunity in a subject not administered the composition of any one of claims 1-9.

11. The method of claim 10, wherein the anti-tumor immunity is assessed by measuring T lymphocyte stimulation.

12. The method of claim 10, wherein the tumor volume is decreased in the subject by at least 20%.

13. The method of claim 10, wherein the cancer is melanoma.

14. The method of claim 10, wherein antigen presentation by dendritic cells is increased.

15. The method of claim 10, wherein the saposin or prosaposin degrades apoptotic vesicles from the tumor.
